(19) United States
(12) Patent Application Publication
(10) Pub. No.: US 2026/0117258 A1
(43) Pub. Date: Apr. 30, 2026

(54) Novel Transposase System

(71) Applicant: Boehringer Ingelheim International GmbH, Ingelheim am Rhein (DE)

(72) Inventors: Moritz SCHMIDT, Erbach (DE); Daniel HEINZELMANN, Ulm (DE); Franziska REUSS, Ringschnait (DE); Simon FISCHER, Staig (DE); Patrick SCHULZ, Warthausen (DE)

(21) Appl. No.: 19/116,815

(22) PCT Filed: Sep. 29, 2023
(86) PCT No.: PCT/EP2023/077173

§ 371 Date: Mar. 28, 2025

(30) Foreign Application Priority Data
Sep. 30, 2022 (EP) 22199126.8
Jun. 7, 2023 (EP) 23178118.8

(51) Int. Cl. C12N 15/90; C12N 7/00; C12N 9/12; C12N 15/85; C12P 21/00

(52) U.S. Cl. CPC C12N 15/90; C12N 7/00; C12N 9/1241; C12N 15/85; C12P 21/00; C07K 2319/09; C12Y 207/07

(57) ABSTRACT

The invention relates to a novel functional transposase system. More specifically, the invention relates to a DNA transposon comprising a heterologous polynucleotide flanked by transposon flanking regions and to a recombinant transposase of Acyrthosiphon pisum (AP) or Aphis craccivora (AC) comprising an amino acid sequence of at least 90% sequence identity with SEQ ID NO: 4 fused to a heterologous nuclear localization signal, optionally comprising at least one activity improving mutation and to a polynucleotide or an expression vector encoding said DNA transposon or transposase as well as an expression system comprising the transposase/transposon pair. The invention also relates to methods for stably integrating a polynucleotide into cells or stably expressing a protein of interest using the transposase/transposon pair.

### SEQUENCE LISTING

The instant application contains a Sequence Listing which has been submitted electronically in XML format and is hereby incorporated by reference in its entirety. Said XML copy, created on Sep. 18, 2023, is named “118877P1140PC_sequence listing” and is 103.000 bytes in size.

### FIELD OF THE INVENTION

The invention relates to a novel functional transposase system. More specifically, the invention relates to a DNA transposon comprising a heterologous polynucleotide flanked by transposon flanking regions and to a recombinant transposase of Acyrthosiphon pisum (AP) or Aphis craccivora (AC) comprising an amino acid sequence of at least 90% sequence identity with SEQ ID NO: 4 fused to a heterologous nuclear localization signal, optionally comprising at least one activity improving mutation and to a polynucleotide or an expression vector encoding said DNA transposon or transposase as well as an expression system comprising the transposase/transposon pair. The invention also relates to methods for stably integrating a polynucleotide into cells or stably expressing a protein of interest using the transposase/transposon pair.

### BACKGROUND

The stable integration of transgenes into a host cell genome is of particular interest in the field of gene therapy, cell and genome engineering, cell line development and recombinant protein production. Integration of a transgene can for example be achieved by transfecting linearized DNA material into a cell, where integration events are based on randomly occurring genomic double strand breaks often resulting in the integration of DNA fragments or concatemers in an uncontrolled fashion at various genomic loci. Transposase enzymes, on the other hand, are proteins that recognize specific DNA sequences (inverted terminal repeat (ITR) sequences within a transposon flanking regions) and are able to cut and integrate the transposon cargo, along with the transposon flanking sequences comprising the ITRs reversibly into genomic loci at pre-defined sites harbouring a target sequence, preferably the short motif “TTAA”. Transposases usually target transcriptionally active and/or accessible genomic loci and result in a defined integration of the transposon cargo/transgene without fragmentation, concatemerisation, rearrangement or recombination. Thus transposase-mediated integration results in more stable recombinant cell lines with higher expression levels compared to random integration. By controlling the heterologous gene between the transposon flanking sequences, transposases can be modulated to integrate basically any DNA sequence into different genomic loci of a cell.

One of the first discovered functionally active Piggy-Bac transposase/transposon pair is derived from the looper moth Trichoplusia ni and was demonstrated to be active in a variety of cells and organisms. Another previously described transposase is a Tc1/mariner-type system, such as the hyperactive Sleeping Beauty SB100X derived from fish (Mates L et al., Nat Genet., 41(6):753-61 (2009)). Both relate to the class of DNA transposons or Class II transposons and use a “cut and paste” mechanism. By contrast, Class I transposons, also referred to as retrotransposons, use a “copy and paste” mechanism and include, e.g., non-LTR TRAS retrotransposons (Monti V. et al., Journal of Heredity, 104(4): 547-553 (2013)) and LTR retroelements (Bernet G. et al., Mobile Genetics Elements, 1(2): 97-102 (2011)). Most published genomic sequences harbour transposase/transposon-like sequences but only very few have since been demonstrated to be active. Mobile genetic elements, such as active transposase/transposon pairs, are a potential source of genomic instability and thus potentially detrimental to a cell or organism, resulting in the inactivation of most transposase/transposon elements over time e.g. via mutation, depletion, insertion or other genomic rearrangements. Only very few active transposase/transposon pairs have since been described. Active transposase/transposon have been discovered from Trichoplusia ni, Xenopus tropicalis, Bombyx mori and more recently from Oryzias latipes, Amyelois transitella, Heliothis virencens, Agrotis lpsilon and Helicoverpa armigera (US 2015/291975, US 2017/101629, US 2020/318121, US 2020/318135, US 2020/318107, Hikosaka, Mol Biol Evol 24, 2648-2656 (2007)). Those findings demonstrate that most of the experimentally tested transposase/transposon-like pairs from nature are no longer functional. For example, US 2020/031807 A1 reports that although PiggyBac-like transposases and transposons occur naturally in a wide range of organisms, transposition activity has been described for almost none of these. Like other transposable elements, PiggyBac transposase/transposon-like sequences are described by Bouallègue and Rouault et al., (Genome Biol. Evol., 2017, 9(2): 323-339) to be prone to indels, recombinations and mutations that inactivate many copies and further to show a large diversity. Yet, the authors focused on the genomic level and molecular evolution only without providing activity data. Tcl/mariner transposases are a further non-related class of transposases and mariner-like elements in genomic sequences are discussed by Bouallègue and Filée et al., (BMC Genomics, 2017, 18(1): 1-12) in silico and again without showing activity data. The existence of numerous transposase/transposon-like sequences does not provide any information about the presence of active transposase/transposon pairs and on the contrary adds to the complexity of identifying new active transposase/transposon pairs.

The increased availability of genomic sequences from variouss species and organisms opens the possibility to discover unknown transposase/transposon-like sequences. However, the identification of novel and functionally active transposase/transposon pairs is very challenging, because transposase/transposon sequences can be diverse with only little sequence homologies to existing sequences, making a sequence-based prediction on the activity and functionality of a transposase virtually impossible.

Also, very few expression systems using functional transposase/transposon pairs are commercially used, including a hyperactive piggy-Bac transposase (Transposagen/Lonza), a hyperactive piggy-Bac transposase fused to a chromatin reader element (DirectedLuck™, ProBiogen), the hyperactive transposases Leap-In® 1 and 2 (ATUM) or the hyperactive sleeping beauty transposase SB100X (Max-Delbruck Center; Mates L et al., Nat Genet., 41(6):753-61 (2009)). So, there is a need for further and functional transposase/transposon pairs especially for ones which are prone to further improvements, e.g. in view of stability and productivity of a cargo or transgenes encoded thereby.

### SUMMARY OF THE INVENTION

The present invention relates to novel transposase/transposon pairs, e.g. from Acyrthosiphon pisum (AP) or Aphis craccivora (AC). Each of the novel transposase/transposon pairs presents itself as an additional, highly efficient, orthogonal transposase/transposon pair, that is functionally active in eukaryotic cells and with a transposition efficacy that is at least comparable with the above mentioned commercially available transposase/transposon systems. The transposase according to the invention specifically recognizes the identified transposon ends comprising the inverted terminal repeats (ITRs), which are substantially different from any published transposase/transposon system, and transposition leads to the stable integration of any cargo polynucleotide (e.g., encoding a protein of interest, non-coding RNA, viral genome or gene(s)) into the host cell genome of interest.

In a first aspect the invention relates to a DNA transposon comprising a heterologous polynucleotide flanked by transposon flanking regions, wherein one transposon flanking region comprises the nucleotide sequence of SEQ ID NO: 38 at one transposon end; and the other transposon flanking region comprises the nucleotide sequence of SEQ ID NO: 39 at the other transposon end. The DNA transposon according to the invention is transposable by a transposase comprising the amino acid sequence of SEQ ID NO: 4.

In certain embodiments, the one transposon flanking region comprises the nucleotide sequence of SEQ ID NO: 40 at one transposon end; and the other transposon flanking region comprises the nucleotide sequence of SEQ ID NO: 41 at the other transposon end. Preferably, the one and the other transposon flanking region each further comprises at least one inverted internal repeat (IIR) comprising (i) an internal repeat motif comprising the sequence tggtctac and (ii) its reverse complementary sequence (iii) separated by six to three, preferably four nucleotides. More preferably the at least one inverted internal repeat (IIR) motif has the nucleotide sequence of SEQ ID NO: 43. In a preferred embodiment (a) the one transposon flanking region comprises the nucleotide sequence of SEQ ID NO: 45 or 46 and further an inverted internal repeat motif having the nucleotide sequence of SEQ ID NO: 43 separated by about 50 to 200 nucleotides; and/or the other transposon flanking region comprises the nucleotide sequence of SEQ ID NO: 47 or a sequence having at least 95% sequence identity with SEQ ID NO: 47, wherein the sequence comprises at least the nucleotide sequence of SEQ ID NO: 41 and the nucleotide sequence of SEQ ID NO: 43; or (b) the one and the other transposon flanking regions are inverted and comprise reverse complementary sequences of the sequences recited in (a).

In certain embodiments (a) the one transposon flanking region comprises a sequence having at least 85% nucleotide sequence identity with SEQ ID NO: 2, 8, 10 or 58 and/or the other transposon flanking region comprises a sequence having at least 90% nucleotide sequence identity with SEQ ID NO: 3, 9, 11 or 59; or (b) the one and the other transposon flanking regions are inverted and comprise reverse complementary sequences of the sequences recited in (a). Preferably the one transposon flanking region comprises the nucleotide sequence of SEQ ID NO: 2, 8, 10 or 58 or the nucleotide sequence of SEQ ID NO: 29, 62, 63 or 64 and/or the other transposon flanking region comprises the nucleotide sequence of SEQ ID NO: 3, 9, 11, 59, 48 or 49 or the nucleotide sequence of SEQ ID NO: 30, 65, 66, 67, 60 or 61. Alternatively the one and the other transposon flanking regions are inverted and the one transposon flanking region comprises the complementary sequences of SEQ ID NO: 3, 9, 11, 59, 48 or 49 or the complementary sequence of SEQ ID NO: 30, 65, 66, 67, 60 or 61 and/or the other transposon flanking region comprises the complementary sequence of SEQ ID NO: 2, 8, 10 or 58 or the complementary sequence of SEQ ID NO: 29, 62, 63 or 64, respectively.

In preferred embodiments, the heterologous polynucleotide comprises at least one sequence selected from the group consisting of a sequence encoding a gene of interest, a complementary DNA (cDNA), a genome of interest and another genetic element.

Further provided is an expression vector comprising the DNA transposon according to the invention.

In another aspect the invention relates to a recombinant transposase comprising at least one heterologous nuclear localization signal (NLS) fused to the transposase, preferably at least a heterologous C-terminal and/or N-terminal NLS, wherein the transposase is an Acyrthosiphon pisum transposase or an Aphis craccivora transposase, and wherein the transposase comprises an amino acid sequence having at least 90% sequence identity with amino acids 10 to 585 of SEQ ID NO: 4.

In a related aspect the invention relates to a recombinant transposase comprising an amino acid sequence having at least 90% sequence identity with amino acids 10 to 585 of SEQ ID NO: 4 and at least one heterologous NLS fused to the transposase. In a preferred embodiment the transposase is an Acyrthosiphon pisum transposase or an Aphis craccivora transposase.

In another related aspect, the invention relates to a recombinant transposase comprising an amino acid sequence having at least 90% sequence identity with amino acids 10 to 585 of SEQ ID NO: 4 and further comprises at least one mutation, preferably wherein at least one heterologous NLS is fused to the transposase. In a preferred embodiment the recombinant transposase according to the invention is hyperactive compared to the transposase of SEQ ID NO: 4. The at least one mutation may be an amino acid substitution selected from the group consisting of K87Y, Q273V, V212I/I215L, 1363V/K365S, K87Y/Q273V, K87Y/A264S/Q273V, A264S/Q273V, S270P/Q273V, K87Y/A264S, L583F, K576I, S372E, S277N and any combination thereof, and/or the at least one mutation is a deletion of N584 and/or E585, wherein the indicated amino acid position of the substitution and/or deletion corresponds to the amino acid position in the amino acid sequence of SEQ ID NO: 4.

Preferably the at least one mutation is an amino acid substitution selected from the group consisting of K87Y, Q273V, V212I/I215L, I363V/K365S, A264S/Q273V, K87Y/A264S, or a combination thereof, and/or the at least one mutation is a deletion of N584 and/or E585.

In yet another aspect, the invention relates to a polynucleotide encoding a transposase comprising an amino acid sequence having at least 90% sequence identity with amino acids 10 to 585 of SEQ ID NO: 4 operably linked to a eukaryotic promoter.

In a related aspect, a polynucleotide encoding the recombinant transposase according to the invention is provided.

The invention further relates to an expression vector encoding the recombinant transposase according to the invention or an expression vector comprising the polynucleotide encoding the transposase according to the invention.

In yet another aspect, the invention relates to an isolated mRNA encoding the recombinant transposase according to the invention.

Also provided is an expression system or a kit comprising (a) a recombinant transposase source selected from the group consisting of (i) the expression vector encoding the recombinant transposase according to the invention; (ii) the isolated mRNA according to the invention and (iii) the recombinant transposase according to the invention; and (b) a DNA transposon according to the invention or an expression vector comprising said DNA transposon according to the invention.

Also provided is a eukaryotic cell comprising the DNA transposon according to the invention. In certain embodiments, the eukaryotic cell is a yeast or a mammalian cell, preferably a mammalian cell, more preferably a rodent or human cell.

Also provided is a non-human transgenic animal comprising the DNA transposon according to the invention.

Also provided is a method for preparing a cell comprising a stably integrated heterologous polynucleotide, comprising (a) introducing a DNA molecule comprising the DNA transposon according to the invention or the expression vector comprising the DNA transposon according to the invention into a eukaryotic cell, wherein the heterologous polynucleotide comprises a sequence encoding a gene of interest, a complementary DNA (cDNA), a genome of interest or another genetic element, and wherein the heterologous polynucleotide further comprises a sequence encoding a selectable marker; (b) introducing a recombinant transposase source into said eukaryotic cell, wherein the recombinant transposase source is selected from the group consisting of: (i) the expression vector encoding the recombinant transposase according to the invention; (ii) the isolated mRNA according to the invention, and (iii) the recombinant transposase according to the invention; and (c) culturing the eukaryotic cell in a medium under conditions to select for the selectable marker, wherein the DNA transposon is stably integrated into the genome of the eukaryotic cell.

Also provided is a method for preparing a protein of interest, comprising (a) introducing a DNA molecule comprising the DNA transposon according to the invention or the expression vector comprising the DNA transposon according to the invention into a eukaryotic cell, wherein the heterologous polynucleotide comprises a sequence encoding at least one protein of interest and further a sequence encoding a selectable marker; (b) introducing a recombinant transposase source into said eukaryotic cell, wherein the recombinant transposase source is selected from the group consisting of: (i) the expression vector encoding the recombinant transposase according to the invention; (ii) the isolated mRNA according to the invention, and (iii) the recombinant transposase according to the invention; and (c) culturing the eukaryotic cell in a medium under conditions to select for the selectable marker, wherein the heterologous polynucleotide comprising a sequence encoding the at least one protein of interest and a sequence encoding a selectable marker is integrated into the genome of the eukaryotic cell, (d) optionally isolating a single clone for clonal expansion to prepare a monoclonal cell line; (e) culturing the eukaryotic cell under conditions to produce the protein of interest; and (f) harvesting and optionally purifying the protein of interest.

### DESCRIPTION OF THE FIGURES

FIG. 1: Plasmid maps. Top: Exemplary plasmid encoding the AP transposase with a heterologous nuclear localization signal (NLS) under the control of a CMV promoter. Bottom: Exemplary transposon vector encoding a heavy and light chain gene of a therapeutic antibody under the control of CMV promoter. A metabolic selection marker (Glutamine Synthetase) is expressed under the control of a SV40 promoter. The transposon cargo is defined with flanking sequences SeqID2 left (Seq ID NO: 2) and SeqID3 right (Seq ID NO: 3). Both Flanking sequences harbour ITR sequences of Seq ID NO:1.

FIG. 2: Selection experiment applying Acyrthosiphon pisum transposase and transposon in CHO (CHO K1 GS^{-/-}) cell deficient of an endogenous glutamine synthetase (GS), with a transposon providing the GS selection marker. As control CHO K1 GS^{-/-} were transfected with the same transposon plasmid, but without the transposase plasmid. Viability is monitored during the selection process of the pool establishment of recombinant CHO cells. Shown is % viability versus day [d] post-transfection.

FIG. 3: Productivity of stable CHO pools transfected with AP transposase and transposon expressing a therapeutic antibody. Control cells were as described for FIG. 2. Shown is the titer [mg/L] versus day [d] post-transfection.

FIG. 4: Copy number determination of genomically integrated heavy chain (HC) and light chain (LC) genes via ddPCR of stable CHO pools transfected with AP transposase and transposon.

FIG. 5: Selection experiment after transfection of various Acyrthosiphon pisum (AP) transposase constructs with and without N- and/or C-terminal heterologous nuclear localization signals (NLS) as indicated and a transposon into CHO-K1 GS−/− cells. Viability was monitored during the selection process of the transfected CHO pools. Shown is % viability versus day [d] post-transfection. AP: wild-type AP transposase (SEQ ID NO: 4), NLS_AP: wild-type AP transposase with heterologous N-terminal NLS (SEQ ID NO: 6); and NLS_AP_NLS: wild-type AP transposase with heterologous N- and C-terminal NLS (SEQ ID NO: 7).

FIG. 6: Antibody titer determination of stable CHO pools 23 days post transfection with various Acyrthosiphon pisum transposase constructs with and without N- and/or C-terminal heterologous nuclear localization signals (NLS) as indicated. Shown is the titer [mg/L] for the different constructs. AP: wild-type AP transposase (SEQ ID NO: 4), NLS_AP: wild-type AP transposase with heterologous N-terminal NLS (SEQ ID NO: 6); and NLS_AP_NLS: wild-type AP transposase with heterologous N- and C-terminal NLS (with flag; SEQ ID NO: 7).

FIG. 7: Selection experiment after transfection of Acyrthosiphon pisum transposase constructs with (NLS_AP_NLS with flag; SEQ ID NO: 7) and without a N-terminal flag-tag (NLS_AP_NLS; SEQ ID NO: 32) and a transposon into CHO-K1 GS−/− cells. Viability was monitored during the selection process of the transfected CHO pools. Shown is % viability versus day [d] post-transfection.

FIG. 8: Titer measurement of stable pools, comparing transposase construct with (SEQ ID NO: 7) and without flag-tag (SEQ ID NO: 32).

FIG. 9: (A) Schematic illustration of tested truncated version of the left and right flanking sequences. (B) Selection experiment after co-transfection of various Acyrthosiphon pisum transposon constructs with varying length of the flanking sequences (left and right) as indicated and AP transposase encoding plasmids. Viability was monitored during the selection of the transfected CHO pools. Shown is % viability versus day [d] post-transfection. (C) Productivity of CHO pools transfected with AP transposase and transposon with varying flanking sequence length as indicated. Shown is the titer [mg/L] versus day [d] post-transfection.

FIG. 10: (A) Sequence alignment of tested left flanking sequences SEQ ID NO: 2, SEQ ID NO: 10 and SEQ ID NO: 12, with the inverted terminal repeats (ITR) core motif (Seq ID NO: 1) in bold, the binding motif (aacac) underlined and the internal repeat (IR) motif (tggtctac) and its inverted complementary sequence in italics and bold. (B) Sequence alignment of tested right flanking sequences SEQ ID NO: 3, Seq ID NO: 11 and Seq ID NO:13, with the ITR core motif (Seq ID NO: 1) in bold, the binding motifs (SEQ ID NO: 50) underlined and the internal repeat (IR) motif (tggtctac) and its inverted complementary sequence in italics and bold.

FIG. 11: (A) Schematic illustration of tested truncated version of the left and right flanking sequences. (B) Selection experiment applying constant full length left flanking sequences (SEQ ID NO: 2) and SEQ ID NO: 3 (WT) or truncated right flanking sequences of the AP transposon (SEQ ID NO: 9, SEQ ID NO: 11 and SEQ ID NO: 13, respectively). Viability [%] is monitored during the selection of the CHO pool. (C) Productivity of CHO pools transfected with constant full length left flanking sequences (SEQ ID NO: 2) and SEQ ID NO: 3 (WT) or truncated right flanking sequences of the AP transposon (SEQ ID NO: 9 and SEQ ID NO: 11, respectively). Productivity (Titer [mg/L]) is monitored at the indicated days post-transfection. (D) Selection experiment applying SEQ ID NO: 2 (WT) or truncated left flanking sequences (SEQ ID NO: 8, SEQ ID NO: 10 and SEQ ID NO: 12, respectively) and constant full length right flanking sequences of the AP transposon (SEQ ID NO: 3). Viability [%] is monitored during the selection of the CHO pool. (E) Productivity of CHO pools transfected with SEQ ID NO: 2 (WT) or truncated left flanking sequences (SEQ ID NO: 8, SEQ ID NO: 10 and SEQ ID NO: 12, respectively) and constant full length right flanking sequences of the AP transposon (SEQ ID NO: 3). Productivity (Titer [mg/L]) is monitored at the indicated days post-transfection. (F) Selection experiment applying truncated left and right flanking sequences of the AP transposon (left: SEQ ID NO: 10 and right: SEQ ID NO: 13 or left SEQ ID NO: 12 and right: SEQ ID NO: 11) compared to wildtype AP transposon (SEQ ID NO: 2 and 3). Viability [%] is monitored during the selection of the CHO pool.

FIG. 12: Selection experiment swapping left (SEQ ID NO: 10) and right (SEQ ID NO: 11) transposon sequences as schematically depicted (top) was analysed by monitoring viability (%) (middle) and productivity (Titer [mg/L]) (bottom) at the indicated days post-transfection.

FIG. 13: (A) Schematic illustration of tested modified version of the left and right flanking sequences. The black, filled symbols represent natural structures, whereas the light, unfilled symbols represent artificially introduced structures; inverted terminal repeats (ITR), inverted internal repeats (IIR), open circle (binding site). (B) Selection experiment applying modified left and right flanking sequences of the AP transposon as indicated. Viability [%] is monitored during the selection of the CHO pools. (C) Productivity of CHO pools transfected with modified left and right flanking sequences of the AP transposon as indicated. Productivity (Titer [mg/L]) is monitored at the indicated days post-transfection.

FIG. 14: Selection experiment after transfection of two different glutamine synthetase selection markers encoding transposons with an AP transposase. Viability is monitored during the selection process of the stable CHO pools. Shown is % viability versus day [d] post-transfection.

FIG. 15: Antibody titers measured at day 21 post transfection using two different glutamine synthetase selection markers encoding transposons with an AP transposase.

FIG. 16: Assessment of fed-batch bioprocess performance of stable recombinant monoclonal antibody expressing CHO cell pools generated using the AP transposase. Three different pools (AP Cell Pool 1-3) derived from different transfection reactions were cultivated in biological duplicates (N=2). (A) Viable cell density [×10⁶ viable cells/ml], (B) cell viability [%] and (C) recombinant monoclonal antibody titer [mg/L] were monitored daily and are shown for the indicated cultivation time [days](determination of antibody titer started at day 9).

FIG. 17: Selection experiment applying functional variants of Acyrthosiphon pisum transposase. Viability is monitored during the selection of the CHO pools. Shown are representative variants, mutant AP K87Y showing a hyperactive phenotype and mutant A264S showing a similar phenotype compared to wild-type AP. Shown is % viability versus day [d] post-transfection.

FIG. 18: Expression oftransmembrane protein NRP1 for bioassays. (A) Gene copy number determination of transfected NRP1 transgene in stable cell pools via ddPCR. Duplicates (Replicate 1: 1^{st} bar; Replicate 2: 2^{nd} bar) of three independent experiments were analysed and are shown as copies/cell. (B) Relative gene expression determination of transfected NRP1 transgene in stable cell pools via ddPCR. Duplicates of three independent experiments were analysed.

FIG. 19: Cell surface staining of expressed NRP1 in stably transfected cell pools analysed by flow cytometry. Histograms show samples stained with anti-NRP1 antibody (“NRP1”, top curves) or isotypic control (“Iso2A”, middle curves) and unstained samples (bottom curves). The bar indicates the gate set for positive staining.

FIG. 20: Activity of Aphis craccivora (AC) transposase and transposon and cross-reactivity of AC and Acyrthosiphon pisum (AP) transposase and transposon. CHO pools transfected with AP/AC transposase and transposon, AC/AP transposase and transposon and AC/AC transposase and transposon were compared to AP/AP transposase and transposon as control for selection behavior and productivity (A) Selection experiment applying functionally different combinations of AC and AP transposon and transposase. Viability [%] is monitored during the selection of the CHO pools. (B) Productivity of CHO pools transfected with AP/AC transposase and transposon and AC/AC transposase and transposon compared to AP/AP transposase and transposon as control. Productivity (Titer [mg/L]) is monitored at the indicated days post-transfection. Sequence alignments of (C) AP transposase (SEQ ID NO: 4) and AC transposase (SEQ ID NO: 26), (D) left transposon flanking region of AP (SEQ ID NO: 2) and AC (SEQ ID NO: 29) and (E) right transposon flanking region of AP (SEQ ID NO: 3) and AC (SEQ ID NO: 30).

FIG. 21: Assessment of fed-batch bioprocess performance of stable recombinant monoclonal antibody expressing CHO cell pools generated using AC transposon/AP transposase (left column; AC/AP), AP transposon/AP transposase (middle column; AP/AP) and AC transposon/AP transposase V212I+I215L+I363V+K365S+Q273V (right column; AC/AP V212I+I215L+I363V+K365S+Q273V). Three different pools derived from different transfection reactions were cultivated in biological duplicates (N=2). (A) Viable cell density [×106 viable cells/ml], (B) cell viability [%], (C) lactate [g/L] and (D) recombinant monoclonal antibody titer [mg/L] were monitored daily and are shown for the indicated cultivation time [days] (determination of antibody titer started at day 6).

FIG. 22: Demonstration of functionality of the AP transposase in human, suspension adapted HEK293F cells using the fluorescent marker zsGreen as transgene and measuring fluorescence by flow cytometry. (A) FACS measurement 8 days after transfection and quantification of zsGreen positive population in host cells alone (host) and with (+) or without (−) AP transposase. (B) FACS measurement 14 days after transfection and quantification of zsGreen positive population in host cells alone (host) and with (+) or without (−) AP transposase. (C) Comparison of % zsGreen positive cells after 8 (top) and 14 days (bottom)+/−AP in separate samples.

FIG. 23: Detection of infectious emGFP encoding adeno-associated virus (AAV) particles produced by either HEK293 or HEK293_Cap_Tet-Rep producer cells transiently transfected with pHelper and pTransfer. Cell culture supernatants from either HEK293 or HEK293_Cap_Tet-Rep producer cellswere transferred to pre-seeded HEK293 cells (96-well plate, 50 μL of cell culture supernatant per well). After 48 hours, emerald green fluorescent protein (emGFP) expression was assessed by fluorescence microscopy using the Cytation 5. The top panels show representative brightfield pictures of a section of cell layer of HEK293 or HEK293_Cap_Tet-Rep producer cells and the bottom panels show GFP expression within the same section. The white scale bar indicates 300 μm. While no AAV-mediated emGFP expression was observed in cells treated with culture supernatant derived from HEK293 cells transfected with pHelper and pTransfer plasmids (which lack Rep and Cap gene expression), emGFP expression was detected in cells treated with the cell culture supernatant derived from stable HEK293_Cap_Tet-Rep cells indicating that the AAV Rep and Cap genes were successfully introduced into HEK293 cells using the AP transposase with AP transposons.

FIG. 24: Selection experiment testing different transposase/transposon-like sequences (Aplysia californica, Onthophagus taurus (variants 1, 2 and 3), Vanessa tameamea and Acyrthosiphon pisum) for transposition activity with the respective transposon flanking regions flanking the GS selectable marker in CHO (CHO K1 GS−/−) cell deficient of an endogenous glutamine synthetase (GS). Viability is monitored during the selection process of the pool establishment of recombinant CHO cells. Shown is % viability versus day [d] post-transfection.

### DETAILED DESCRIPTION

The term “comprises” or “comprising” means “including, but not limited to”. The term is intended to be open-ended, to specify the presence of any stated features, elements, integers, steps or components, but not to preclude the presence or addition of one or more other features, elements, integers, steps, components or groups thereof. The term “comprising” thus includes the more restrictive terms “consisting of” and “essentially consisting of”. With regard to sequences the terms “having an amino acid sequence of” and “comprising an amino acid of” are used interchangeably and include the embodiment “consisting of the amino acid sequence of”. Similarly, the term “encoding” or “encodes” is intended to be open-ended and allows the presence or addition or one or more other features, elements or components. Furthermore, singular and plural forms are not used in a limiting way. As used herein, the singular forms “a”, “an” and “the” designate both the singular and the plural, unless expressly stated to designate the singular only.

The term “encoding” or “encodes” as used herein refers to the sequence or a polynucleotide strand coding for a gene, an RNA and/or a protein, particularly it refers to the coding of the transcribed and/or translated sequence product. It encompasses that a DNA sequence is transcribed into an RNA sequence, in the case of proteins into an mRNA sequence in other cases to a non-coding RNA, such as an siRNA, miRNA or the like. It further encompasses that a mRNA sequence is translated into an amino acid sequence, i.e., a protein. Thus, a gene of interest or a cDNA encodes, e.g., an mRNA or a protein of interest, or a non-coding RNA. A cDNA may also encode a viral genome, as in the case of an RNA virus, such as VSV.

The term “protein” is used interchangeably with “amino acid sequence” or “polypeptide” and refers to polymers of amino acids of any length. These terms also include proteins that are post-translationally modified through reactions that include, but are not limited to, glycosylation, acetylation, phosphorylation, glycation or protein processing. Modifications and changes, for example fusions to other proteins, amino acid sequence substitutions, deletions or insertions, can be made in the structure of a polypeptide while the molecule maintains its biological functional activity. For example, certain amino acid sequence substitutions can be made in a polypeptide or its underlying nucleic acid coding sequence and a protein can be obtained with the same properties.

The term “nucleic acid sequence” is used interchangeably with “polynucleotide” and refers to DNA or RNA of any length. In the context of a DNA transposon or an expression vector, particularly a plasmid, as well as integration into the cell's or host cell's genome the person skilled in the art would understand that it refers to a DNA sequence or molecule.

The term “eukaryotic cell” as used herein refers to cells that have a nucleus within a nuclear envelop and include animal cells, human cells, plant cells and yeast cells. In the present invention a “eukaryotic cell” particularly encompasses mammalian cell, such as Chinese hamster ovary (CHO) cell or HEK293 cell derived cells. Mammalian cells as used herein refer to all cells or cell lines of mammalian origin, such as human or rodent cells. The cells as referred to herein are cells maintained in culture, such as cell lines or cell line derived cells, i.e., immortalized cells, or primary cells ex vivo. Primary cells are cells isolated from organ tissue or an organism and maintained in vitro for growth and/or adoptive cell transfer into a patient. For adoptive cell transfer in a patient (preferably a human patient), the primary cell is preferably a patient autologous cell.

The term “about” as used herein refers to a variation of 10% of the value specified, for example, about 50% carries a variation from 45 to 55%.

The term “selection stringency” as used herein refers to the duration to reach more than 70% viability and a doubling time of 48 h or less of the cell culture following transfection. The longer the time period, the more stringent the selection behavior. For example, an attenuated glutamine synthetase shows a more stringent selection behavior compared to CHO wildtype glutamine synthetase.

The term “transposon flanking region” as used herein relates to a sequence flanking a heterologous polynucleotide. The left and right transposon flanking region together with the heterologous polynucleotide form the DNA transposon having one end (e.g., a left (5′) end) and another end (e.g., a right (3′) end) marked by the target sequence. Each transposon flanking region comprises a target sequence (e.g., TTAA), an inverted terminal repeat (ITR) and typically further a binding motif and a further sequence between the ITR/binding motif and the heterologous polynucleotide comprising at least one inverted internal repeat (IIR) and optionally at least one further binding motif, preferably adjacent to the IIR. The at least one IIR may be directly adjacent to the ITR/binding motif and/or the IIR may be separated from the ITR/binding motif.

The term “DNA transposon” as used herein relates to a heterologous polynucleotide flanked by a left and a right transposon flanking region, wherein the transposon has a target sequence (e.g., TTAA) at each end followed (left transposon flanking region) or preceded (right transposon flanking region) by an ITR. Thus, the one transposon end (such as left or 3′ of the heterologous polynucleotide) comprises a target sequence followed by an ITR and the other transposon end (such as right or 5′ of the heterologous polynucleotide) comprises an ITR followed by a target sequence. The sequence encoding the target sequence/ITR (e.g., SEQ ID NO: 38 and 39) therefore mark the two ends of the transposon. The DNA transposon may also be referred to as transposon herein.

The term “heterologous polynucleotide” as used herein refers to a sequence heterologous to the transposon flanking region, i.e., not derived from the same organism and/or the same gene location as the transposon flanking region, and encompasses any recombinant polynucleotide. The term “recombinant” as used herein refers to a DNA molecule formed by methods of genetic recombination (such as cloning) that bring together genetic material from multiple sources or introducing mutations. A heterologous polynucleotide or a recombinant polynucleotide may comprise a gene of interest, a complementary DNA (cDNA, such a encoding a protein of interest or a viral genome of interest), a genome of interest (such as a viral genome of interest) or any other genetic element. Although the recombinant polynucleotide may also encode a recombinant transposase heterologous to the transposon flanking region, in certain embodiments of the present invention, the heterologous polynucleotide is not a polynucleotide encoding a transposase from Acyrthosiphon pisum or even a polynucleotide encoding a transposase.

The term “transposase” as used herein is a class of enzymes capable of binding to the end of a transposon and catalyzing its movement to another part of a genome in cis (i.e., within a genome) and in trans (e.g., from an expression vector, such as a plasmid, to a genome).

The term “transposase/transposon-like sequences” as used herein refers to sequences comprising elements characteristic for transposases and transposons, such as repetitive, ITR-like elements 3′ and 5′ of a potential transposase sequence and potential transposase sequences comprising catalytic amino acid residues (DDE-motif), without referring to its functional activity in cells. For example, US 2020/0318107 A1 discloses that PiggyBac-like transposases and transposons occur naturally in a wide range of organisms, including Acyrthosiphon pisum (XP_001948139) and mentions that transposition activity has been described for almost none of these. Although the transposase according to the present invention is also of the PiggyBac type, it differs significantly from the sequence of the Acyrthosiphon pisum transposase (XP_001948139) referred to in US 2020/0318107 A1 and the AP transposase gene of the present invention only shares about 21% sequence identity therewith. By contrast, active transposase/transposon sequences are referred to herein as (active) transposase/(DNA) transposon pair or system.

The term “nuclear localization signal (NLS)” as used herein refers to a short peptide that acts as a signal fragment that mediates the transport of a protein from the cytoplasm into the nucleus. NLS are reviewed by Lu et al. (Cell Commun Signal (2021) 19: 60), which is incorporated herein by reference. In brief classical nuclear localization signals (cNLS) encompass two categories, termed “monopartite” (MP) and “bipartite” (BP). MP NLS are a single cluster composed of 4-8 basic amino acids, which generally contain 4 or more positively charged residues (arginine (R) or lysine (K)). The characteristic motif of MP NLS is K (K/R) X (K/R), wherein X can be any residue. For example, the NLS of SV40 large T-antigen is PKKKRKV (SEQ ID NO: 5), with 5 consecutive positively charged amino acids. By contrast, BP NLS are characterized by two clusters of 2-3 positively charged amino acids that are separated by a 9-12 amino-acid linker region, which contains several proline (P) residues. The consensus sequence can be expressed as R/K(X)10-12KRXK. For example the C-terminal NLS of nucleoplasmin is KRPAATKKAGQAKKKK (SEQ ID NO: 14). Non-classical NLS (ncNLS) are neither similar to canonical signals nor rich in arginine or lysine residues, such as the “proline-tyrosine” category, named PY-NLS.

The term “% sequence identity” as used herein refers to the number of characters that match exactly between two different sequences in a sequence alignment, in the case of a nucleotide sequence the bases that match exactly and in the case of an amino acid sequence the amino acids that match exactly.

The term “fusion protein” as used herein refers to a protein generated by joining two or more genes in frame that originally coded for separate proteins or protein fragments. A fusion protein may also be referred to as chimeric protein. A fusion protein contains an Fc-fusion protein and the like, but is not limited thereto. The term “fused” similarly describes that two proteins or protein fragments of originally separate proteins are joined, i.e., fused, together to form a single protein. It particularly includes the introduction of a heterologous nuclear localization signal into the sequence of the transposase according to the invention. The term introduction of a heterologous nuclear localization signal means introduction of the heterologous NLS in addition and/or to substitute a native putative nuclear localization signal. The heterologous NLS may be any NLS different to the native putative NLS of the transposase according to the invention (e.g. SEQ ID NO: 4 or SEQ ID NO: 26).

### DNA Transposon and Polynucleotides or Expression Vectors Encoding Said DNA Transposon

The present invention provides a novel transposase/transposon pair, e.g. from Acyrthosiphon pisum (AP) or Aphis craccivora (AC), to transpose polynucleotides encoding a variety of different proteins or non-coding RNA into a cell for stable expression by integration into the cells genome. These novel transposase/transposon pairs present additional, highly efficient orthogonal transposase/transposon pairs, that are functionally active in eukaryotic cells and with a transposition efficacy that is at least comparable with commercially available transposase/transposon systems. The transposase according to the invention is specifically recognizing the identified transposon ends comprising the inverted terminal repeats (ITRs), which are substantially different from any published transposase/transposon system, and transposition leads to the stable integration of any cargo polynucleotide into the cell's genome. The transposase/DNA transposon systems(s) according to the present invention is/are Piggy-Bac transposase/transposon pairs.

More specifically, in a one aspect, the invention relates to a DNA transposon comprising a heterologous polynucleotide flanked by transposon flanking regions, wherein one transposon flanking region comprises the nucleotide sequence of SEQ ID NO: 38 at one transposon end; and the other transposon flanking region comprises the nucleotide sequence of SEQ ID NO: 39 at the other transposon end. The sequence of SEQ ID NO: 38 consists of the target sequence TTAA and the inverted terminal repeat sequence (SEQ ID NO: 1) and the sequence of SEQ ID NO: 39 consists of the inverted terminal repeat sequence (SEQ ID NO: 15) and the target sequence TTAA. The nucleotide sequences of SEQ ID NOs: 38 and 39, as well as the nucleotide sequences of SEQ ID NOs: 1 and 15, are inverse complementary to each other. Yet, the one and the other transposon flanking regions of the DNA transposon according to the invention are preferably not inverse complementary to each other over the entire length of the transposon flanking region. The nucleotide sequences of SEQ ID NO: 38 and 39 mark the end of the one and the other transposon flanking regions. Thus, for example the nucleotide sequence of SEQ ID NO: 38 is at the left end (beginning of) the left transposon flanking region and the nucleotide sequence of SEQ ID NO: 39 is at the right end (end of) the right transposon flanking region, i.e., forming and marking the ends of the DNA transposon. The left and the right transposon flanking region may also be referred to as the 3′ and the 5′ transposon flanking region, respectively, and describes the sequence upstream and downstream of the heterologous polynucleotide, respectively. However, the person skilled in the art will understand that the 3′ and 5′ end will be swapped by inversion of the entire polynucleotide comprising the DNA transposon. Moreover, the person skilled in the art will understand that the one and the other transposon flanking regions can be used as the left and the right transposon flanking region or alternatively as the right and the left transposon flanking region comprising the inverted complementary sequence. Particularly, the transposon according to the present invention is transposable by a transposase comprising the amino acid sequence of SEQ ID NO: 4.

In a preferred embodiment the one transposon flanking region comprises the nucleotide sequence of SEQ ID NO: 40 at one transposon end; and the other transposon flanking region comprises the nucleotide sequence of SEQ ID NO: 41 at the other transposon end. The nucleotide sequence of SEQ ID NO: 40 comprises the nucleotide sequence of SEQ ID NO: 38 and further the binding motif aggcgcg (SEQ ID NO: 50) and the nucleotide sequence of SEQ ID NO: 41 comprises the binding motif aggcgcg (SEQ ID NO: 50) and further the nucleotide sequence of SEQ ID NO: 39, wherein the binding motif is overlapping with the ITR sequences. The one and the other transposon flanking region preferably further comprises at least one inverted internal repeat (IIR) comprising (i) an internal repeat (IR) motif comprising the sequence tggtctac and (ii) its reverse complementary sequence, and wherein (iii) the IR and its reverse complementary sequence are separated by three to six, preferably four nucleotides. In certain embodiments, the IR motif consists of the sequence tggtctac. Preferably, the four nucleotides separating the IR tggtctac and its reverse complementary sequence are selected from the group consisting of AATT, AATC, AACT, AAGT and AGGC. The internal repeat motif and its reverse complementary sequence separated by three to six, preferably four nucleotides is referred to as inverted internal repeat herein. In certain embodiments the at least one inverted internal repeat (IIR) motif has the nucleotide sequence of SEQ ID NO: 43 or 44. The term “inverted internal repeat (IIR)” as used herein refers to a nucleotide sequence of an internal repeat (IR) motif followed downstream by its reverse complement with intervening nucleotides between the initial internal repeat motif and the reverse complement, wherein the inverted internal repeat is located between the inverted terminal repeat and the heterologous polynucleotide. The IR motif of the transposon of the present invention has the nucleotide sequence tggtctac (SEQ ID NO: 42) and three to six, preferably four to five, more preferably four intervening nucleotides between the initial internal repeat motif and the reverse complement. The intervening nucleotides can be any nucleotide (i.e., a, c, g or t), preferably the first intervening nucleotide is an adenosine. Preferably, the intervening nucleotides are four intervening nucleotides selected from the group consisting of AATT, AATC, AACT, AAGT and AGGC. More preferably the four intervening nucleotides in the at least one IIR of the one transposon flanking region and the other transposon flanking region are different, even more preferably the four intervening nucleotides in the at least one IIR of the one transposon flanking region and the other transposon flanking region are different and in case of more than one IIR in the one or the other transposon flanking region, the four intervening nucleotides of the IIRs within the same transposon flanking region are different. Thus, the IIR motif of the transposon of the present invention preferably has the nucleotide sequence of SEQ ID NO: 43, more specifically of SEQ ID NO: 44. In order to increase the sequence variation between the one and the other transposon flanking region, the four intervening nucleotides in the at least one IIR of the one transposon flanking region and the other transposon flanking region are preferably different, more preferably the four intervening nucleotides in the at least one IIR of the one transposon flanking region and the other transposon flanking region each are different, i.e., in case of more than one IIR in the one or the other transposon flanking region each of the IIR within the same transposon flanking region are further different. The transposon may further comprise a second binding motif cgcgcct, wherein the binding motif may be adjacent to or overlapping with the IIR, preferably the binding motif is adjacent to the IIR.

The at least one IIR of the one and the other transposon flanking region may independently be directly adjacent to the binding motif of the ITR or may be separated by a strand of nucleotides from the binding motif of the ITR, preferably by about 10 to 50 nucleotides, more preferably about 20 to 40 nucleotides, even more preferably about 25 to 35 nucleotides. Preferably at least one IIR of the one transposon flanking region is directly adjacent to the binding motif of the ITR and the at least one IIR of the other transposon flanking region is separated by a strand of nucleotides from the binding motif of the ITR. The one and/or the other transposon flanking region may also comprise more than one IIR, such as two or three IIR, preferably two IIR. For example, the one transposon flanking region may comprise two IIRs and the other transposon flanking region comprises one IIR (or vice versa). In the case of two IIRs, the first IIR is preferably adjacent to the ITR and binding region and the second IIR is separated by about 25 to 250 nucleotides from the first IIR, preferably by about 25 to 200 nucleotides, more preferably about 50 to 200 nucleotides, even more preferably 120 to 160 nucleotides. In the case of one IIR, the IIR is preferably separated by about 10 to 50 nucleotides, more preferably about 20 to 40 nucleotides, even more preferably about 25 to 35 nucleotides from the binding motif (cgcgcct) of the of the ITR, wherein the IIR preferably further comprises a binding motif (cgcgcct) or a binding motif-like motif (agcgcct). Thus, in certain embodiments the IIR-binding motif has the sequence TGGTCTACAnnnGTAGACCAmGCGCCT (SEQ ID NO: 68), preferably TGGTCTACAnnnGTAGACCACGCGCCT (SEQ ID NO: 69) or TGGTCTACAnnnGTAGACCAAGCGCCT (SEQ ID NO: 70), more preferably SEQ ID NO: 70. The person skilled in the art would understand that the one and the other transposon flanking region may also be used in inverted orientation (such as in the respective inverse complementary sequences).

In preferred embodiments, the one transposon flanking region comprises the nucleotide sequence of SEQ ID NO: 45 or 46 and further an inverted internal repeat (IIR) motif having the nucleotide sequence of SEQ ID NO: 43 separated by about 50 to 200 nucleotides, preferably wherein the one transposon flanking region comprises a sequence having 85% sequence identity with SEQ ID NO: 10 or 63, or with SEQ ID NO: 2 or 29; and/or the other transposon flanking region comprises the nucleotide sequence of SEQ ID NO: 47, or a sequence having at least 95% sequence identity with SEQ ID NO: 47, wherein the nucleotide sequence comprises at least the nucleotide sequence of SEQ ID NO: 41 and the nucleotide sequence of SEQ ID NO: 43, preferably at least the nucleotide sequence of SEQ ID NO: 41 and the nucleotide sequence of SEQ ID NO: 68, 69 or 70. In a more specific embodiment the other transposon flanking region comprises a sequence having at least 97%, preferably 98%, more preferably 99% sequence identity with SEQ ID NO: 47, wherein the nucleotide sequence comprises at least the nucleotide sequence of SEQ ID NO: 41 and the nucleotide sequence of SEQ ID NO: 43, preferably at least the nucleotide sequence of SEQ ID NO: 41 and the nucleotide sequence of SEQ ID NO: 68, 69 or 70. In certain embodiments, the other transposon flanking region comprises the nucleotide sequence of SEQ ID NO: 48, 49, 60 or 61. The nucleotide sequence of SEQ Id NO: 45 or 46 comprises the nucleotide sequence of SEQ ID NO: 38, further the binding motif aggcgcg (SEQ ID NO: 50) and a first IIR. Thus, the IIR further to the nucleotide sequence of SEQ ID NO: 45 or 46 is a second IIR. Preferably the one transposon flanking region is the upstream (left or 5′) transposon flanking region and the other transposon flanking region is the downstream (right or 3′) transposon flanking region. Alternatively, the one and the other transposon flanking regions may be inverted or switched (i.e., the one transposon flanking region is the downstream transposon flanking region and the other is the upstream transposon flanking region), because both transposon flanking regions may be used as upstream or downstream transposon flanking region. Thus, in an alternative preferred embodiment the one and the other transposon flanking regions are inverted and comprise reverse complementary sequences of the sequences recited above, i.e., of a nucleotide sequence of SEQ ID NO: 45 or 46 and further an inverted internal repeat (IIR) motif having the nucleotide sequence of SEQ ID NO: 43 separated by about 50 to 200 nucleotides for the one transposon flanking region and the nucleotide sequence of SEQ ID NO: 47, and/or a sequence having at least 95% sequence identity with SEQ ID NO: 47, wherein the sequence comprises at least the nucleotide sequence of SEQ ID NO: 41 and the nucleotide sequence of SEQ ID NO: 43 for the other transposon flanking region, preferably at least the nucleotide sequence of SEQ ID NO: 41 and the nucleotide sequence of SEQ ID NO: 68, 69 or 70.

In a more specific embodiment, (a) the one transposon flanking region comprises a sequence having at least 85% nucleotide sequence identity with SEQ ID NO: 2, 8, 10 or 58 (preferably SEQ ID NO: 2 or 10, more preferably SEQ ID NO: 2), and/or the other transposon flanking region comprises a sequence having at least 90%, preferably at least 95% nucleotide sequence identity with SEQ ID NO: 3, 9, 11 or 59 (preferably SEQ ID NO: 3 or 11, more preferably SEQ ID NO: 3); or alternatively (b) the one and the other transposon flanking regions are inverted and comprise reverse complementary sequences of the sequences recited above, i.e., of a sequence having at least 85% nucleotide sequence identity with SEQ ID NO: 2, 8, 10 or 58 and/or a sequence having at least 90%, preferably at least 95% nucleotide sequence identity with SEQ ID NO: 3, 9, 11, 59, 48 or 49. Preferably the one transposon flanking region comprises a sequence having at least 90%, preferably at least 95%, at least 97%, at least 98% at least 99% or 100% nucleotide sequence identity with SEQ ID NO: 2, 8, 10 or 58 and/or the other transposon flanking region comprises a sequence having at least 95%, preferably at least 97%, at least 98%, at least 99% or 100% nucleotide sequence identity with SEQ ID NO: 3, 9, 11, 59, 48 or 49. Thus, in certain embodiments, the one transposon flanking region comprises the nucleotide sequence of SEQ ID NO: 2, 8, 10 or 58 and/or the other transposon flanking region comprises the nucleotide sequence of SEQ ID NO: 3, 9, 11, 59, 48 or 49, or the one and the other transposon flanking regions are inverted and comprise reverse complementary sequences thereof.

It was further found that the left and right transposon flanking regions of Aphis craccivora (SEQ ID NOs: 29 and 30, respectively), which share 85.7% and 95.8% sequence identity with the left and right transposon flanking regions of Acyrthosiphon pisum (SEQ ID NO: 2 and 3, respectively) were equally functional (selection behavior and productivity) when used in combination with their respective wild-type transposase (AC transposase: SEQ ID NO: 26 and AP transposase: SEQ ID NO: 4). Thus, the related transposase-transposon system from AC is also a transposase-transposon system according to the present invention. The AC left and right transposon flanking regions are further functional when used in combination with the AP transposase. Thus, the AP transposase/AC transposon system Is also a transposase/DNA transposon system according to the invention. In a more specific embodiment, (a) the one transposon flanking region comprises a sequence having at least 85% nucleotide sequence identity with SEQ ID NO: 2, 8, 10 or 58 (preferably SEQ ID NO: 2 or 10, more preferably SEQ ID NO: 2) or with SEQ ID NO: 29, 62, 63 or 64 (preferably SEQ ID NO: 29 or 63, more preferably SEQ ID NO: 29), and/or the other transposon flanking region comprises a sequence having at least 90%, preferably at least 95% nucleotide sequence identity with SEQ ID NO: 3, 9, 11, 59, 48 or 49 (preferably SEQ ID NO: 3 or 11, more preferably SEQ ID NO: 3) or with SEQ ID NO: 30, 65, 66, 67, 60 or 61 (preferably SEQ ID NO: 30 or 66, more preferably SEQ ID NO: 30); or alternatively (b) the one and the other transposon flanking regions are inverted and comprise reverse complementary sequences of the sequences recited above, i.e., of a sequence having at least 85% nucleotide sequence identity with SEQ ID NO: 2, 8, 10 or 58 or with SEQ ID NO: 29, 62, 63 or 64 and/or a sequence having at least 90%, preferably at least 95% nucleotide sequence identity with SEQ ID NO: 3, 9, 11, 59, 48 or 49 or with SEQ ID NO: 30, 65, 66, 67, 60 or 61. Preferably the one transposon flanking region comprises a sequence having at least 90%, preferably at least 95%, at least 97%, at least 98% at least 99% or 100% nucleotide sequence identity with SEQ ID NO: 29, 62, 63 or 64 and/or the other transposon flanking region comprises a sequence having at least 95%, preferably at least 97%, at least 98%, at least 99% or 100% nucleotide sequence identity with SEQ ID NO: 30, 65, 66, 67, 60 or 61. Thus, in certain embodiments, the one transposon flanking region comprises a sequence of SEQ ID NO: 29, 62, 63 or 64 and/or the other transposon flanking region comprises a sequence of SEQ ID NO: 30, 65, 66, 67, 60 or 61; or the one and the other transposon flanking regions are inverted and comprise reverse complementary sequences thereof.

The person skilled in the art will understand that the nucleotide sequence of the left transposon flanking region of AP of SEQ ID NO: 2 is the full length sequence, while the nucleotide sequences of SEQ ID NO: 8, 10 or 58 are truncated forms thereof comprising the minimal essential elements and the nucleotide sequence of the left transposon flanking region of AC of SEQ ID NO: 29 is the full length sequence, while the nucleotide sequences of SEQ ID NO: 62, 63 or 64 are truncated forms thereof comprising the minimal essential elements. Thus, in certain embodiments, (i) the one transposon flanking region comprises a sequence having at least 85% (or at least 90%, at least 95%, at least 97%, at least 98% at least 99% or 100%) nucleotide sequence identity with at least SEQ ID NO: 58, preferably with at least SEQ ID NO: 10, at least SEQ ID NO: 8 or SEQ ID NO: 2; or comprises a sequence having at least 85% (or at least 90%, at least 95%, at least 97%, at least 98% at least 99% or 100%) nucleotide sequence identity with at least SEQ ID NO: 64, preferably with SEQ ID NO: 63, SEQ ID NO: 62 or SEQ ID NO: 29; and/or the other transposon flanking region comprises a sequence having at least 90% (at least 95%, preferably at least 97%, at least 98%, at least 99% or 100%) sequence identity with at least SEQ ID NO: 48 or 49, preferably with at least SEQ ID NO: 59, at least SEQ ID NO: 11, SEQ ID NO: 9 or SEQ ID NO: 3, or comprises a sequence having at least 90% (at least 95%, preferably at least 97%, at least 98%, at least 99% or 100%) sequence identity with SEQ ID NO: 60 or 61, preferably with at least SEQ ID NO: 67, at least SEQ ID NO: 66, at least SEQ ID NO: 65 or SEQ ID NO: 30, or (ii) wherein the one and the other transposon flanking regions are inverted and comprise reverse complementary sequences of the sequences recited in (i).

Preferably, (i) the one transposon flanking region comprises a sequence having at least 85% (or at least 90%, at least 95%, at least 97%, at least 98% at least 99% or 100%) nucleotide sequence identity with at least SEQ ID NO: 64, preferably with at least SEQ ID NO; 63, at least SEQ ID NO: 62 or SEQ ID NO: 29; and/or the other transposon flanking region a sequence having at least 90% (at least 95%, preferably at least 97%, at least 98%, at least 99% or 100%) sequence identity with at least SEQ ID NO: 60 or 61, preferably with at least SEQ ID NO: 67, at least SEQ ID NO: 66, at least SEQ ID NO: 65 or more preferably SEQ ID NO: 30, or (ii) wherein the one and the other transposon flanking regions are inverted and comprise reverse complementary sequences of the sequences recited in (i). The full-length and truncated sequences of the left and right transposon flanking regions of AP and AC are summarized in Table A below:

TABLE A

| | AP | AC | AP | AC |
|---|---|---|---|---|
| | Left transposon flanking region | | Right transposon flanking region | |
| Full-length | SEQ ID NO: 2 | SEQ ID NO: 29 | SEQ ID NO: 3 | SEQ ID NO: 30 |
| | (340 nt) | (362 nt) | (415 nt) | (407 nt) |
| Truncated | SEQ ID NO: 8 | SEQ ID NO: 62 | SEQ ID NO: 9 | SEQ ID NO: 65 |
| | (297 nt) | (320 nt) | (260 nt) | (260 nt) |
| Truncated | SEQ ID NO: 10 | SEQ ID NO: 63 | SEQ ID NO: 11 | SEQ ID NO: 66 |
| | (214 nt) | (225 nt) | (154 nt) | (154 nt) |
| | | | SEQ ID NO: 59 | SEQ ID NO: 67 |
| | | | (77 nt) | (77 nt) |
| Truncated | SEQ ID NO: 58 | SEQ ID NO: 64 | SEQ ID NO: 48/49 | SEQ ID NO: 60/61 |
| | (200 nt) | (211 nt) | (67 nt) | (67 nt) |
| Truncated* | SEQ ID NO: 12 | | SEQ ID NO: 13 | |
| | (86 nt) | | (57 nt) | |

*non-functional, lacks essential IIR

The person skilled in the art will understand that each of the one and the other transposon flanking region may be independently an AP derived sequence or an AC derived sequence, preferably the one and the other transposon flanking region are both AP derived sequences or are both AC derived sequences, more preferably the one and the other transposon flanking region are both AC derived sequences (including the respective recited % sequence identities). For example, without being limited thereto, AC derived sequences are sequences having at least 95% sequence identity with SEQ ID NO: 29, 62, 63 or 64 for the one transposon flanking region and sequences having at least 97% sequence identity with SEQ ID NO: 30, 65, 66, 67, 60 or 61 for the other transposon flanking region (or the reverse complementary sequences thereof), and AP derived sequences are sequences having at least 95% sequence identity with SEQ ID NO: 2, 8, 10, or 58 for the one transposon flanking region and sequences having at least 97% sequence identity with SEQ ID NO: 3, 9, 11, 59, 48 or 49 for the other transposon flanking region (or the reverse complementary sequences thereof). For the left transposon flanking regions, at least SEQ ID NOs: 10, 8 and 2 (or SEQ ID NOs: 63, 62 and 29) are equally preferred, while for the right transposon flanking region function increases with length, and hence SEQ ID NO: 9 or 3 (or SEQ ID NO: 65 or 30) are preferred and SEQ ID NO: 3 (or SEQ ID NO: 30) is more preferred.

In certain preferred embodiments (i) the one transposon flanking region comprises a sequence having at least 95% (preferably at least 97%, at least 98%, at least 99% or at least 100%) sequence identity with SEQ ID NO: 29, 62 or 63 and/or the other transposon flanking region comprises a sequence having at least 97% (preferably at least 98%, at least 99% or at least 100%) sequence identity with SEQ ID NO: 30, 65 or 66, preferably SEQ ID NO: 30; or (ii) wherein the one and the other transposon flanking regions are inverted and comprise reverse complementary sequences of the sequences recited in (i).

In the DNA transposon according to the invention the transposon flanking regions flank a heterologous polynucleotide that is a polynucleotide of interest. The person skilled in the art would understand that the heterologous polynucleotide of interest is different to the natural occurring sequence flanked by the transposon flanking regions. Thus, the polynucleotide of interest is not a of the family of Aphididae or the suborder of Sternorrhynca. In the DNA transposon according to the invention the transposon flanking regions flank a heterologous polynucleotide comprising at least one sequence selected from the group consisting of a sequence encoding (or comprising) a gene of interest, a complementary DNA (cDNA), a genome of interest and another genetic element. A gene of interest or a cDNA may encode a protein of interest or a non-coding RNA (ncRNA). The term “non-coding RNA” as used herein refers to an RNA molecule that is not translated into a protein. The DNA from which a functional non-coding RNA is transcribed may also be referred to as RNA gene. Non-coding RNAs (ncRNAs) include, without being limited thereto, transfer RNA (tRNA), ribosomal RNA (rRNA), as well as small RNAs (such as microRNA (miRNA), siRNA, shRNA, piRNA, snoRNA, snRNA), long non-coding RNA (lncRNA), anti-sense RNAs, riboswitches and ribozymes. The protein of interest is preferably a recombinant protein and includes without being limited thereto a therapeutic protein, particularly a secreted recombinant therapeutic protein (such as an antibody, an antibody-derived molecule or an antibody mimetic, a cytokine, a hormone, a fusion protein and the like), a transmembrane receptor (e.g., for overexpression in a cell for use in an in vitro assay or a chimeric antigen receptor (CAR) or a cytokine receptor for engineering patient autologous cells, such as T cells or NK cells, for adoptive cell transfer) and an enzyme (e.g., for genetic engineering of a protein production cell, such as a glycosylation modifying enzyme). The transposon of the present invention may also be used for viral production (such as AAV or VSV) or the generation of a virus packaging cell line. Thus, the protein of interest may be also a viral protein, such as an AAV viral protein, preferably encoded by the AAV rep and/or cap gene.

The transposon according to the invention is not limited in size and may comprise a heterologous polynucleotide as large as 40 or 50 kb. Thus, the heterologous polynucleotide may therefore also comprise a sequence comprising a genome of interest, e.g., a viral genome of interest, such as the viral genome of adeno-associated virus (AAV) or vesicular stomatitis virus (VSV), or one or more viral genes, such as from AAV (rep and/or cap) or VSV. The AAV genome is flanked by two inverted terminal repeats (referred to as viral inverted terminal repeats in the following to distinguish over the ITRs in the transposon according to the invention). The entire AAV genome comprises about 4.7 kb including the viral ITRs. In the context of the present invention the AAV genome is typically a recombinant AAV (rAAV) genome that may encode viral proteins or proteins heterologous thereto, such as a therapeutic protein or a suicide gene product. Typically for RNA viruses, such as VSV, the viral genome is encoded by a genomic cDNA. Thus, cDNA may encode for a viral genome, a protein of interest or a non-coding RNA. The transposon according to the invention may further be used for stable integration of other genetic elements, such as a binding motif or a regulatory element.

In certain embodiments, the heterologous polynucleotide comprises at least one sequence selected from the group consisting of a sequence encoding (or comprising) a gene of interest, a complementary DNA (cDNA), a genome of interest and another genetic element, wherein the heterologous polynucleotide comprises the at least one sequence under the control of a promoter and a transcription termination signal. The promoter may be any promoter compatible with the host cell. Preferably the host cell is a eukaryotic host cell. In certain embodiments, the promoter is a eukaryotic promoter, preferably selected from the group consisting of an EF1a promoter, a cytomegalovirus (CMV) promoter, a GAPDH promoter, a CAG promoter, a Herpes Simplex Virus thymidine kinase (HSV-TK) promoter, a Murine Stem Cell Virus (MSCV) promoter, a spleen focus-forming virus (SFFV) promoter, an SV40 promoter and an actin promoter, a PGK promoter and an ubiquitin promoter. In certain or additional embodiments the promoter is an inducible promoter, e.g. comprising a Tet-regulatory element. The transcription termination signal is typically a polyadenylation site. Preferably, the heterologous polynucleotide comprises an expression cassette comprising a promoter, at least one the at least one sequence selected from the group consisting of a sequence encoding a gene of interest, a complementary DNA (cDNA), a genome of interest and another genetic element and a transcription termination signal. More preferably, the heterologous polynucleotide comprises an expression cassette comprising a promoter, at least one sequence encoding a gene of interest, a cDNA or a genome of interest and a transcription termination signal.

In certain embodiments the heterologous polynucleotide comprises at least one sequence selected from the group consisting of a sequence or sequences encoding a heavy and/or a light chain of an antibody; a secreted therapeutic recombinant protein; a recombinant protein; a transmembrane receptor; a non-coding RNA mediating RNA interference (RNAi), preferably selected from the group consisting of siRNA, shRNA, lncRNA and miRNA; one or more viral proteins; a viral genome or a viral genomic cDNA; a ribozyme; a binding motif; a regulatory DNA element, another genetic element; and a combination of any one of the above.

The protein of interest encoded by the gene of interest may be any protein, but is typically a therapeutic protein. The term “therapeutic protein” as used herein refers to proteins that can be used in medical treatment of humans and/or animals. These include, but are not limited to cytokines, growth factors, hormones, blood coagulation factors, vaccines, interferons, fusion proteins, antibodies, antibody-derived molecules and an antibody mimetics. In certain embodiments, the therapeutic protein is selected from the group consisting of a cytokine, a hormone, a fusion protein, an antibody, an antibody-derived molecule and an antibody mimetic.

In certain embodiments the protein of interest is an antibody. In cases where the protein of interest is an antibody, the DNA transposon or the eukaryotic expression vector (particularly the mammalian expression vector) comprises a polynucleotide comprising a coding sequence for a variable region of the heavy chain and/or a coding sequence for a variable region of the light chain of the antibody. In certain embodiments, DNA transposon comprises a polynucleotide comprising a coding sequence for a heavy chain and/or a coding sequence for a light chain of the antibody. Thus, the polynucleotide comprising a coding sequence for a variable region of the heavy chain and the polynucleotide comprising a coding sequence of a variable region of the light chain may be expressed by the same DNA transposon or by separate DNA transposons. The DNA transposon may comprise a multicistronic expression cassette, such as a bicistronic expression cassette, and/or multiple expression cassettes. A multicistronic expression cassette comprises more than one open reading frames separated by sequences coding for an RNA element that allows for translation initiation, such as an internal ribosomal entry site (IRES). In a multicistonic expression cassette, the two or more open reading frames are under the control of the same promoter. The polynucleotide encoding at least a variable region of the heavy chain and the polynucleotide encoding at least a variable region of the light chain may therefore be expressed within the same expression cassette (separated e.g., by an IRES sequence) or by two separate expression cassettes. Moreover, the selectable marker and the protein of interest and/or the non-coding RNA may be expressed by the same or separate expression cassette(s). In case the protein of interest is an antibody, the selectable marker (e.g., glutamine synthetase) gene, the polynucleotide encoding at least a variable region of the heavy chain and/or the polynucleotide encoding at least a variable region of the light chain may be expressed by the same or separate expression cassettes or a mixture thereof.

In preferred embodiments, the DNA transposon comprises the polynucleotide encoding the selectable marker (e.g., glutamine synthetase) and the at least one sequence encoding a gene of interest, a cDNA, a genome of interest or another genetic element. The eukaryotic expression vector comprising the DNA transposon according to the invention, preferably the mammalian expression vector according to the invention, is preferably a plasmid, a Bacterial Artificial Chromosome (BAC) or a non-integrating viral vector. Said plasmid, Bacterial Artificial Chromosome (BAC) or viral vector may be introduced into the eukaryotic host cell (such as the mammalian host cell) via transfection or transduction, respectively.

A preferred protein of interest is an antibody, including fragments and derivatives thereof. Typically, an antibody is monospecific, but an antibody may also be multispecific. Thus, the present invention may be used for the production of mono-specific antibodies, multi-specific antibodies, or fragments thereof, preferably of antibodies (mono-specific), bispecific antibodies, trispecific antibodies or fragments thereof, preferably antigen-binding fragments thereof. Exemplary antibodies within the scope of the present invention include but are not limited to anti-CD2, anti-CD3, anti-CD20, anti-CD22, anti-CD30, anti-CD33, anti-CD37, anti-CD40, anti-CD44, anti-CD44v6, anti-CD49d, anti-CD52, anti-EGFR1 (HER1), anti-EGFR2 (HER2), anti-GD3, anti-IGF, anti-VEGF, anti-TNFalpha, anti-IL2, anti-IL-5R or anti-IgE antibodies, and are preferably selected from the group consisting of anti-CD20, anti-CD33, anti-CD37, anti-CD40, anti-CD44, anti-CD52, anti-HER2/neu (erbB2), anti-EGFR, anti-IGF, anti-VEGF, anti-TNFalpha, anti-IL2 and anti-IgE antibodies.

The term “antibody”, “antibodies”, or “immunoglobulin(s)” is used herein in the broadest sense and encompasses various antibody structures, including but not limited to monoclonal antibodies, polyclonal antibodies, monospecific antibodies, multispecific antibodies (e.g., bispecific antibodies), and antibody fragments so long as they exhibit the desired antigen-binding activity. There are various classes of immunoglobulins: IgA, IgD, IgE, IgG, IgM, IgY, IgW. Preferably the antibody is an IgG antibody, more preferably an IgG1 or an IgG4 antibody.

Antibodies can be of any species and include chimeric, humanized and human antibodies. “Chimeric” antibodies are molecules in which antibody domains or regions are derived from different species. For example, the variable region of heavy and light chain can be derived from rat or mouse antibody and the constant regions from a human antibody. In “humanized” antibodies only minimal sequences are derived from a non-human species. Often only the CDR amino acid residues of a human antibody are replaced with the CDR amino acid residues of a non-human species such as mouse, rat, rabbit or llama. Sometimes a few key framework amino acid residues with impact on antigen binding specificity and affinity are also replaced by non-human amino acid residues.

Typically, antibodies are tetrameric polypeptides composed of two pairs of a heterodimer each formed by a heavy and a light chain. Stabilization of both the heterodimers as well as the tetrameric polypeptide structure occurs via interchain disulfide bridges. Each chain is composed of structural domains called “immunoglobulin domains” or “immunoglobulin regions” whereby the terms “domain” or “region” are used interchangeably. Each domain contains about 70-110 amino acids and forms a compact three-dimensional structure. Both heavy and light chain contain at their N-terminal end a “variable domain” or “variable region” with less conserved sequences which is responsible for antigen recognition and binding. The variable region of the light chain is also referred to as “VL” and the variable region of the heavy chain as “VH”.

An “antibody fragment” or “antigen-binding fragments” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′) 2; diabodies; linear antibodies; single-chain antibody molecules (e.g. scFv); and multispecific antibodies formed from antibody fragments. Fab fragments consist of the variable regions of both chains, which are held together by the adjacent constant region. These may be formed by protease digestion, e.g., with papain, from conventional antibodies, but similarly Fab fragments may also be produced by genetic engineering. Further antibody fragments include F(ab′)2 fragments, which may be prepared by proteolytic cleavage with pepsin.

Using genetic engineering methods it is possible to produce shortened antibody fragments which consist only of the variable regions of the heavy (VH) and of the light chain (VL). These are referred to as Fv fragments (Fragment variable=fragment of the variable part). Since these Fv-fragments lack the covalent bonding of the two chains by the cysteines of the constant chains, the Fv fragments are often stabilized. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins are known to the person skilled in the art. Thus, antibody fragments and antigen-binding fragments further include Fv-fragments and particularly scFv.

In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerisation of the scFv, scFv were prepared as fusion proteins with multimerisation domains. The multimerisation domains may be, e.g. the CH3 region of an IgG or coiled coil structure (helix structures) such as Leucine-zipper domains. However, there are also strategies in which the interaction between the VH/VL regions of the scFv is used for the multimerisation (e.g. dia-, tri- and pentabodies). By diabody the skilled person means a bivalent homodimeric scFv derivative. The shortening of the linker in a scFv molecule to 5-10 amino acids leads to the formation of homodimers in which an inter-chain VH/VL-superimposition takes place. Diabodies may additionally be stabilized by the incorporation of disulfide bridges. Examples of diabody-antibody proteins are known from the prior art.

By minibody the skilled person means a bivalent, homodimeric scFv derivative. It consists of a fusion protein which contains the CH3 region of an immunoglobulin, preferably IgG, most preferably IgG1 as the dimerisation region which is connected to the scFv via a Hinge region (e.g. also from IgG1) and a linker region. Examples of minibody-antibody proteins are known from the prior art.

By triabody the skilled person means a: trivalent homotrimeric scFv derivative. ScFv derivatives wherein VH-VL is fused directly without a linker sequence lead to the formation of trimers.

The skilled person will also be familiar with so-called miniantibodies which have a bi-, tri- or tetravalent structure and are derived from scFv. The multimerisation is carried out by di-, tri- or tetrameric coiled coil structures. In a preferred embodiment of the present invention, the gene of interest is encoded for any of those desired polypeptides mentioned above, preferably for a monoclonal antibody, a derivative or fragment thereof.

Further encompassed is a single-domain antibody (sdAb), also be referred to as nanobody, which is an antibody fragment of a single monomeric variable antibody domain. Single-domain antibodies are typically engineered from heavy chain antibodies found in camelids (VHH fragments) or cartilaginous fishes (VNAR fragments).

The immunoglobulin fragments composed of the CH2 and CH3 domains of the antibody heavy chain are called “Fc fragments”, “Fc region” or “Fc” because of their crystallization propensity (Fc=fragment crystallizable). These may be formed by protease digestion, e.g. with papain or pepsin from conventional antibodies but may also be produced by genetic engineering. The N-terminal part of the Fc fragment might vary depending on how many amino acids of the hinge region are still present.

Antibodies comprising an antigen-binding fragment and an Fc region may also be referred to as full-length antibody. Full-length antibody may be mono-specific and multispecific antibodies. Multispecific antibodies are antibodies which have at least two different antigen-binding sites each of which bind to different epitopes. A multispecific antibody includes bispecific and trispecific antibodies. A bispecific antibody has two different binding binding sites. Multispecific antibodies also include antibody formats other than full-length antibodies such as antibody-derived molecules.

Bispecific antibodies typically combine antigen-binding specificities for target cells (e.g., malignant B cells) and effector cells (e.g., T cells, NK cells or macrophages) in one molecule. Exemplary bispecific antibodies, without being limited thereto are diabodies, BiTE (Bi-specific T-cell Engager) formats and DART (Dual-Affinity Re-Targeting) formats. The diabody format separates cognate variable domains of heavy and light chains of the two antigen binding specificities on two separate polypeptide chains, with the two polypeptide chains being associated non-covalently. The DART format is based on the diabody format, but it provides additional stabilization through a C-terminal disulfide bridge. Trispecific antibodies are monoclonal antibodies which combine three antigen-binding specificities. They may be build on bispecific-antibody technology that reconfigures the antigen-recognition domain of two different antibodies into one bispecific molecule. For example, trispecific antibodies have been generated that target CD38 on cancer cells and CD3 and CD28 on T cells. Multispecific antibodies are particularly difficult to product with high product quality.

The term “antibody-derived molecule” as used herein refers to any molecule comprising at least an antigen-binding moiety that is structurally related to antibodies. It includes modified full-length mono- or bispecific antibodies further modified with an additional antigen binding moiety or smaller antibody formats including the ones described herein.

The term “antibody mimetic” as used herein refers to proteins that bind to specific antigens in a manner similar to antibodies, but that are not structurally related to antibodies. Antibody mimetic include, without being limited thereto an anticalin, an affibody, an adnectin, a monobody, a DARPin, an affimer, and an affitin.

A single-domain antibody (sdAb) may also be referred to as nanobody. The person skilled in the art will understand that the protein may comprise more than one antigen-binding domain and hence may be multivalent, preferably bivalent (e.g., a bivalent sdAb or a bivalent anticalin or any other bivalent antibody mimetic).

Another preferred therapeutic protein is a fusion protein, such as an Fc-fusion protein. Thus, the invention can be advantageously used for production of fusion proteins, such as Fc-fusion proteins. The effector part of the fusion protein can be the complete sequence or any part of the sequence of a natural or modified heterologous protein. The immunoglobulin constant domain sequences may be obtained from any immunoglobulin subtypes, such as IgG1, IgG2, IgG3, IgG4, IgA1 or IgA2 subtypes or classes such as IgA, IgE, IgD or IgM. Preferentially they are derived from human immunoglobulin, more preferred from human IgG and even more preferred from human IgG1 and IgG2. Non-limiting examples of Fc-fusion proteins are MCP1-Fc, ICAM-Fc, EPO-Fc and scFv fragments or the like coupled to the CH2 domain of the heavy chain immunoglobulin constant region comprising the N-linked glycosylation site. Fc-fusion proteins can be constructed by genetic engineering approaches by introducing the CH2 domain of the heavy chain immunoglobulin constant region comprising the N-linked glycosylation site into another expression construct comprising for example other immunoglobulin domains, enzymatically active protein portions, or effector domains. Thus, an Fc-fusion protein according to the present invention comprises also a single chain Fv fragment linked to the CH2 domain of the heavy chain immunoglobulin constant region comprising, e.g., the N-linked glycosylation site.

The term “cytokine” refers to small proteins, which are released by cells and act as intercellular mediators, for example influencing the behavior of the cells surrounding the secreting cell. Cytokines may be secreted by immune cells or other cells, such as T-cells, B-cells, NK cells and macrophages. Cytokines may be involved in intercellular signaling events, such as autocrine signaling, paracrine signaling and endocrine signaling. They may mediate a range of biological processes including, but not limited to immunity, inflammation, and hematopoiesis. Cytokines may be chemokines, interferons, interleukins, lymphokines or tumor necrosis factors.

As used herein, “growth factor” refers to proteins or polypeptides that are capable of stimulating cell growth.

The heterologous polynucleotide flanked by the two transposon flanking regions may further comprises a sequence encoding a selectable marker. As used herein, the terms “selectable marker” and “selection marker” are used synonymously herein and refer to a gene introduced into a cell that allows for selection in cell culture or a single cell clone. A selectable marker may be an antibiotic resistance gene, such as an ampicillin, a chloramphenicol, a tetracycline, a kanamycin resistance gene, pyromycin acetyltransferase, blasticidin acetyltransferase enzyme, hygromycin B phosphotransferase enzyme, or aminoglycoside 3′ phosphotransferase enzyme. A selectable marker may be also a gene encoding a fluorescent marker, such as green fluorescent protein (GFP), eGFP, monomeric red fluorescent proteins (mRFP) (e.g., mCherry), phycoerythrin or a luminescent marker, such as luciferase. A selectable marker may be also a split selectable marker. A split selectable marker comprises split marker segments fused to inteins, i.e., protein splicing elements, and expressed on separate vectors, which are re-joined via protein trans-splicing to reconstitute a full-length marker protein in host cells receiving the intended vectors as described e.g. by N. Jillette et al., (Nature Communications, 2019, 10:4968). Further suitable selectable marker are metabolic selectable marker, such as a selectable marker gene encoding glutamine synthetase (GS) or dihydrofolate reductase (DHFR), preferably GS.

The DNA transposon of the present invention can be located on any DNA molecule. Particularly, the DNA transposon may be present on a vector, particularly an expression vector, such as a plasmid DNA, a plasmid free of antibiotic resistance markers (pFAR), a minicircle (MC), a doggybone DNA (dbDNA), a Bacterial Artificial Chromosome (BAC), a Yeast Artificial Chromosome (YAC) or a non-integrative viral vector. In a preferred embodiment, the DNA transposon is present on a plasmid DNA or a BAC.

In another aspect, the invention relates to an expression vector comprising the DNA transposon according to the invention.

In yet another aspect, a eukaryotic cell is provided comprising the DNA transposon according to the invention. The eukaryotic cell may be any eukaryotic cell, such as a yeast, insect or mammalian cell. In case of an insect cell, the insect cell is not an insect cell of the family aphididae or the suborder Sternorrhyncha. Preferably the eukaryotic cell is a yeast or a mammalian cell, more preferably a mammalian cell, even more preferably a rodent or human cell, such as Chinese hamster ovary (CHO) cell or HEK293 cell derived cells. The cells are cells maintained in culture, such as cell lines or cell line derived cells, i.e., immortalized cells, or primary cells ex vivo. Suitable eukaryotic cells are further exemplified with regard to the methods of the invention and the same applies with regard to the eukaryotic cell of the invention.

In certain embodiment, the mammalian cell may comprise one or more viral gene as heterologous polynucleotide flanked by the transposon flanking regions, such as a viral gene from AAV, preferably an AAV rep and/or cap gene and optionally a helper virus gene, such as from HSV or AdV. Such a mammalian cell may be suitable as viral packaging cell. Thus, also provided is the use of a mammalian cells wherein the heterologous gene is one or more viral gene(s) for virus production, preferably AAV production.

In yet another aspect, a non-human transgenic animal is provided, wherein the transgenic animal comprises the DNA transposon according to the invention. The non-human transgenic animal is preferably a mammal, such as a rodent, a ruminant or a pig.

### A Recombinant Transposase

The present invention further relates in one aspect to a recombinant transposase comprising at least one heterologous nuclear localization signal (NLS) fused to the transposase, wherein the transposase is a Acyrthosiphon pisum transposase. The at least one heterologous NLS may be fused to the N-terminus, the C-terminus or to an internal region. The person skilled in the art would understand that the heterologous NLS is to be introduced without interfering with the transposase activity. Thus, in a preferred embodiment the recombinant transposase comprises at least a heterologous C-terminal and/or N-terminal NLS, i.e., at least a C-terminal or an N-terminal or a C-terminal and N-terminal heterologous NLS. In a more preferred embodiment, the recombinant transposase comprises at least a heterologous C-terminal and N-terminal NLS. Preferably the recombinant transposase comprises an amino acid sequence having at least 90% sequence identity with amino acids 10 to 585 of SEQ ID NO: 4 or 26, preferably with amino acids 10 to 585 of SEQ ID NO: 4. More preferably the recombinant transposase comprises an amino acid sequence having at least at least 95%, at least 97%, at least 98% or at least 99% sequence identity with amino acids 10 to 585 of SEQ ID NO: 4 or 26, preferably SEQ ID NO: 4, or having at least 90%, at least 95%, at least 97%, at least 98% or at least 99% sequence identity with SEQ ID NO: 4 or 26, preferably SEQ ID NO: 4. In certain embodiments the recombinant transposase has the about same or better transposase activity compared to the transposase having the amino acid sequence of SEQ ID NO: 4, wherein the transposase activity is preferably determined as accelerated recovery from selection and/or productivity using a DNA transposon comprising a transposon flanking region having the nucleotide sequence of SEQ ID NO: 2 and 3 or the nucleotide sequence of SEQ ID NO: 29 and 30 and encoding a heavy and a light chain of an antibody as protein of interest. Determination of accelerated recovery from selection and/or productivity using a DNA transposon comprising a transposon flanking region having the nucleotide sequence of SEQ ID NO: 2 and 3 or the nucleotide sequence of SEQ ID NO: 29 and 30 and encoding a heavy and a light chain of an antibody as protein of interest is, e.g., exemplified in Example 9. The about same or better transposase activity means at least 90%, preferably at least 95%, more preferably at least 98% and even more preferably at least 100% compared to the transposase having the amino acid sequence of SEQ ID NO: 4. In certain embodiments, the recombinant transposase according to the invention comprises an amino acid sequence of amino acids 10 to 585 of SEQ ID NO: 4 or SEQ ID NO: 26, preferably amino acids 10 to 585 of SEQ ID NO: 4. In a preferred embodiments, the recombinant transposase according to the invention comprises the amino acid sequence of SEQ ID NO: 4 or SEQ ID NO: 26, preferably SEQ ID NO: 4.

In a related aspect, the present invention relates to a recombinant transposase comprising an amino acid sequence having at least 90% sequence identity with amino acids 10 to 585 of SEQ ID NO: 4. The recombinant transposase according to the invention may be an Acyrthosiphon pisum transposase, but may also originate from a different species, such as Aphis craccivora. The Aphis craccivora transposase (SEQ ID NO: 26) has a sequence identity of 98.3% with the amino acid sequence of SEQ ID NO: 4. Thus, in certain embodiments, the recombinant transposase according to the invention is an Acyrthosiphon pisum transposase or an Aphis craccivora transposase. Preferably at least one heterologous NLS is fused to the transposase. The at least one heterologous NLS may be fused to the N-terminus, the C-terminus or to an internal region. The person skilled in the art would understand that the heterologous NLS is to be introduced without interfering with the transposase activity. Thus, in a preferred embodiment the recombinant transposase comprises at least a heterologous C-terminal and/or N-terminal NLS, i.e., at least a C-terminal or an N-terminal or a C-terminal and N-terminal heterologous NLS. In a more preferred embodiment, the recombinant transposase comprises at least a heterologous C-terminal and N-terminal NLS. Preferably, the recombinant transposase comprises an amino acid sequence having at least 90%, at least 95%, at least 97%, at least 98% or at least 99% sequence identity with amino acids 10 to 585 of SEQ ID NO: 4, or having at least 90%, at least 95%, at least 97%, at least 98% or at least 99% sequence identity with SEQ ID NO: 4. In certain embodiments the recombinant transposase comprises an amino acid sequence having at least 95%, preferably at least 97%, at least 98% or at least 99% sequence identity with amino acids 10 to 585 of SEQ ID NO: 26, or having at least 95%, preferably at least 97%, at least 98% or at least 99% sequence identity with SEQ ID NO: 26. In certain embodiments the recombinant transposase has the about same or better transposase activity compared to the transposase having the amino acid sequence of SEQ ID NO: 4, wherein the transposase activity is preferably determined as accelerated recovery from selection and/or productivity using a DNA transposon comprising a transposon flanking region having the nucleotide sequence of SEQ ID NO: 2 and 3 or SEQ ID NO: 29 and 30 and encoding a heavy and a light chain of an antibody as protein of interest. Determination of selection stringency and/or productivity using a DNA transposon comprising a transposon flanking region having the nucleotide sequence of SEQ ID NO: 2 and 3 or SEQ ID NO: 29 and 30 and encoding a heavy and a light chain of an antibody as protein of interest is exemplified in Example 9. The same or better transposase activity means at least 90%, preferably at least 95% more preferably at least 98% and even more preferably at least 100% compared to the transposase having the amino acid sequence of SEQ ID NO: 4. In certain embodiments, the recombinant transposase according to the invention comprises an amino acid sequence of amino acids 10 to 585 of SEQ ID NO: 4 or SEQ ID NO: 26, preferably SEQ ID NO: 4, more preferably the amino acid sequence of SEQ ID NO: 4. In certain embodiments, if the transposase comprises an amino acid sequence of amino acids 10 to 585 of SEQ ID NO: 26 or an amino acid sequence having at least 99% sequence identity with amino acids 10 to 585 of SEQ ID NO: 26, the DNA transposon to be used in combination is preferably a DNA transposon wherein (i) the one transposon flanking region comprises a sequence having at least 90% (preferably at least 95%, at least 97%, at least 98% at least 99% or 100%) sequence identity with at least SEQ ID NO: 64, preferably with at least SEQ ID NO; 63, at least SEQ ID NO: 62 or SEQ ID NO: 29; and/or the other transposon flanking region a sequence having at least 97% (preferably at least 98%, at least 99% or 100%) sequence identity with at least SEQ ID NO: 60 or 61, preferably with at least SEQ ID NO: 67, at least SEQ ID NO: 66, at least SEQ ID NO: 65 or more preferably with SEQ ID NO: 30, or (ii) wherein the one and the other transposon flanking regions are inverted and comprise reverse complementary sequences of the sequences recited in (i).

In certain preferred embodiments (i) the one transposon flanking region comprises a sequence having at least 95% (preferably at least 97%, at least 98%, at least 99% or at least 100%) sequence identity with SEQ ID NO: 29, 62 or 63 and/or the other transposon flanking region comprises a sequence having at least 97% (preferably at least 98%, at least 99% or at least 100%) sequence identity with SEQ ID NO: 30, 65 or 66, preferably SEQ ID NO: 30; or (ii) wherein the one and the other transposon flanking regions are inverted and comprise reverse complementary sequences of the sequences recited in (i) and the recombinant transposase is any transposase according to the invention. In other preferred embodiments, the DNA transposon is any DNA transposon according to the invention (such as AP or AC derived) and the recombinant transposase comprising an amino acid sequence having at least 99% sequence identity with amino acids 10 to 585 of SEQ ID NO: 4, optionally further comprising at least one mutation, wherein the at least one mutation is an amino acid substitution selected from the group consisting of K87Y, Q273V, V212I/I215L, I363V/K365S, K87Y/Q273V, K87Y/A264S/Q273V, A264S/Q273V, S270P/Q273V, K87Y/A264S, L583F, K576I, S372E, S277N and any combination thereof, and/or the at least one mutation is a deletion of N584 and/or E585, wherein the indicated amino acid position of the substitution and/or deletion corresponds to the amino acid position in the sequence of SEQ ID NO: 4. Preferably, the recombinant transposase comprises an amino acid sequence having at least 99% sequence identity with amino acids 10 to 585 of SEQ ID NO: 4 or further comprises at least one mutation, wherein the at least one mutation is K87Y or a combination of Q273V, V212/I215L and I363V/K365S.

The recombinant transposase according to the invention is able to transpose a transposon comprising a heterologous polynucleotide flanked by transposon flanking regions, wherein the left transposon flanking region has the nucleotide sequence of SEQ ID NO: 2 and the right transposon flanking region has the nucleotide sequence of SEQ ID NO: 3, or the left transposon flanking region has the nucleotide sequence of SEQ ID NO: 29 and the right transposon flanking region has the nucleotide sequence of SEQ ID NO: 30.

The heterologous NLS is preferably fused to the C-terminus and/or the N-terminus of the recombinant transposase of the invention. The heterologous NLS may be any NLS that mediates the transport of a protein from the cytoplasm into the nucleus. Preferably the NLS that mediates the transport of a protein from the cytoplasm into the nucleus in a mammalian cell. Typically, NLS are derived from a eukaryotic protein, preferably a mammalian protein, or a viral protein. The heterologous NLS may be classical nuclear localization signals (cNLS), including a monopartite NLS (MP NLS), such as the sequence of SEQ ID NO: 5 and bipartite NLS (BP NLS) such as the amino acid sequence of SEQ ID NO: 14, a non-classical NLS (ncNLS) any other type of NLS (also referred to as special NLS). The heterologous NLS (e.g., SEQ ID NO: 5) may be directly fused to the recombinant transposase or via a peptide linker (e.g., SEQ ID NO: 22, wherein the linker has the sequence of SEQ ID NO: 52).

The recombinant transposase according to the invention may further comprise at least one mutation. Thus, in certain embodiments the recombinant transposase comprises at least one mutation and is hyperactive compared to the transposase of SEQ ID NO: 4. Hyperactivity can be driven by a variety of reasons, such as half-life, stability, folding, activity, DNA binding and dimerization. Exemplary mutations, without being limited thereto are amino acid substitutions selected from the group consisting of K87Y, Q273V, V212I/I215L, I363V/K365S, K87Y/Q273V, K87Y/A264S/Q273V, A264S/Q273V, S270P/Q273V, K87Y/A264S, L583F, K576I, S372E, S277N and any combination thereof, and/or a deletion of N584 and/or E585, wherein the indicated amino acid position of the substitution and/or deletion corresponds to the amino acid position in the sequence of SEQ ID NO: 4. Preferably, the at least one mutation is an amino acid substitution selected from the group consisting of K87Y, Q273V, V212/I215L, I363V/K365S, A264S/Q273V, K87Y/A264S, L583F, or a combination thereof, and/or the at least one mutation is a deletion of N584 and/or E585. In certain embodiments, the at least one mutation is 1 to 7 amino acid substitutions, preferably 1 to 5 amino acid substitutions and/or a deletion of N584 and/or E585. In a more specific embodiment, the at least one mutation is 1 to 7 mutations, preferably 1 to 5 mutations. More preferably, the at least one mutation is the substitution K87Y or a combination of Q273V, V212/I215L and I363V/K365S. In certain specific embodiments the recombinant transposase comprises an amino acid sequence having at least 99% sequence identity with amino acids 10 to 585 of SEQ ID NO: 4 and further at least one mutation, wherein the one mutation is 1 to 5 amino acid substitutions selected from the group consisting of K87Y, Q273V, V212/I215L, I363V/K365S, A264S/Q273V, K87Y/A264S, L583F and optionally a deletion of N584 and/or E585, preferably wherein the at least one mutation is the substitution K87Y or a combination of Q273V, V212/I215L and I363V/K365S. The transposase activity is preferably determined as accelerated recovery from selection and/or productivity using a DNA transposon comprising a transposon flanking region having the nucleotide sequence of SEQ ID NO: 2 and 3 or the nucleotide sequence of SEQ ID NO: 29 and 30 and encoding a heavy and a light chain of an antibody as protein of interest (e.g., as exemplified in Example 9). A hyperactive transposase means more than 100%, preferably more than about 120%, more preferably more than about 150%, even more preferably more than about 200% transposase activity compared to a transposase having the amino acid sequence of SEQ ID NO: 4. In a preferred embodiment the recombinant transposase comprising the at least one mutation according to the invention comprises an amino acid sequence having at least 90%, at least at least 95%, at least 97%, at least 98% or at least 99% sequence identity with amino acids 10 to 585 of SEQ ID NO: 4 or 26, preferably with SEQ ID NO: 4 or 26, more preferably SEQ ID NO: 4.

In yet another aspect, the invention relates to a polynucleotide encoding a transposase comprising an amino acid sequence having at least 90% sequence identity with at least amino acids 10 to 585 of SEQ ID NO: 4 or SEQ ID NO: 26, operably linked to a eukaryotic promoter. In certain embodiments, the eukaryotic promoter is not the natural transposase promoter, i.e., a heterologous eukaryotic promoter. The promoter may be any promoter compatible with or active in the eukaryotic host cell. Suitable eukaryotic promoter are, without being limited thereto an EF1a promoter, a cytomegalovirus (CMV) promoter, a GAPDH promoter, a CAG promoter, a Herpes Simplex Virus thymidine kinase (HSV-TK) promoter, a Murine Stem Cell Virus (MSCV) promoter, a spleen focus-forming virus (SFFV) promoter, an SV40 promoter and an actin promoter, a PGK promoter and an ubiquitin promoter. Further, the promoter can be an inducible promoter. The term “operably linked” is uses synonymously with “under the control of” means that an expression control sequence, such as a promoter (or a transcription termination sequence), functionally controls the expression of one or more gene(s) of interest or the like. Typically, the expression control sequence, such as the promoter (or a transcription termination sequence) is contiguous with the gene(s) of interest.

In yet another aspect, the invention relates to a polynucleotide encoding the recombinant transposase according to the invention. The polynucleotide may be a DNA or an RNA. The transposase may be introduced into a host cell as protein, e.g., by microinjection, or as DNA or mRNA by transduction or transfection. A DNA polynucleotide encoding the recombinant transposase may be part of an expression vector, such as a plasmid DNA, a plasmid free of antibiotic resistance markers (pFAR), a minicircle (MC), a doggybone DNA (dbDNA), a Bacterial Artificial Chromosome (BAC), a Yeast Artificial Chromosome (YAC) or a viral vector, preferably a non-integrative viral vector. Thus, the source of the recombinant transposase may be a protein, an expression vector or an mRNA. In a preferred embodiment, the recombinant transposase is introduced into the host cell by transfection of an expression vector, such as a plasmid DNA, or an mRNA. In a preferred embodiment one or more codons of the polynucleotide encoding the transposase are selected for eukaryotic cell expression, preferably for yeast, insect or mammalian cell expression, more preferably for mammalian cell expression, such as rodent or human cell expression. In other words, the polynucleotide encoding the transposase is codon-optimized, particularly codon-optimized for the expression host cell, such as a mammalian cell.

In yet another aspect of the invention an expression vector is provided encoding the recombinant transposase according to the invention or comprising the polynucleotide encoding the recombinant transposase according to the invention. In certain embodiments, the expression vector is a plasmid DNA, a plasmid free of antibiotic resistance markers (pFAR), a minicircle (MC), a doggybone DNA (dbDNA), a Bacterial Artificial Chromosome (BAC), a Yeast Artificial Chromosome (YAC) or a viral vector, preferably a plasmid DNA or a BAC.

In yet another aspect of the invention an isolated mRNA is provided encoding the recombinant transposase according to the invention. The mRNA may be modified for more stability. The person skilled in the art would know mRNA modifications optimized for translation and stabilization, such as nucleotide substitution, e.g., using pseudo-uridine, 5-methyl-cytosine or 5-methoxy-uridine and/or mRNA capping for translation, such as an mCAP analog (m7G(5′)ppp(5′)G) or an ARCA cap (Anti Reverse Cap Analog, 3′-O-Me-m7G(5′)ppp(5′)G).

In yet another aspect of the invention an expression system or a kit is provided comprising (a) a recombinant transposase source selected from the group consisting of (i) the expression vector encoding the recombinant transposase according to the invention or comprising the polynucleotide encoding the recombinant transposase according to the invention; (ii) the isolated mRNA encoding the recombinant transposase according to the invention; and (iii) the recombinant transposase according to the invention; and (b) a DNA transposon according to the invention or an expression vector comprising the DNA transposon according to the invention. The expression system or kit may further comprise (c) a transfection agent and/or (d) a eukaryotic cell.

Uses of the DNA Transposon and/or the Recombinant Transposase

The present invention further relates to uses or methods of using the transposase/DNA transposon pair according to the invention, i.e., the DNA transposon according to the invention and the recombinant transposase according to the invention.

In one aspect, a pharmaceutical composition is provided comprising (a) the DNA transposon according to the invention; and (i) the recombinant transposase according to the invention or (ii) the isolated mRNA according to the invention; or (b) a human cell comprising the DNA transposon according to the invention, wherein the cell is preferably a patient autologous cell.

In another aspect, the pharmaceutical composition according to the invention is for use in gene therapy, or for use in treating cancer or treating an autoimmune disease. In certain embodiments the gene therapy may be a substitution gene therapy for treating a genetic disease, such as in a recessive genetic disease. In certain embodiments the cancer is a blood cancer or a solid cancer, preferably a blood cancer. Preferably the gene of interest encodes a chimeric antigen receptor (CAR) and the human cell is a human T cell or a human NK cell. In a preferred embodiment the use comprises ex vivo transfection of autologous cells of a patient followed by adoptive cell transfer of said autologous cell. Thus, in a specific embodiment the pharmaceutical composition is for use in treating cancer, particularly blood cancer, the autologous cell is a T cell or an NK cell and the treatment comprises ex vivo transfection of the autologous T cell or NK cell with the DNA transposon comprising a polynucleotide comprising a sequence encoding a CAR and subsequent adoptive cell transfer of the autologous T cell or NK cell comprising the DNA transposon to the patient.

The invention further relates to methods, particularly in vitro methods, for stably integrating a heterologous polynucleotide into a cell, such as when preparing a cell line, or for the production of a product of interest, particularly a protein of interest, or for the generation of a virus packaging cell line.

In one aspect, the invention relates to a method for preparing a cell comprising a stably integrated heterologous polynucleotide, comprising (a) introducing a DNA molecule comprising the DNA transposon according to the invention or the expression vector comprising said DNA transposon according to the invention into a eukaryotic cell, wherein the heterologous polynucleotide comprises a sequence encoding a gene of interest, a complementary DNA (cDNA), a genome of interest or another genetic element, and wherein the heterologous polynucleotide further comprises a sequence encoding a selectable marker; (b) introducing a recombinant transposase source into said eukaryotic cell, wherein the recombinant transposase source is selected from the group consisting of: (i) the expression vector encoding the recombinant transposase of the invention, (ii) the isolated mRNA of the invention, and (iii) the recombinant transposase of the invention; and (c) culturing the eukaryotic cell in a medium under conditions to select for the selectable marker, wherein the DNA transposon is stably integrated into the genome of the eukaryotic cell. The method may further comprise a step (d) isolating a single clone for clonal expansion to prepare a monoclonal cell line.

In another aspect, the invention relates to a method for preparing a protein of interest, comprising (a) introducing a DNA molecule comprising the DNA transposon according to the invention or the expression vector comprising said DNA transposon according to the invention into a eukaryotic cell, wherein the heterologous polynucleotide comprises a sequence encoding at least one protein of interest and further a sequence encoding a selectable marker; (b) introducing a recombinant transposase source into said eukaryotic cell, wherein the recombinant transposase source is selected from the group consisting of: (i) an expression vector encoding the recombinant transposase according to the invention, (ii) the isolated mRNA according to the invention; and (ii) the recombinant transposase according to the invention; (b) culturing the eukaryotic cell in a medium under conditions to select for the selectable marker, wherein the heterologous polynucleotide comprising a sequence encoding the at least one protein of interest and a sequence encoding a selectable marker is stably integrated into the genome of the eukaryotic cell (c) optionally isolating a single clone for clonal expansion to prepare a monoclonal cell line; (d) culturing the eukaryotic cell under conditions to produce the protein of interest; and (d) harvesting and optionally purifying the protein of interest.

In yet another aspect, the invention relates to a method for preparing a virus or virus like particle of interest or to a method for preparing a virus packaging cell line as specified herein in item 39 or 40, respectively.

Generally, any DNA transposon according to the invention can be used in combination with any recombinant transposase according to the invention. However, the person skilled in the art will understand that each DNA transposon needs to be tested in combination with a certain recombinant transposase, as e.g., shown in the Examples. The identified recombinant transposases derived from AP (SEQ ID NO: 4) and AC (SEQ ID NO: 26) efficiently transpose a DNA transposon comprising AP (SEQ ID NOs: 2 and 3) and AC (SEQ ID NOs: 29 and 30) transposon flanking regions, respectively, particularly resulting in a comparable selection behavior and productivity when using and/or detecting the same heterologous protein expression. It was surprisingly found that the recombinant AP transposase even more efficiently transposes a DNA transposon comprising AC (SEQ ID NOs: 29 and 30) transposon flanking regions. The same applies to the truncated forms of AP (SEQ ID NOs: 2 and/or 3) or AC transposon flanking regions (SEQ ID NOs: 29 and/or 30) as described herein.

In certain preferred embodiments of the uses and methods according to the invention (i) a DNA transposon comprising AC derived transposon flanking regions and an AC or AP transposase, or (ii) a DNA transposon comprising AP derived transposon flanking regions and an AP transposase is used. In a particularly preferred embodiment, a DNA transposon comprising AC derived transposon flanking regions and an AP transposase is used.

For example, in certain embodiments the DNA molecule in step (a) comprising the DNA transposon comprising a heterologous polynucleotide flanked by transposon flanking regions or the expression vector encoding said DNA transposon, wherein (i) the one transposon flanking region comprises a sequence having at least 95% sequence identity with SEQ ID NO: 29, 62, 63 or 64 and/or the other transposon flanking region comprises a sequence having at least 97% sequence identity with SEQ ID NO: 30, 65, 66, 67, 60 or 61; or (ii) wherein the one and the other transposon flanking regions are inverted and comprise reverse complementary sequences of the sequences recited in (i) and the recombinant transposon source in step (b) encodes or is a recombinant transposase comprising an amino acid sequence having at least 90% sequence identity with amino acids 10 to 585 of SEQ ID NO: 4, optionally further comprising at least one mutation and/or at least one heterologous NLS fused to the transposase as defined for the recombinant transposase according to the invention.

Exemplary mutations, without being limited thereto are amino acid substitutions selected from the group consisting of K87Y, Q273V, V212I/I215L, I363V/K365S, K87Y/Q273V, K87Y/A264S/Q273V, A264S/Q273V, S270P/Q273V, K87Y/A264S, L583F, K576I, S372E, S277N and any combination thereof, and/or a deletion of N584 and/or E585, wherein the indicated amino acid position of the substitution and/or deletion corresponds to the amino acid position in the sequence of SEQ ID NO: 4. Preferably, the at least one mutation is an amino acid substitution selected from the group consisting of K87Y, Q273V, V212/I215L, I363V/K365S, A264S/Q273V, K87Y/A264S, L583F, or a combination thereof, and/or the at least one mutation is a deletion of N584 and/or E585. In certain embodiments, the at least one mutation is 1 to 7 amino acid substitutions, preferably 1 to 5 amino acid substitutions and/or a deletion of N584 and/or E585. In a more specific embodiment, the at least one mutation is 1 to 7 mutations, preferably 1 to 5 mutations. More preferably, the at least one mutation is the substitution K87Y or a combination of Q273V, V212/I215L and I363V/K365S. In certain specific embodiments the recombinant transposase comprises an amino acid sequence having at least 99% sequence identity with amino acids 10 to 585 of SEQ ID NO: 4 and further at least one mutation, wherein the one mutation is 1 to 5 amino acid substitutions selected from the group consisting of K87Y, Q273V, V212/I215L, I363V/K365S, A264S/Q273V, K87Y/A264S, L583F and optionally a deletion of N584 and/or E585, preferably wherein the at least one mutation is the substitution K87Y or a combination of Q273V, V212/I215L and I363V/K365S.

In a preferred embodiment, the DNA transposon comprises AC derived transposon flanking regions and is used together with an AP transposase. In certain embodiments, the transposase comprises an amino acid sequence of amino acids 10 to 585 of SEQ ID NO: 26 or an amino acid sequence having at least 99% sequence identity with amino acids 10 to 585 of SEQ ID NO: 26, optionally further comprising at least one mutation as described herein, the DNA transposon to be used in combination is preferably a DNA transposon wherein (i) the one transposon flanking region comprises a sequence having at least 90% (preferably at least 95%, at least 97%, at least 98% at least 99% or 100%) sequence identity with at least SEQ ID NO: 64, preferably with at least SEQ ID NO; 63, at least SEQ ID NO: 62 or SEQ ID NO: 29; and/or the other transposon flanking region a sequence having at least 97% (preferably at least 98%, at least 99% or 100%) sequence identity with at least SEQ ID NO: 60 or 61, preferably with at least SEQ ID NO: 67, at least SEQ ID NO: 66, at least SEQ ID NO: 65 or more preferably with SEQ ID NO: 30, or (ii) wherein the one and the other transposon flanking regions are inverted and comprise reverse complementary sequences of the sequences recited in (i). The same as disclosed for the uses and methods of the invention similarly applies to the expression system or the kit according to the invention.

The protein of interest is preferably a therapeutic protein, such as a therapeutic protein selected from the group consisting of a cytokine, a hormone, a fusion protein, an antibody, an antibody-derived molecule and an antibody mimetic. Such therapeutic proteins are described in more detail herein above.

In certain embodiments of the methods of the invention, the DNA molecule comprising the DNA transposon according to the invention or the expression vector encoding the transposase according to the invention may be introduced by transfection ortransduction, preferably by transfection. Similarly, the recombinant transposase according to the invention or the mRNA encoding the recombinant transposase according to the invention is preferably introduced by transfection.

The methods for preparing according to the invention are in vitro methods. Eukaryotic host cells encompass particularly yeast cells and mammalian cells and are preferably mammalian cells. Yeast cells can be, without being limited thereto Saccharomyces cerevisiae, Pichia pastoris, Klyveromyces lactis or marxianus. Mammalian cells as used herein refer to all cells or cell lines of mammalian origin, such as human or rodent cells and may also be referred to as “host cell” or “mammalian host cell”. The cells as referred to herein are cells maintained in culture, such as cell lines or cell line derived cells, i.e., immortalized cells, or primary cells ex vivo. Primary cells are cells isolated from organ tissue or an organism and maintained in vitro for growth and/or adoptive cell transfer into a patient or subject. For adoptive cell transfer, the primary cell is preferably a patient autologous cell. The mammalian cells further comprise mammalian cell lines suitable for the production of a product of interest, such as a heterologous protein of interest and/or a non-coding RNA. The mammalian cells are preferably transformed and/or immortalized cell lines. Cell lines are adapted to serial passages in cell culture, preferably serum-free cell culture and/or preferably as suspension culture, and do not include primary non-transformed cells or cells that are part of an organ structure.

Preferably the mammalian host cell is a human or a rodent cell, more preferably a rodent cell, even more preferably a CHO cell. Preferred mammalian cells for heterologous protein production are rodent cells or human cells. Preferred examples of mammalian cells or mammalian cell lines are CHO cells (such as DG44 and K1), NSO cells, HEK293 cells (such as HEK293 cells and HEK293T cells) and BHK21 cells. Preferably the mammalian cells or mammalian cell lines are adapted to growth in suspension. In a preferred embodiment the mammalian cells or mammalian cell line is a CHO cell. In certain embodiments the mammalian cell is a HEK293 cell or a CHO cell or a HEK293 cell or a CHO cell derived cell, preferably the mammalian cell is a CHO cell or a CHO derived cell.

Suitable rodent cells may be e.g., hamster cells, particularly BHK21, BHK TK-, CHO, CHO-K1, CHO-DXB11 (also referred to as CHO-DUKX or DuxB11), a CHO—S cell and CHO-DG44 cells or the derivatives/progenies of any of such cell line. Particularly preferred are CHO cells, such as CHO-DG44, CHO-K1 and BHK21, and even more preferred are CHO-DG44 and CHO-K1 cells. Most preferred are CHO-DG44 cells. Glutamine synthetase (GS)-deficient derivatives of the mammalian cell, particularly of the CHO-DG44 and CHO-K1 cell are also encompassed. In one embodiment of the invention the mammalian cell is a Chinese hamster ovary (CHO) cell, preferably a CHO-DG44 cell, a CHO-K1 cell, a CHO DXB11 cell, a CHO—S cell, a CHO GS deficient cell or a derivative thereof. Suitable human cells are HEK293 or HEK293T cells. The host cells may also be murine cells such as murine myeloma cells, such as NSO and Sp2/0 cells or the derivatives/progenies of any of such cell line.

Preferably, CHO cells that allow for efficient cell line development processes are metabolically engineered, such as by endogenous glutamine synthetase (GS) knockout to facilitate selection with methionine sulfoximine (MSX). The term “GS gene knockout cell” as used herein refers to a cell in which the endogenous GS gene has been knocked out, i.e., deleted or disrupted, resulting in GS enzyme function disruption. Such cells may be referred to as GS−/− or GS−/+ cells, depending on whether both or only one allele has been deleted or disrupted, preferably GS−/− cells are used. Extracellular glutamine supplementation or a GS gene introduced by an expression vector is essential for cell survival of GS gene knockout cells. In a preferred embodiment the mammalian host cell is a CHO-K1 cell, more preferably a CHO-K1-GS (GS−/−) cell. Commonly used CHO cells for large-scale industrial production are often engineered to improve their characteristics in the production process, orto facilitate selection of recombinant cells. Such engineering includes, but is not limited to increasing apoptosis resistance, reducing autophagy, increasing cell proliferation, altered expression of cell-cycle regulating proteins, chaperone engineering, engineering of the unfolded protein response (UPR), engineering of secretion pathways and metabolic engineering.

Non-limiting examples of mammalian cells which can be used in the meaning of this invention are summarized in Table B. However, derivatives/progenies of those cells, other mammalian cells, including but not limited to human, mice, rat, monkey, and rodent cell lines, can also be used in the present invention, particularly for the production of biopharmaceutical proteins.

TABLE B
Exemplary mammalian production cell lines

| Cell line | Order Number |
|---|---|
| NS0 | ECACC No. 85110503 |
| Sp2/0-Ag14 | ATCC CRL-1581 |
| BHK21 | ATCC CCL-10 |
| BHK TK⁻ | ECACC No. 85011423 |
| Hak | ATCC CCL-15 |
| 2254-62.2 (BHK-21 derivative) | ATCC CRL-8544 |
| CHO | ECACC No. 8505302 |
| CHO wild type | ECACC 00102307 |
| CHO-K1 | ATCC CCL-61 |
| CHOZN ® | Merck SAFC |
| | GS−/− and DHFR−/− |
| CHO-DUKX | ATCC CRL-9096 |
| (=CHO duk⁻, CHO/dhfr⁻, CHO-DXB11) | |
| CHO-DUKX 5A-HS-MYC | ATCC CRL-9010 |
| CHO-DG44 | Urlaub G, et al., 1983. Cell. 33: 405-412. |
| CHO Pro-5 | ATCC CRL-1781 |
| CHO-S | Life Technologies A1136401; CHO-S is derived |
| | from CHO as disclosed in Deaven L L and Petersen |
| | D F, 1973, Chromosoma (Berl.) 41: 129-144; and |
| | Lewis N E et al., 2013, Nature Biotechnology 31: |
| | 759-765 |
| V79 | ATCC CCC-93 |
| B14AF28-G3 | ATCC CCL-14 |
| HEK 293 | ATCC CRL-1573 |
| COS-7 | ATCC CRL-1651 |
| U266 | ATCC TIB-196 |
| HuNS1 | ATCC CRL-8644 |
| CHL | ECACC No. 87111906 |
| CAP¹ | Wolfel J, et al., 2011. BMC Proc. 5(Suppl 8): P133. |
| PER.C6 ® | Pau et al., 2001. Vaccines. 19: 2716-2721. |
| H4-II-E | ATCC CRL-1548 |
| | ECACC No.87031301 |
| | Reuber, 1961. J. Natl. Cancer Inst. 26: 891-899. |
| | Pitot HC, et al., 1964. Natl. Cancer Inst. Monogr. |
| | 13: 229-245. |
| H4-II-E-C3 | ATCC CRL-1600 |
| H4TG | ATCC CRL-1578 |
| H4-II-E | DSM ACC3129 |
| H4-II-Es | DSM ACC3130 |

¹CAP (CEVEC's Amniocyte Production) cells are an immortalized cell line based on primary human amniocytes. They were generated by transfection of these primary cells with a vector containing the functions E1 and pIX of adenovirus 5. CAP cells allow for competitive stable production of recombinant proteins with excellent biologic activity and therapeutic efficacy as a result of authentic human posttranslational modification.

Cells are most preferred, when being established, adapted, and completely cultivated under serum free conditions, and optionally in media, which are free of any protein/peptide of animal origin. Commercially available media such as Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, CA), serum-free CHO Medium (Sigma), and protein-free CHO Medium (Sigma) are exemplary appropriate nutrient solutions. Any of the media may be supplemented as necessary with a variety of compounds, non-limiting examples of which are recombinant hormones and/or other recombinant growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics and trace elements. Any other necessary supplements may also be included at appropriate concentrations that would be known to those skilled in the art. For the growth and selection of genetically modified cells expressing a selectable gene a suitable selection agent is added to the culture medium.

The protein of interest encoded by the eukaryotic expression vector or produced by the methods of the invention is preferably produced in CHO cells in cell culture. Following expression, the recombinant protein is harvested and further purified. The antibody may be recovered from the culture medium as a secreted protein in the harvested cell culture fluid (HCCF) or from a cell lysate (i.e., the fluid containing the content of a cell lysed by any means, including without being limited thereto enzymatic, chemical, osmotic, mechanical and/or physical disruption of the cell membrane and optionally cell wall) and purified using techniques described herein. According to the invention the method comprises providing a harvested cell culture fluid comprising a protein of interest, such as an antibody as starting material, wherein the HCCF is from CHO cell culture. Preferably the protein of interest, such as the antibody, is recovered from the harvested cell culture fluid following cell separation, such as by filtration and/or centrifugation. Thus, in certain embodiments the harvest includes centrifugation, clarification and/or filtration to produce a harvested cell culture fluid, preferably followed by one or more filtration steps like ultrafiltration and/or diafiltration. The purified protein can optionally be formulated and optionally re-formulated into solid or liquid, preferably liquid compositions designed for the intended uses. Such formulations are in principle known to the person skilled in the art and can comprise e.g. buffers, stabilizers and/or other excipients for use as medical treatments of humans or animals.

In view of the above, it will be appreciated that the invention also encompasses the following items:

Item 1 provides a DNA transposon comprising a heterologous polynucleotide flanked by transposon flanking regions, wherein one transposon flanking region comprises the nucleotide sequence of SEQ ID NO: 38 the one transposon end; and the other transposon flanking region comprises the nucleotide sequence of SEQ ID NO: 39 at the other transposon end.

Item 2 further specifies the DNA transposon of item 1, wherein the one transposon flanking region comprises the nucleotide sequence of SEQ ID NO: 40 at one transposon end; and the other transposon flanking region comprises the nucleotide sequence of SEQ ID NO: 41 at the other transposon end.

Item 3 further specifies the DNA transposon of item 1 or 2, wherein the one and the other transposon flanking region each further comprise at least one inverted internal repeat (IIR) comprising (i) an internal repeat motif comprising the nucleotide sequence tggtctac and (b) its reverse complementary sequence (iii) separated by three to six, preferably four nucleotides, preferably the at least one inverted internal repeat (IIR) motif has the nucleotide sequence of SEQ ID NOs: 43.

Item 4 further specifies the DNA transposon of any one of items 1 to 3, wherein (a) the one transposon flanking region comprises the nucleotide sequence of SEQ ID NO: 45 or 46 and further an inverted internal repeat (IIR) motif having the nucleotide sequence of SEQ ID NO: 43 separated by about 50 to 200 nucleotides; and/or the other transposon flanking region comprises the nucleotide sequence of SEQ ID NO: 47, or a sequence having at least 95% sequence identity with SEQ ID NO: 47, wherein the sequence comprises at least the nucleotide sequence of SEQ ID NO: 41 and the nucleotide sequence of SEQ ID NO: 43; or (b) wherein the one and the other transposon flanking regions are inverted and comprise reverse complementary sequences of the sequences recited in (a); preferably wherein (a) the one transposon flanking region comprises the nucleotide sequence of SEQ ID NO: 45 or 46 and further an inverted internal repeat motif having the nucleotide sequence of SEQ ID NO: 43 separated by about 50 to 200 nucleotides and wherein the one transposon flanking region comprises a sequence having 85% sequence identity with SEQ ID NO: 10 or 63, or with SEQ ID NO: 2 or 29; and/or the other transposon flanking region comprises a sequence of SEQ ID NO: 47 or a sequence having at least 95% sequence identity with SEQ ID NO: 47, wherein the sequence comprises at least the nucleotide sequence of SEQ ID NO: 41 and the nucleotide sequence of SEQ ID NO: 43; or (b) wherein the one and the other transposon flanking regions are inverted and comprise reverse complementary sequences of the sequences recited in (i).

Item 5 further specifies the DNA transposon of any one of items 1 to 4, wherein (a) the one transposon flanking region comprises a sequence having at least 85% nucleotide sequence identity with SEQ ID NO: 2, 8, 10 or 58 and/or the other transposon flanking region comprises a sequence having at least 90% nucleotide sequence identity with SEQ ID NO: 3, 9, 11 or 59; or (b) wherein the one and the other transposon flanking regions are inverted comprising reverse complementary sequences of the sequences recited in (a). Item 6 further specifies the DNA transposon of any one of items 1 to 5, (1) wherein (a) the one transposon flanking region comprises the nucleotide sequence of SEQ ID NO: 2, 8, 10, or 58 or the nucleotide sequence of SEQ ID NO: 29, 62, 63 or 64 and/or the other transposon flanking region comprises the nucleotide sequence of SEQ ID NO: 3, 9, 11, 59, 48 or 49 or the nucleotide sequence of SEQ ID NO: 30, 65, 66, 67, 60 or 61; or (b) wherein the one and the other transposon flanking regions are inverted and comprise reverse complementary sequences of the sequences recited in (a); or (2) (a) the one transposon flanking region comprises a sequence having at least 95% sequence identity with SEQ ID NO: 29, 62, 63 or 64 and/or the other transposon flanking region comprises a sequence having at least 97% sequence identity with SEQ ID NO: 30, 65, 66, 67, 60 or 61; or (b) wherein the one and the other transposon flanking regions are inverted and comprise reverse complementary sequences of the sequences recited in (a).

Item 7 further specifies the DNA transposon of any one of the preceding items, wherein the heterologous polynucleotide comprises at least one sequence selected from the group consisting of a sequence encoding a gene of interest, a complementary DNA (cDNA), a genome of interest and another genetic element.

Item 8 further specifies the DNA transposon of item 7, wherein the heterologous polynucleotide comprises the at least one sequence under the control of a promoter and a transcription termination signal.

Item 9 further specifies the DNA transposon of item 8, wherein the promoter is a eukaryotic promoter, preferably (1) selected from the group consisting of an EF1a promoter, a CMV promoter, a GAPDH promoter, a CAG promoter, a Herpes Simplex Virus thymidine kinase (HSV-TK) promoter, a MSCV promoter, a SFFV promoter, an SV40 promoter and an actin promoter, a PGK promoter and an ubiquitin promoter, and/or (2) an inducible promoter.

Item 10 further specifies the DNA transposon of any one of the preceding items, wherein the heterologous polynucleotide comprises at least one sequence selected from the group consisting of a sequence or sequences encoding
(i) a heavy and/or a light chain of an antibody;
(ii) a secreted therapeutic recombinant protein;
(iii) a recombinant protein;
(iv) a transmembrane receptor;
(v) a non-coding RNA mediating RNA interference (RNAi), preferably selected from the group consisting of siRNA, shRNA, lncRNA and miRNA;
(vi) one or more viral proteins;
(vii) a viral genome or a viral genomic cDNA;
(viii) a ribozyme;
(ix) a binding motif;
(x) a regulatory DNA element,
(xi) another genetic element; and
(xii) a combination of any one of (i) to (xi).

Item 11 further specifies the DNA transposon of any one of the preceding items, wherein the heterologous polynucleotide further comprises a sequence encoding a selectable marker, preferably a metabolic selectable marker, more preferably glutamine synthetase (GS) or dihydrofolate reductase (DHFR), even more preferably GS.

Item 12 further specifies the DNA transposon of any one of the preceding items, wherein the transposon is transposable by a transposase comprising the amino acid sequence of SEQ ID NO: 4.

Item 13 further specifies the DNA transposon of any one of the preceding items, wherein the DNA transposon is present on a plasmid DNA, a plasmid free of antibiotic resistance markers (pFAR), a minicircle (MC), a doggybone DNA (dbDNA), a Bacterial Artificial Chromosome (BAC), a Yeast Artificial Chromosome or a non-integrative viral vector.

Item 14 provides an expression vector comprising the DNA transposon of any one of items 1 to 13.

Item 15 provides a recombinant transposase comprising at least one heterologous nuclear localization signal (NLS) fused to the transposase, preferably at least a heterologous C-terminal and/or N-terminal NLS, wherein the transposase is a Acyrthosiphon pisum transposase.

Item 16 further specifies the recombinant transposase of item 15, wherein the transposase comprises an amino acid sequence having at least 90%, preferably 98%, sequence identity with amino acids 10 to 585 of SEQ ID NO: 4.

Item 17 provides a recombinant transposase comprising an amino acid sequence having at least 90%, preferably 98%, sequence identity with amino acids 10 to 585 of SEQ ID NO: 4.

Item 18 further specifies the recombinant transposase of item 17, wherein (a) the transposase is a Acyrthosiphon pisum transposase or an Aphis craccivora transposase; and/or (b) the transposase comprises at least one heterologous NLS fused to the transposase.

Item 19 further specifies the recombinant transposase of any one of items 15 to 18, wherein the transposase comprises the amino acid sequence of amino acids 10 to 585 of SEQ ID NO: 4, preferably the amino acid sequence of SEQ ID NO: 4, or comprises the amino acid sequence of amino acids 10 to 585 of SEQ ID NO: 26, preferably the amino acid sequence of SEQ ID NO: 26.

Item 20 further specifies the recombinant transposase of any one of items 15 to 19, wherein the transposase comprises at least one mutation and is hyperactive compared to the transposase of SEQ ID NO: 4.

Item 21 further specifies the recombinant transposase of any one of items 15 to 20, wherein the transposase comprises at least one mutation, preferably wherein the at least one mutation is an amino acid substitution, preferably 1 to 7 or 1 to 5 amino acid substitutions, selected from the group consisting of K87Y, Q273V, V212I/I215L, I363V/K365S, K87Y/Q273V, K87Y/A264S/Q273V, A264S/Q273V, S270P/Q273V, K87Y/A264S, L583F, K576I, S372E, S277N and any combination thereof, and/or the at least one mutation is a deletion of N584 and/or E585, wherein the indicated amino acid position of the substitution and/or deletion corresponds to the amino acid position in the sequence of SEQ ID NO: 4, preferably the at least one mutation is an amino acid substitution selected from the group consisting of K87Y, Q273V, V212/I215L, I363V/K365S, A264S/Q273V, K87Y/A264S, L583F, or a combination thereof, and/or the at least one mutation is a deletion of N584 and/or E585, optionally wherein at least one heterologous NLS is fused to the transposase.

Item 22 provides a polynucleotide encoding a transposase comprising an amino acid sequence having at least 90%, preferably 98%, sequence identity with amino acids 10 to 585 of SEQ ID NO: 4 operably linked to a eukaryotic promoter.

Item 23 provides a polynucleotide encoding the recombinant transposase of any one of items 15 to 21.

Item 24 further specifies the polynucleotide of item 23, wherein the polynucleotide is a DNA or an RNA.

Item 25 further specifies the polynucleotide of any one of items 22 to 24, wherein one or more codons of the polynucleotide encoding the transposase are selected for eukaryotic cell expression, preferably for yeast or mammalian cell expression, more preferably mammalian cell expression (e.g., rodent or human cell expression).

Item 26 provides an expression vector comprising the polynucleotide of any one of items 22 to 25 or encoding the recombinant transposase of any one of items 15 to 21.

Item 27 provides an isolated mRNA encoding the recombinant transposase of any one of items 15 to 21.

Item 28 provides an expression system or a kit comprising (a) a recombinant transposase source selected from the group consisting of (i) the expression vector of item 26; (ii) the isolated mRNA of item 27; and (iii) the recombinant transposase of any one of items 15 to 21; and (b) a DNA transposon of any one of items 1 to 13 or an expression vector of item 14.

Item 29 provides a eukaryotic cell comprising the DNA transposon of any one of items 1 to 13.

Item 30 further specifies the eukaryotic cell of item 29, wherein the eukaryotic cell is a yeast or mammalian cell, preferably a mammalian cell, more preferably a rodent or human cell.

Item 31 further specifies the eukaryotic cell of item 29 as a mammalian cell and further that the heterologous polynucleotide flanked by the transposon flanking regions is one or more viral gene, preferably from AAV, more preferably an AAV rep and/or cap gene and optionally further at least one helper virus gene (e.g., from HSV or AdV).

Item 32 further specifies the mammalian cell of item 29 as being a viral packaging cell.

Item 33 provides a use of the cell of item 31 or 32 for virus production, preferably AAV production.

Item 34 provides a non-human transgenic animal comprising the DNA transposon of any one of items 1 to 13.

Item 35 provides a pharmaceutical composition comprising (a) the DNA transposon according to any one of items 1 to 13; and the recombinant transposase according to any one of items 15 to 21 or the isolated mRNA of item 27; or (b) a human cell comprising the DNA transposon of any one of items 1 to 13, wherein the cell is preferably a patient autologous cell.

Item 36 further specifies the pharmaceutical composition of item 30 for use in gene therapy or treating cancer.

Item 37 provides a method for preparing a cell comprising a stably integrated heterologous polynucleotide, comprising (a) introducing a DNA molecule comprising the DNA transposon according to any one of items 1 to 13 or the expression vector according to item 14 into a eukaryotic cell, wherein the heterologous polynucleotide comprises a sequence encoding a gene of interest, a complementary DNA (cDNA), a genome of interest or another genetic element, and wherein the DNA transposon further comprises a sequence encoding a selectable marker; (b) introducing a recombinant transposase source into said eukaryotic cell, wherein the recombinant transposase source is selected from the group consisting of: (i) the expression vector of item 26; (ii) the isolated mRNA of item 27; and (iii) the recombinant transposase of any one of items 15 to 21; and (c) culturing the eukaryotic cell in a medium under conditions to select for the selectable marker, wherein the DNA transposon is stably integrated into the genome of the eukaryotic cell.

Item 38 provides a method for preparing a protein of interest, comprising (a) introducing a DNA molecule comprising the DNA transposon according to any one of items 1 to 13 or the expression vector according to item 14 into a eukaryotic cell, wherein the heterologous polynucleotide comprises a sequence encoding at least one protein of interest and further a sequence encoding a selectable marker; (b) introducing a recombinant transposase source into said eukaryotic cell, wherein the recombinant transposase source is selected from the group consisting of: (i) an expression vector of item 26; (ii) the isolated mRNA of item 27; and (iii) the recombinant transposase of any one of items 15 to 21; (c) culturing the eukaryotic cell in a medium under conditions to select for the selectable marker, wherein the heterologous polynucleotide comprising a sequence encoding the at least one protein of interest and a sequence encoding a selectable marker is integrated into the genome of the eukaryotic cell; (d) optionally isolating a single clone for clonal expansion to prepare a monoclonal cell line; (e) culturing the eukaryotic cell under conditions to produce the protein of interest; and (f) harvesting and optionally purifying the protein of interest.

Item 39 provides a method for preparing a virus or virus like particle of interest, comprising (a) introducing a DNA molecule comprising the DNA transposon according to any one of items 1 to 13 or the expression vector according to item 14 into a eukaryotic cell, wherein the heterologous polynucleotide comprises a sequence encoding at least one virus or virus like particle of interest and further a sequence encoding a selectable marker; (b) introducing a recombinant transposase source into said eukaryotic cell, wherein the recombinant transposase source is selected from the group consisting of: (i) an expression vector of item 26; (ii) the isolated mRNA of item 27; and (iii) the recombinant transposase of items 15 to 21; (c) culturing the eukaryotic cell in a medium under conditions to select for the selectable marker, wherein the heterologous polynucleotide comprising a sequence encoding the at least one virus or virus like particle of interest and a sequence encoding a selectable marker is integrated into the genome of the eukaryotic cell; (d) optionally isolating a single clone for clonal expansion to prepare a monoclonal cell line; (e) culturing the eukaryotic cell under conditions to produce the virus or virus like particle; and (f) harvesting and optionally purifying the virus or virus like particle.

Item 40 provides a method for preparing a virus packaging cell line, comprising (a) introducing a DNA molecule comprising the DNA transposon according to any one of items 1 to 13 or the expression vector according to item 14 into a eukaryotic cell, wherein the heterologous polynucleotide comprises a sequence encoding at least one viral gene and further a sequence encoding a selectable marker; (b) introducing a recombinant transposase source into said eukaryotic cell, wherein the recombinant transposase source is selected from the group consisting of: (i) an expression vector of item 26; (ii) the isolated mRNA of item 27; and (iii) the recombinant transposase of items 15 to 21; (c) culturing the eukaryotic cell in a medium under conditions to select for the selectable marker, wherein the heterologous polynucleotide comprising a sequence encoding the at least one viral gene and a sequence encoding a selectable marker is integrated into the genome of the eukaryotic cell; (d) optionally isolating a single clone for clonal expansion to prepare a monoclonal cell line; and (e) culturing the eukaryotic cell under conditions to produce the at least one viral gene.

Item 41 further specifies the method of item 40 to comprise a further step (c1) or (d1) following step (c) or (d), respectively, of transiently transfecting the eukaryotic cell with at least one further viral gene and/or transducing the eukaryotic cell with a helper virus; and optionally (f) harvesting and optionally purifying the virus or virus like particle.

Item 42 further specifies the method of item 40 or 41, wherein the at least one viral gene is from AAV, preferably wherein the at least one viral gene is an AAV rep and/or cap gene.

Item 43 further specifies the method of item 39 wherein the at least one virus or virus like particle of interest is AAV or VSV, wherein for AAV the method may further comprising (a) transducing the eukaryotic cell with a helper virus (e.g., from HSV or AdV) or (b) transfecting the eukaryotic cell with at least one helper virus gene (e.g., from HSV or AdV).

Item 44 further specifies the method of any one of items 39 to 43, wherein the eukaryotic cell is a mammian cell.

Item 45 further specifies the method of item 37 or 44, wherein the method is an in vitro method.

### EXAMPLES

### Methods

### Plasmid Generation

The transposon plasmids for stable transfections contain a promoter-driven antibody expression cassette, the ampicillin resistance and a metabolic glutamine synthetase selection marker. Plasmids encoding the transposase were cloned and transfected together with the respective transposon. Circular plasmids are used for transfection.

### Host Cell Cultivation

The CHO-K1 host cell (harboring a genomic knockout of the endogenous glutamine synthetase gene) was cultivated in host cell medium with added L-glutamine. The cultivation of the host cell was started at a seeding density of 3×10E05 cells per mL. The growth conditions were set to 36.5° C. with and 5% CO2 in a shaking incubator with 120 rpm in shake flasks. Determination of cell density and viability took place in the Cedex HiRes© cell count analyzer.

### Transfection of Transposase and Transposon in CHO

One day before transfection, the host cells were seeded with a cell density of 0.8×10E06 cells/mL in shake Flask. On the day of transfection cell density and viability were determined and the required cell amount for transfection calculated. Cells were centrifuged for 7 minutes with 750×G, the supernatant discarded and 1-3 μg of transposon and 1-2 μg transposase plasmid DNA added per shot with the Neon©transfection system. The electroporation cuvette was filled with 3 mL of electroporation buffer E2, resuspended cells (5×10E6) with approximately 90 μl of buffer R (total volume of 100 μl) and mixed with the 4 μg of transposase and transposon plasmids. The transfection was performed using 1500 Volt, 10 mS with and a pulse of 2. Transfected cells were transferred in 5 ml prewarmed host medium in T25 ml flasks and incubated with 8% CO2 and 37° C. for at least 24 h.

Alternatively, the transposase was transfected as mRNA (1-2 μg per transfection), using the same procedure.

### Selection of Stable CHO Pools

24 h after transfection, cells were transferred into selection conditions (medium does not contain L-glutamine). 10 mL of selection medium was prewarmed for every pool in T75 Flasks. Two stable pools were cultivated for each transfection.

Cells were centrifuged (for 7 minutes with 750×G) and the supernatant was discarded. Cells were resuspended with 20 mL of selection medium and incubated with 8% CO2 at 37° C. During selection cells were monitored by microscopy. Additional medium (5 mL) was added after 3-5 days.

### Passaging of Stable Pools

Once cells reached a viability of at least 70% and a doubling time of at least 48 hours, the selection phase was considered successful. After selection, the cells are passaged starting at 3×10E05 cells per mL in 30 mL total volume in 125 mL shake flask at 36.5° C. and 5% CO2 at 120 rpm shaking. Samples for titer measurements are taken regularly. Antibody titer was measured using a ForteBio Octet device with protein A Biosensors.

Copy Number Determination Via ddPCR

For determination of transgene copy number in the stable pools the ddPCR (digital droplet PCR) method was applied. Genomic DNA was isolated via QiaSymphony from washed and centrifugated cells (0.5×106 cells per sample) of the stable pools.

300 ng of sample genomic DNA was digested with one or two restriction enzymes (according to manufacturer's protocol), which cuts the antibody cassettes at both ends. Restriction enzyme digestion was conducted for one hour at 37° C. Subsequently samples were diluted 1:10 in nuclease free water. For the copy number determination two reactions per sample were prepared. One with a primer probe set for the heavy chain and one with a primer probe set for the light chain. The house keeping gene Eif3i was used for normalization of DNA amounts. Droplets were generated on the automated Droplet Generator (Biorad) followed by PCR. The analysis and evaluation were performed with the Droplet reader (Biorad) and the QuantaSoft software (Biorad).

Relative Gene Expression Via ddPCR

For determination of relative transgene expression in the stable pools the ddPCR (digital droplet PCR) method was applied. Total RNA was isolated via QiaSymphony from washed and centrifugated cells (0.5×106 cells per sample) of the stable pools. Isolated RNA was diluted and 0.5 ng were used to set up the reaction mix which combines components for both the reverse transcription of the RNA and the subsequent amplification of cDNA. Both reactions were executed subsequently in the same tube with an optimized PCR protocol. The house keeping gene Eif3i was used for normalization of DNA amounts. Droplets were generated on the automated Droplet Generator (Biorad) and subsequently the RT-PCR followed by cDNA PCR reaction in one run. The analysis and evaluation was performed with the Droplet reader (Biorad) and the QuantaSoft software (Biorad).

### Fed-Batch Bioprocess Assessment

Bioprocess performance of monoclonal antibody (IgG1) producing stable pools was assessed under regular fed-batch cultivation conditions in an advanced micro bioreactor system (ambrTM15, Sartorius). The process duration was 14 days and feeding was initiated on day 2 post inoculation. Cell were seeded at day 0 at a density of 0.7×106 viable cells per mL and cell culture temperature was set to 34.5° C. Cell culture parameter were monitored daily (product titer determination was started on day 9). Culture pH was set to 6.95±0.25 and glucose concentration was increased to 6 g/L if concentrations dropped below 4 g/L.

### Cell Surface Staining and Flow Cytometry Analysis

NRP1 expression and display on the cell surface was assessed by cell surface staining with a fluorochrome-labelled anti-NRP1 antibody and subsequent analysis via flow cytometry. For the cell staining 0.5×105 cells per sample were washed, resuspended in a PBS-based buffer containing BSA, EDTA and sodium azide and subsequently transferred to a 96-well V-bottom plate. The plate containing the cells was centrifuged (5 min, 300×g) and PBS-buffer was replaced with staining solution containing the anti-NRP1 antibody or an isotypic control. The host cell negative control was incubated with PBS-buffer. Samples were incubated at 4° C. for 45 min protected from light. Stained cells were again washed with PBS-buffer and subsequently analysed with a NovoCyte flow cytometer (Agilent). For the analysis the cell population of interest was evaluated with a scatter plot of FSC-H vs. SSC-H. Signal intensity of bound antibody was determined through the coupled APC fluorochrome in a APC-H histogram. The threshold for APC-positive cell discrimination was set with the stained untransfected host cell sample and was kept constant for all subsequent measurements.

### Example 1: Identification of a Novel Transposase/Transposon Pair from Acyrthosiphon pisum

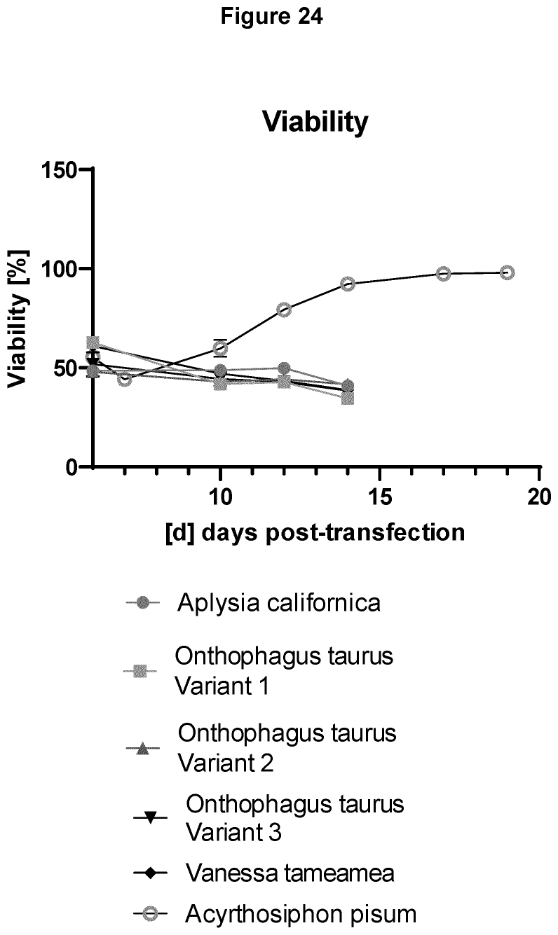

We describe the identification of a novel transposase/transposon pair from Acyrthosiphon pisum (AP). Based on published genomic sequences, 384 potential different transposase/transposon-like sequences were identified. Sequences were screened for repetitive, ITR-like elements 3′ and 5′ of a potential transposase sequence in the genomic proximity. Moreover, the potential transposase sequences were assessed for catalytic amino acid residues (DDE-motif). 14 transposase/transposon-like sequences were experimentally tested and only one sequence pair (identified from genomic sequences from Acyrthosiphon pisum) surprisingly demonstrated active transposition in eukaryotic (Chinese hamster ovary, CHO) cells (for selected results see FIG. 24 and Example 17). Activity of the transposase was demonstrated in transfection experiments using circular plasmid DNA vectors as well as mRNA coding for the AP transposase.

The novel transposase/transposon pair from Acyrthosiphon pisum has to the best of our knowledge not been described before. The novel transposase/transposon pair from Acyrthosiphon pisum presents itself as an additional, orthogonal transposase/transposon pair, that is functionally active in eukaryotic cells. The transposase is specifically recognizing the discovered transposon ends, which are substantially different from any published transposase/transposon system, and transposition leads to the stable integration of any cargo gene into the host cell genome of interest.

### Example 2: Identification and Activity Test in CHO Cells

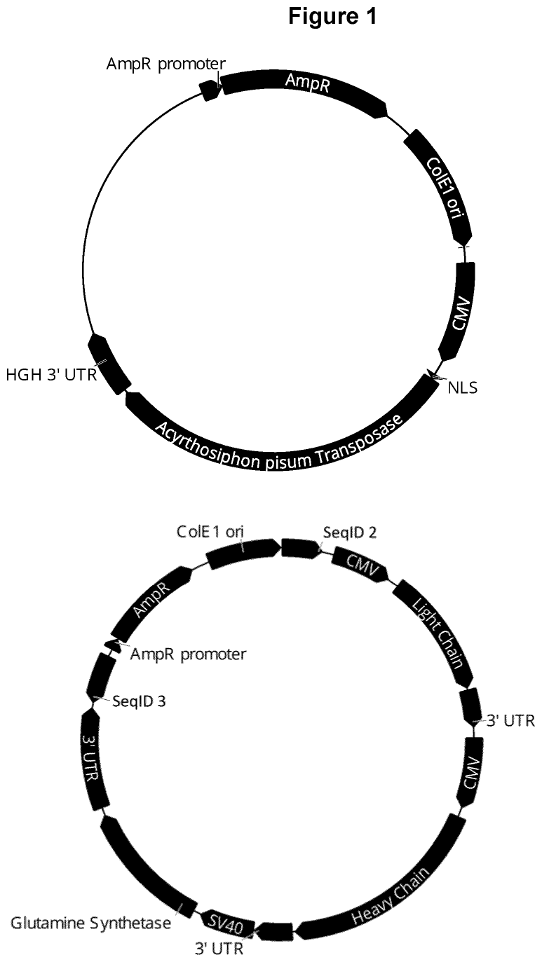

It has been experimentally demonstrated that the novel transposase/transposon pair from Acyrthosiphon pisum (NCBI Reference Sequence: XP_029340918.1) is active in CHO cells, resulting in transposition of a transposon coding for flanking sequences and ITRs as well as a monoclonal antibody expression cassette into the genome of the host cell. CHO-K1 GS−/− cells (harbouring a biallelic knockout of the endogenous glutamine synthetase gene) were transfected with a transposon vector encoding for a metabolic selection marker (glutamine synthetase) and an expression cassette of a therapeutic monoclonal antibody under the control of a CMV promoter. The transposon vector had ITR core sequences (Seq ID NO: 1) within both flanking sequences, left flanking sequences of Seq ID NO: 2 and right flanking sequences of Seq ID NO: 3. Additionally, a second plasmid coding for the Acyrthosiphon pisum transposase (Seq ID NO: 4) with a heterologous N-terminal nuclear localization signal (Seq ID NO: 5, full sequence SEQ ID NO: 6) under the control of a CMV promoter was transfected and stable cells were selected in cell culture medium lacking L-glutamine (FIG. 1).

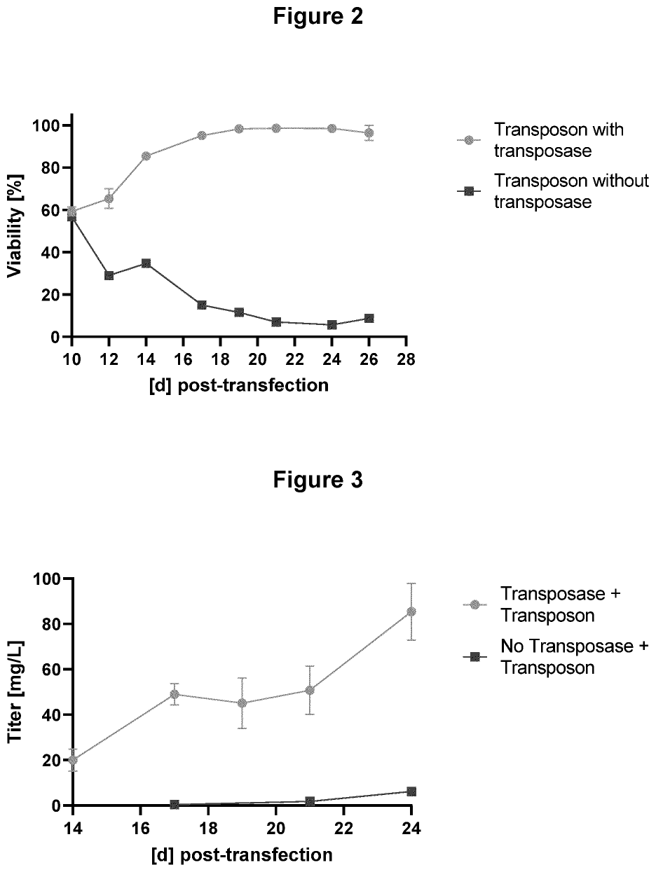

As a control, CHO K1 GS−/− cells were transfected with the same transposon vector but without the transposase plasmid. The viability of the transfected cells was measured for 18 days post transfection (FIG. 2). CHO cells transfected with transposon and transposase showed successful transposition and thus stable integration of the transposon into the genome of the CHO cells, by recovering from the selection process after approximately 12 days to viabilities >70%. The viability further increased to stable levels >90%, while the negative control (transfected with transposon only) cells did not recover from the selection experiment.

The stable transfection of an antibody expression cassette as the transgene transposon cargo resulted in highly productive and stable recombinant CHO cell lines (FIG. 3), which would be highly suitable for the manufacturing of therapeutic proteins. Established stable cell lines showed higher antibody titers and an increased antibody expression stability compared to pools established by random transgene integration. Negative control cell samples lacking the transposase plasmid, did not result in productive stable cell pools.

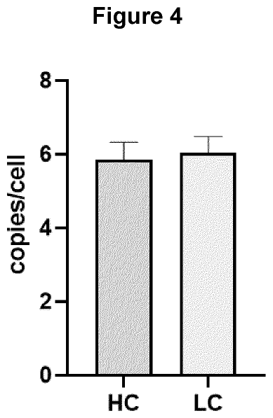

Random integration of transgenes often results in concatemerisation, fragmentation, rearrangements and recombination of transgenes leading to a disbalance of transgene copies and partial loss of transgene or selection marker copies in the genome of recombinant stable cell lines. On the other hand, transposase-mediated stable integration of transgenes follows a cut and paste mechanism, leaving single intact copies of the transposon cargo at distinct genomic loci in the resulting recombinant stable cell lines. Genetic analysis of the stable CHO pools co-transfected with AP transposase and transposon showed a highly balanced copy number for the heavy and light chain gene of the therapeutic monoclonal antibody (FIG. 4).

### Example 3: Influence of Heterologous Nuclear Localization Signals on Transposition Efficiency

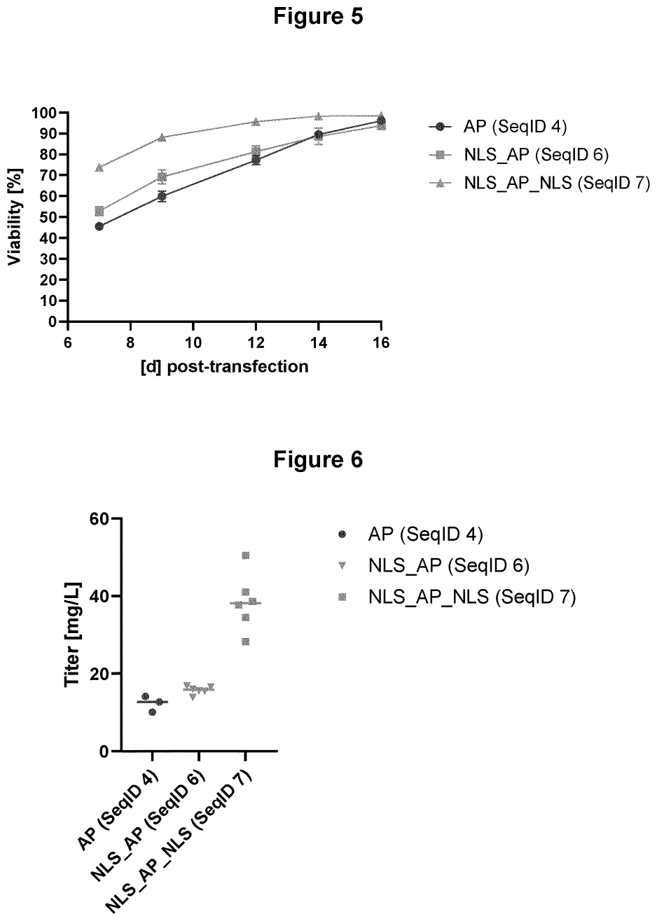

We investigated the influence of adding additional heterologous nuclear localization signals to the AP transposase gene at the C- and N-terminus and studied the efficiency of transposition and generation of stable recombinant CHO cell pools expressing therapeutic antibodies. A transposon plasmid encoding for a therapeutic antibody under the control of a CMV promoter, a SV40 controlled expression of a glutamine synthetase gene as a metabolic selection marker and flanking sequences of Seq ID NO: 2 and Seq ID NO: 3 (including ITR sequences of Seq ID NO: 1), as well as an AP transposase plasmid were co-transfected. Initial sequence analysis indicated that the naturally occurring transposase gene already exhibits an NLS-like sequence at the N-terminus (Seq ID NO: 25). We investigated AP transposase genes without an additional N-terminal NLS (Seq ID NO: 4), with one additional N-terminal NLS (Seq ID NO: 6) or two NLS motifs at both the N- and C-terminus (Transposase: Seq ID NO: 7; NLS Seq ID NO: 22 (N-terminal) and Seq ID NO: 14 (C-terminal)). All constructs successfully survived the selection phase, indicating active transposition of the transposase/transposon combination resulting in a stable integration of the antibody and selection marker expression cassettes into the genome of the CHO host cell. The construct harboring two NLS motifs (Seq ID NO: 7) reached viability levels >70% already after 7 days of selection, being significantly faster than the other constructs with Seq ID NO: 4 and Seq ID NO: 6, which reached viability levels >70% only after 12 and 9 days respectively (FIG. 5).

These experiments showed that by increasing the number of NLS motifs and attaching these sequences N- and C-terminally, an increase in transposition efficiency of the transposase/transposon system was observed, as indicated by faster outgrowth of stably transfected cells from the metabolic selection. This could be driven by increased nuclear import of the transposase enzyme and/or higher expression rates. Furthermore, titer measurements confirmed that an AP transposase construct harboring two NLS motifs resulted in pools with higher productivity and an approximately 2-fold improvement compared to transposase constructs lacking additional nuclear localization signal (FIG. 6).

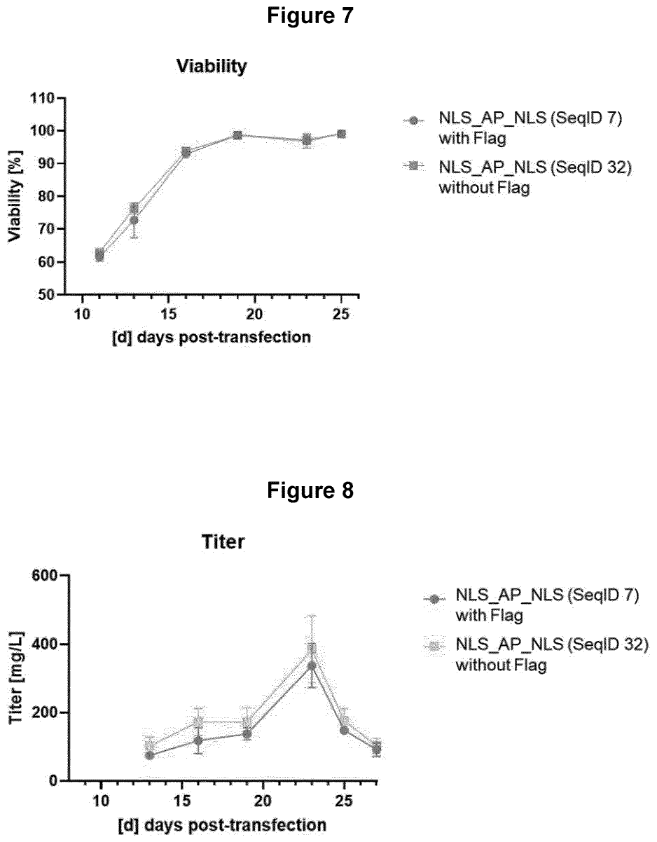

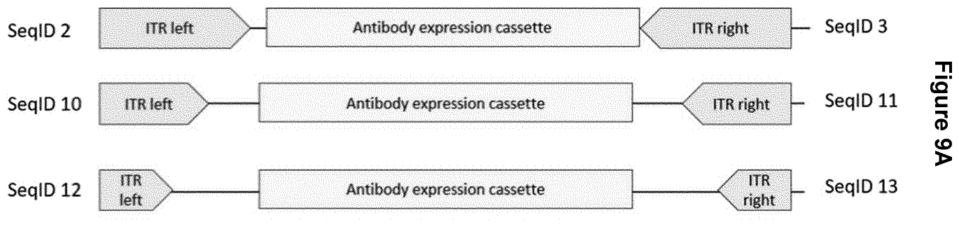

Furthermore, we investigated whether the presence of the Flag-tag itself as used in the construct having the sequence of SEQ ID NO: 7 had an impact on the transposition activity of the AP transposase. In a head-to-head comparison, stable pools transfected with SEQ ID NO: 7 and SEQ ID NO: 32 (lacking the Flag-tag) showed comparable performance, indicating that the Flag-tag did not influence the performance and transposition activity of the AP Transposase protein (FIG. 7 and FIG. 8).

### Example 4: Investigation of Flanking Sequences

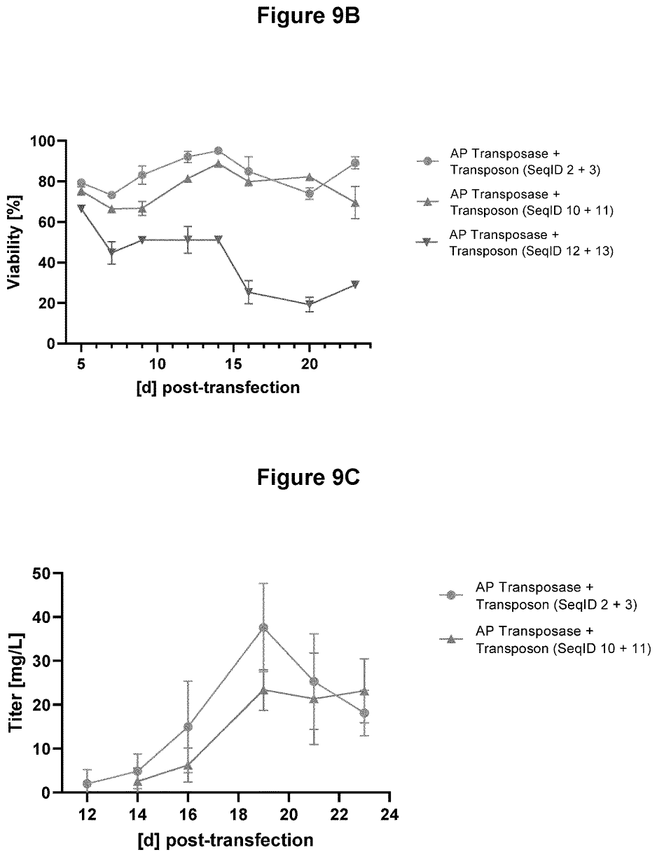

The flanking sequences (Seq ID NO: 2 and Seq ID NO: 3) are essential for the AP transposase to recognize the transposon and induce stable genomic integration via transposition. Flanking sequences were extracted from proximal genomic sequences 3′ and 5′ of a transposase gene. To identify the minimal, functional flanking sequence motif, a set of truncated flanking sequences were designed (SeqID 2, 3, 10, 11, 12, 13) and tested for transposition in CHO cells. All truncated flanking sequences harboured ITR sequences of Seq ID NO: 1 in the left (5′) flanking region or the reverse complementary ITR of SEQ ID NO: 15 in the right (3′) flanking region (FIG. 9A).

CHO cells were transfected with a plasmid encoding AP transposase and a transposon encoding an expression cassette of a therapeutic antibody with varying flanking sequence combinations (see FIG. 9A). Flanking sequence combinations SeqID2+3 (SEQ ID NO: 2 and SEQ ID NO: 3) and SeqID10+11 (SEQ ID NO: 10 and SEQ ID NO: 11) survived the selection process, indicating active transposition events and a functional transposase/transposon combination (FIGS. 9B and C). Flanking sequence combination SeqID12+13 (SEQ ID NO: 12 and SEQ ID NO: 13) remained unfunctional (FIG. 9B).

Stable and productive CHO pools could only be generated for functional transposons SeqID2+3 and SeqID10+11, where both transposon set-ups reached comparable titers (FIG. 9C).

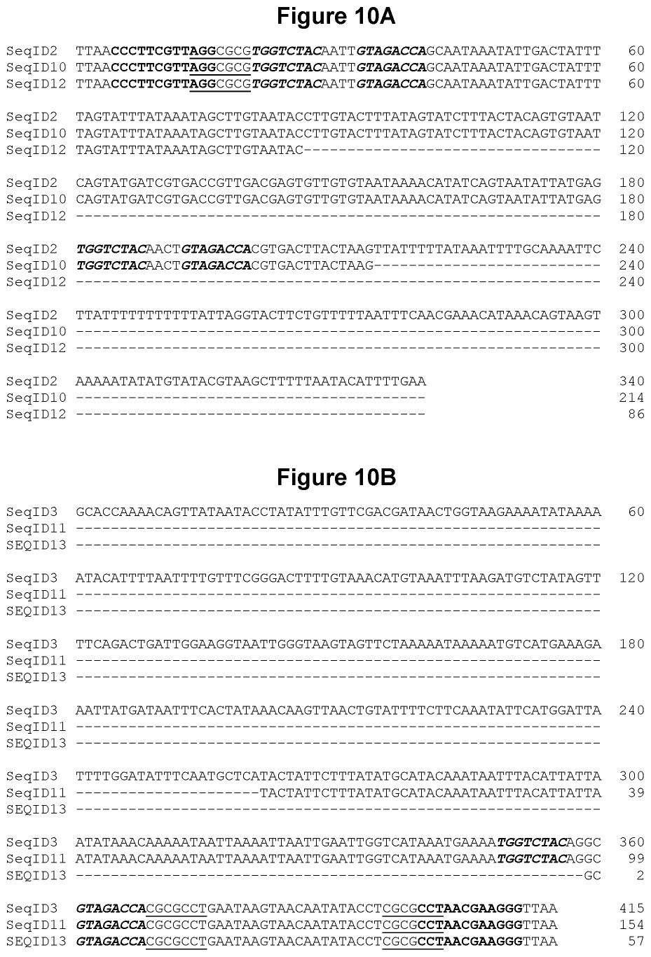

Sequence alignment of left and right flanking sequences is shown in FIG. 10A and B. The flanking sequences were further analyzed for internal repetitive motifs. Functional flanking sequences exhibit further at least one internal repeat motif (IR) (tggtctac) and its inverted complementary sequence separated by 4 nucleotides (together referred to as inverted internal repeat (IIR)), wherein at least the first of these nucleotides is an adenosine (SEQ ID NO: 43). Conserved sequences of SEQ ID NO: 43 (or more specifically SEQ ID NO: 44) were identified in both flanking arms, two in the left flanking arm and one in the right flanking arm.

It is therefore likely that the right flanking region needs to comprise at least one IR (tggtctac) and its inverted complementary sequence separated by 4 nucleotides, i.e., comprising at least from nt 349 to nt 415 of SEQ ID NO: 3 or from nt 88 to nt 154 of SEQ ID NO: 11, or SEQ ID NO: 47, 48 or 49. In the left transposon flanking arm, both IIRs seem to be important. It is therefore postulated that the left flanking region needs at least the sequence of SEQ ID NO: 40, 45 or 46 and further an inverted internal repeat motif having the sequence of SEQ ID NO: 43 separated by a flexible stretch of nucleic acids, such as about 50 to 200 nucleotides.

### Example 5: Further Investigation of Flanking Sequences

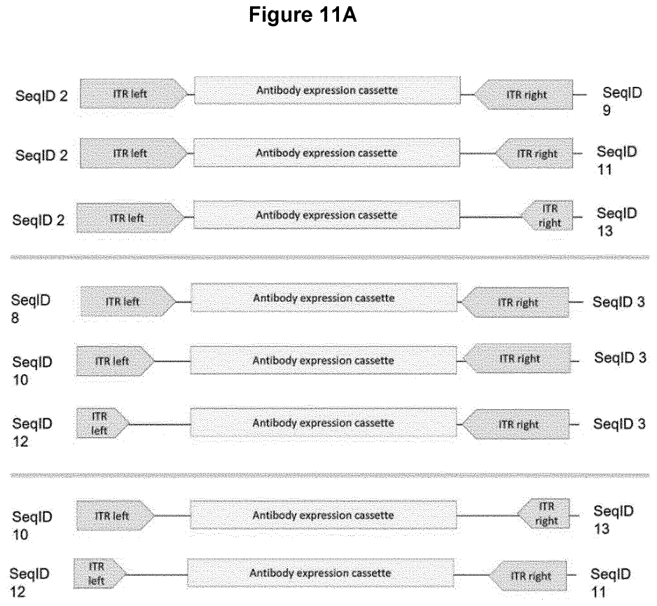

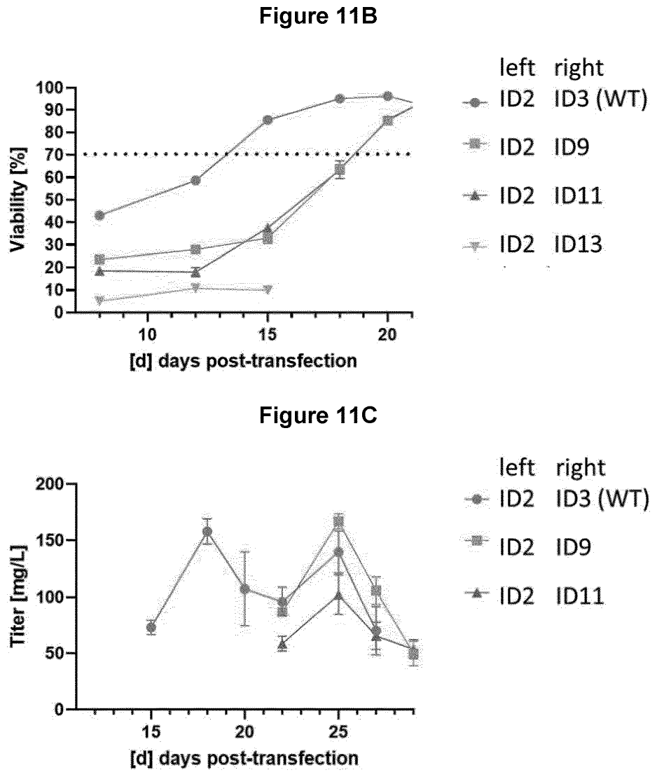

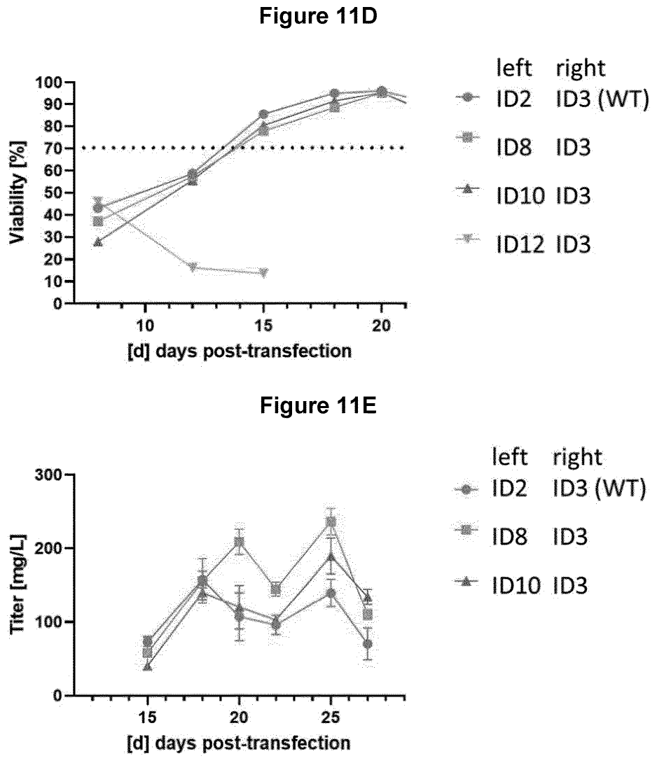

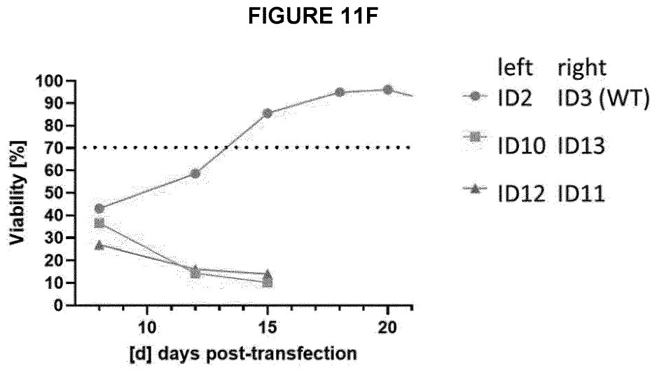

In a further experiment either the right or left flanking region was truncated to better understand the individual influence and minimal motif (FIG. 11A).

CHO cells were transfected as described in Example 4 using a transposon encoding an expression cassette of a therapeutic antibody with constant full length left flanking AP sequences (SEQ ID NO: 2) and truncated right flanking AP sequences (SEQ ID NO: 3 (WT), SEQ ID NO: 9, SEQ ID NO: 11 and SEQ ID NO: 13, respectively) (FIG. 11A, top and FIG. 11B and C) orwith truncated left flanking AP sequences (SEQ ID NO: 2 (WT), SEQ ID NO: 8, SEQ ID NO: 10 and SEQ ID NO: 12, respectively) and constant full length right flanking AP sequences (SEQ ID NO: 3) (FIG. 11D and E. SEQ ID NO: 8 is an intermediate truncated left transposon flanking sequence of 297 nucleotides, i.e., between SEQ ID NO: 2 (340 nt) and SEQ ID NO: 10 (214 nt) and SEQ ID NO: 9 is an intermediate truncated right transposon flanking sequence of 260 nucleotides, i.e., between SEQ ID NO: 3 (415 nt) and SEQ ID NO: 11 (145 nt).

In a first experiment the left flanking sequence (SEQ ID NO: 2) was held constant, while the right flanking sequences were systematically truncated (SEQ ID NOs: 9, 11 and 13). Selection behavior and productivity of the stable pools were compared to the AP wildtype control with full length flanking sequences (SeqID2 and SeqID3). When systematically truncating the right flanking sequence to SeqID9 or SeqID11, the selection duration is prolonged significantly compared to wildtype control, while overall productivity remains unchanged (FIG. 11B and C). Further reducing the right flanking sequence to SeqID13 leaves the transposon dysfunctional with cells not recovering from the metabolic selection (FIG. 11B). Alignment of right flanking sequences (SEQ ID NOs: 3, 11 and 13 in FIG. 10B) with annotated repeat and binding motifs, suggests that the depletion of one of the internal repeats in the IIR motif in SEQ ID NO: 13 is detrimental to the transposon function. Furthermore, the full length right transposon flanking region (SEQ ID NO: 3) seems to be superior to all truncated right flanking sequences.

In a second experiment the right flanking sequence (SEQ ID NO: 3) was held constant, while the left flanking sequences were systematically truncated (SEQ ID NOs: 8, 10 and 12). Selection behavior and productivity of the stable pools were compared to the AP wildtype control with full length flanking sequences (SEQ ID NOs: 2 and 3). When systematically truncating the left transposon flanking sequence to SeqID8 or SeqID10, the selection duration and overall productivity remains unchanged, suggesting that as long as both IIR motifs remain intact, no impairment of the transposon function is expected (FIG. 11D and E). Further reducing the left flanking region to SEQ ID NO: 12 leaves the transposon dysfunctional with cells not recovering from the metabolic selection (FIG. 11D). FIG. 10A shows the alignment of left transposon flanking sequences with annotated repeat and binding motifs, suggesting that the depletion of one of the IIR motifs in SEQ ID NO: 12 abandons the function of the transposon flanking region.

As expected, when combining truncated forms of the left and right flanking sequence (left: SeqID: 10, 12; right: SEQ ID NO: 13, 11) it leaves the transposon dysfunctional with cells not recovering from the metabolic selection (FIG. 11F).

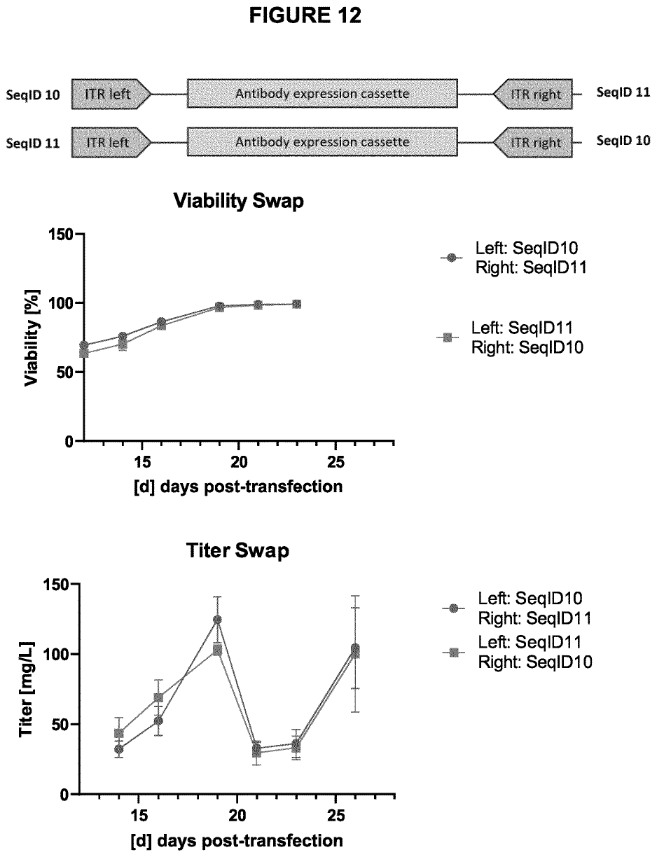

Further it was analysed whether swapping left and right transposon sequences would have an effect on selection and productivity. For this experiment, a transposon encoding for an antibody expression cassette was transfected using SEQ ID NO: 10 and SEQ ID NO: 11 as left and right transposon sequences, respectively and a second transposon comprising the inverse order were tested (FIG. 12, top). Cell culture performance was assessed by monitoring the selection phase as well as determining productivity. As may be taken from FIG. 12 (middle and bottom) the orientation of the flanking sequences can be swapped resulting in similar cell culture performance.

### Example 6: Investigation of Motif Alterations in Flanking Regions

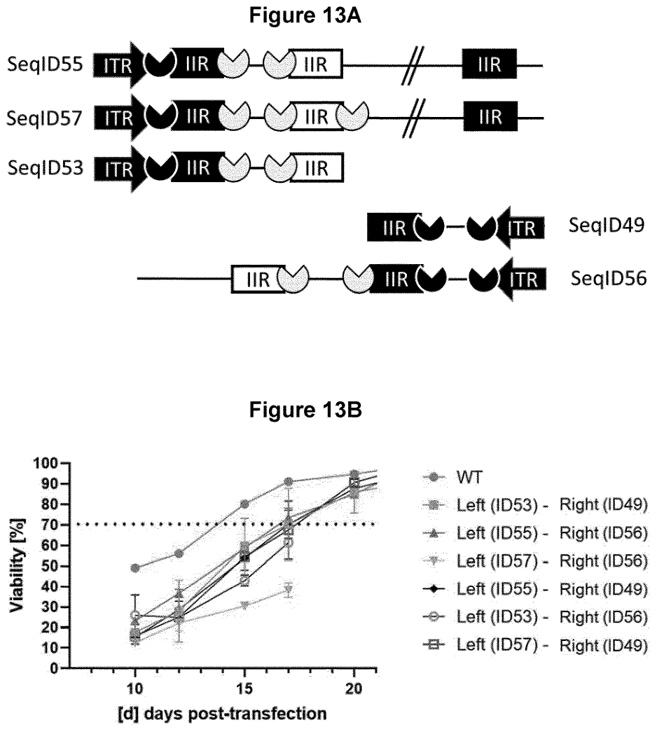

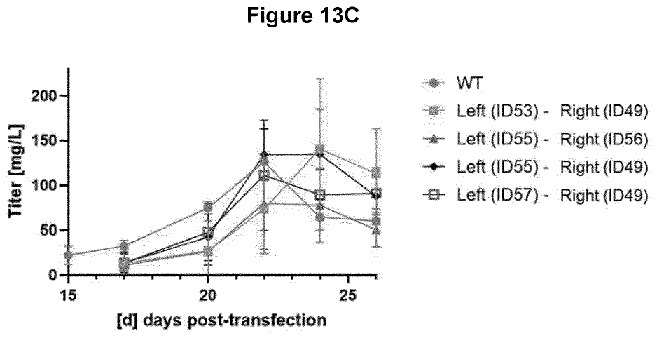

The previous experiments have shown that binding and repeat motifs found in the AP transposon flanking regions play a crucial role in mediating successful und efficient transposition. Further, the flanking regions were modulated by artificially introducing additional motifs as illustrated in FIG. 13A to investigate their impact on selection behavior and productivity.

Based on the finding that removal of the IIR motif from the left flanking region (see FIG. 10A, SEQ ID NO: 12) results in impaired selection behavior (see FIG. 11D) different modified left flanking regions were designed (FIG. 13; SEQ ID NO: 53 (SeqID53), SEQ ID NO: 55 (SeqID55) and SEQ ID NO: 57 (SeqID57)). The first design (SeqID55) is a modified construct that contains an additional binding motif and binding-IIR motif setup taken from the right AP transposon flanking region (SEQ ID NO: 54, corresponding to nt 1-54 of SEQ ID NO: 49 and nt 349-402 of SEQ ID NO: 3), as it contains two additional binding motifs and provides a certain distance to the native motifs to avoid potential sterical effects. In the second design (SeqID57) the construct was further complemented with an additional binding motif following the newly introduced IIR in an attempt to introduce additional changes in a modular concept. The third design (SeqID53) picks up the concept of a minimal required length for the flanking region from previous experiments and the knowledge thereof about the significance of the native second IIR motif. This design (SeqID53) is a truncated variant of SeqID55 containing only the ITR, first native binding and IIR motif as well as the artificially added binding motif and binding-IIR motif (SEQ ID NO: 54) originating from the right flanking region as in SeqID55, but terminating right after this artificially introduced IIR. This setup will show whether the artificially added motifs can rescue impairment of selection behavior and productivity caused by removal of the second native IIR (SeqID12) as shown in FIG. 11D.

All left flanking region designs were combined with a minimal right flanking region variant (SEQ ID NO: 49 or SeqID49) with the intention to firstly confirm that this minimal sequence is functional and secondly to generate a baseline for subsequent changes in the right flanking region.

The results show that all introduced modifications led to a significantly impaired metabolic selection behavior (FIG. 13B) compared to the wildtype flanking region setup (SeqID2 and SeqID3). In contrast, the overall productivity remained mostly unchanged (FIG. 13C). The effects of SeqID55 and SeqID57 were comparable with a slight downswing on the SeqID57 side using the modular concept approach. Interestingly, the minimal right flanking region setup proved to be functional in all tested combinations. Especially in combination with the minimal setup of SeqID53 the productivity was highly comparable to the wildtype. This also demonstrates that the artificially introduced motifs of SeqID53 had a partial rescue effect on the removal of the second native repeat motif.

In a following step the above tested left flanking region modifications (SeqID53, SeqID55 and SeqID57) were combined with a modified right flanking region (SeqID56) which contained a further copy of the native binding and binding-IIR motif unit (SEQ ID NO: 54) to test the influence of a second IIR motif being present in the right flanking sequence. Only the combination with SeqID55 resulted in similar metabolic selection behavior compared to the minimal sequence of SEQ ID NO: 49 whereas the other combinations with SEQ ID NO: 53 and SEQ ID NO: 57 displayed drastically impaired recovery from selection (FIG. 13B). With regards to productivity the modified right flanking region SEQ ID NO: 56 stayed below wildtype and SEQ ID NO: 49 combinations (FIG. 13C). In conclusion, the right flanking regions exhibit high sensitivity to alterations in the binding and repeat motif setup reflected in detrimental effects on selection behavior and productivity. However, it seems that the minimal right flanking region is effective and sufficient for transposition.

### Example 7: Combination with Glutamate Synthetase from Providencia Vermicola

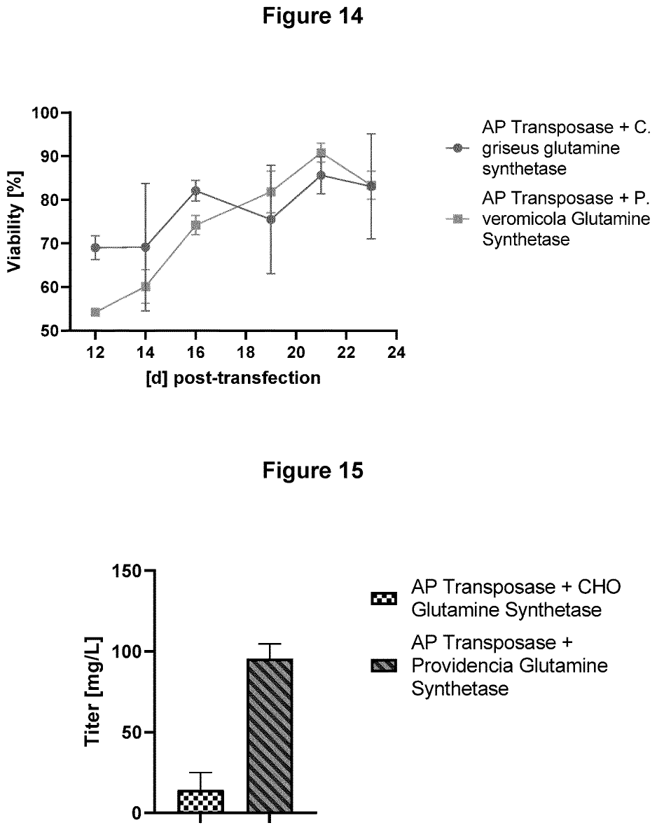

The AP transposase/transposon was tested with an improved transposon set-up, harboring an attenuated bacterial glutamine synthetase selection marker from Providencia vermicola as described previously in EP 22163849.7. Selection behavior was comparable between both tested metabolic selection markers (FIG. 14).

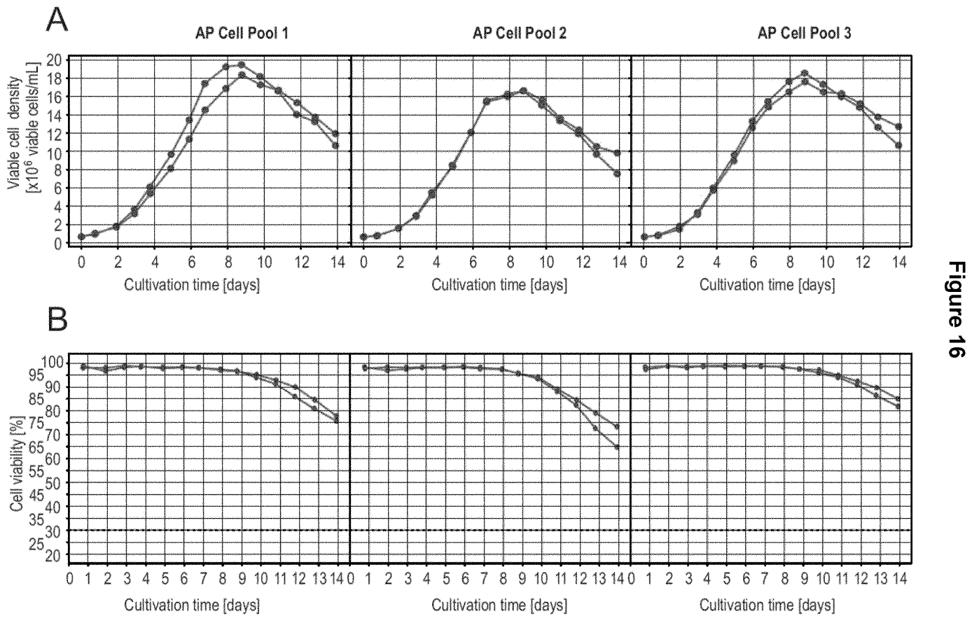

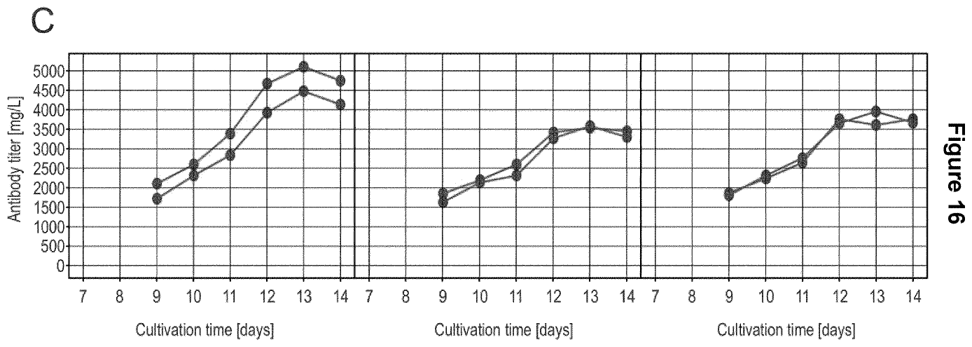

Surprisingly, stable CHO pools transfect with AP transposase and a transposon harboring a bacterial glutamine synthetase selection marker showed an approximately 7-fold increase in recombinant antibody titer (FIG. 15). This demonstrates that the AP transposase and the respective transposons can also be used with a different more stringent metabolic selection marker.

### Example 8: Bioprocess Performance of Stable Pools Generated Via AP Transposase

To investigate the cell culture performance of stable CHO cells expressing a recombinant protein, stably selected pools were cultivated in a controlled bioreactor system under fed-batch process conditions.

Towards this end, CHO-K1 cells were co-transfected with the AP transposase and a transposon encoding the heavy and light chain genes of a monoclonal antibody in combination with an attenuated bacterial glutamine synthetase selection marker from Providencia vermicola. For production bioreactor assessment, three different stable recombinant monoclonal antibody expressing CHO cell pools were inoculated to the bioreactor (N=2 per pool) and cultivated for 14 days. From day two onwards, a nutrient feed solution was added daily to the cultures and cell culture parameters including cell growth, viability and monoclonal antibody titer were monitored until day 14.

Stable monoclonal antibody expressing CHO cell pools generated using the AP transposase showed very good and robust bioprocess performance reaching final product titers of up to 5 g/L (FIG. 16). In addition, all three stable pools displayed a similar culture behavior with regards to cell growth, viability and product titer formation indicating a high level of robustness and reproducibility.

### Example 9: Bioprocess Performance of Stable Pools Generated Via AP Transposase Variants

To further improve the AP transposase protein, novel protein variants were engineered and tested for enhanced activity based on the wildtype AP transposase with N- and C-terminal NLS (nucleic acid sequence: SEQ ID NO: 31; amino acid sequence: SEQ ID NO: 32). CHO-K1 cells were transfected as described and the selection phase was monitored. Table 1 summarizes the tested protein variants, which were mainly designed based on a classic family shuffling approach.

TABLE 1
Tested AP transposase variants.

| AP transposase mutation | Selection behavior | Productivity |
|---|---|---|
| E428Q | N/A | N/A |
| K87Y | ++ | + |
| H100E | N/A | N/A |
| A264S | 0 | 0 |
| F287L | N/A | N/A |
| F238K | N/A | N/A |
| Q445E | N/A | N/A |
| L25V | N/A | N/A |
| N40T | N/A | N/A |
| L123F | N/A | N/A |
| I168F | N/A | N/A |
| V258I | N/A | N/A |
| A264G | N/A | N/A |
| S270P | −− | 0 |
| Q273V | + | 0 |
| V212I + I215L | + | + |
| S277N + L284I | 0 | 0 |
| F345I + I348A | N/A | N/A |
| I363V + K365S | ++ | + |
| F76H + L78T + G79S | N/A | N/A |
| Q261M | − | −− |
| I386V | N/A | N/A |
| E391K + Y393I | N/A | N/A |
| I438A | −− | −− |
| K87Y + Q273V | + | + |
| K87Y + A264S + Q273V | ++ | 0 |
| A264S + Q273V | ++ | + |
| K87Y + A264S + S270P + Q273V | − | − |
| A264S + S270P + Q273V | 0 | 0 |
| S270P + Q273V | ++ | 0 |
| K87Y + A264S | ++ | + |
| L583F | + | + |
| K576I | + | 0 |
| Delete N584 + E585 | ++ | + |
| I578F | 0 | − |
| K206D + R209D | −− | N/A |
| N333C + A338C | −− | N/A |
| S372E | 0 | + |
| Q492K | 0 | − |
| S277N | 0 | + |
| T306K | 0 | − |
| A338F | −− | N/A |
| D577F | − | 0 |

N/A: variant dysfunctional
++: variant with strongly improved characteristics as compared to AP wild type
+: variant with improved characteristics as compared to AP wild type
0: comparable to AP transposase wild type.
−: variant with decreased characteristics compared to AP wild type
−−: variant with strongly decreased characteristics compared to AP wild type

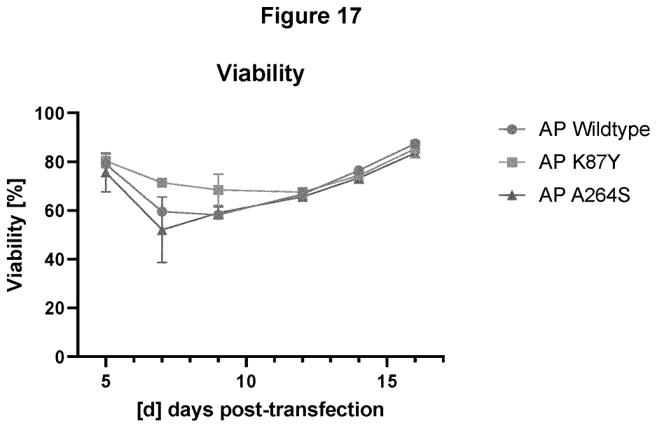

Most variants were dysfunctional and did not result in stable (--), antibody expressing CHO pools. Protein variant A264S remained functional with comparable performance as compared to AP wildtype (Table 1 and FIG. 17). Surprisingly, protein variant K87Y (nucleic acid sequence: SEQ ID NO: 33; amino acid sequence: SEQ ID NO: 34) showed improved selection and productivity behavior. Cells transfected with K87Y AP transposase grew faster from selection and reached viabilities >70% in 9 days, compared to 12 days of AP transposase wild type (FIG. 17), which was accompanied by an increased productivity (Table 1). Similar results were observed for I363V/K365S AP transposase (SEQ ID NO: 35) and K87Y/A264S AP transposase (SEQ ID NO: 37) compared to wild type (Table 1). Although not to the same extent improved performance (selection and/or productivity) was also observed for AP transposases carrying the following mutations: Q273V, V212I/I215L, L583F, K576I, S372E and S277E (Table 1). It is expected that combinations may further improve performance resulting in even more hyperactive AP transposases. For example, A264S/Q273V AP transposase (SEQ ID NO: 36) was shown to be more effective than the single mutations alone, while combinations of K87Y with Q273V seemed to rather reduce the effect of K87Y alone (K87Y/Q273V and K87Y/A264S/Q273V). Surprisingly, also deletion of the two final amino acids (N584/E585) resulted in improved selection and productivity behavior.

The mutations L582F, K576I, 1578F and D577F and deletions N584 and E585 were predicted to increase hydrophobicity of the C-terminus thereby improving dimerization. At least L582F and deletions N584 and E585 seem to have a positive effect on selection behavior as well as productivity and further K576I seems to have a positive effect on selection behavior. Further, S372E and Q492K were predicted to increase DNA binding and S277N and T306K were predicted to optimize the catalytic domain. At least S372E and S277N seems to have a positive effect on productivity without affecting the selection behavior.

We note in this regard that the wild type AP transposase shows a comparable transposition efficacy to other commercially available transposases, which are typically hyperactive variants (e.g., of piggy-BAC or sleeping beauty transposase). We therefore postulate that the hyperactive variants of AP transposase described herein and further improved variants of AP transposase, such as obtained by combining mutations, have the potential to result in transposases with even higher transposition efficacy.

### Example 10: Identifying Further AP Transposase Variants with Improved Activity Profile

Further protein engineering and mutagenesis were conducted to improve on the activity profile of AP variants using a cellular assay. Primarily, selection behavior (duration to recover from metabolic selection to reach viabilities of at least 70%) and productivity during passaging of the cells were investigated. Additional variants and particularly combination of mutations were found to increase either selection behavior or selection behavior and productivity. Both parameters are advantageous on its own and variants with both parameters improved are particularly promising.

TABLE 2
Tested AP transposase variants with
various combinations of mutations.

| | Selection | |
|---|---|---|
| | behavior | Productivity |
| K87Y + V212I + I215L | ++ | 0 |
| K87Y + Q273V + V212I + I215L | 0 | + |
| K87Y + V212I + I215L + I363V + K365S | ++ | 0 |
| K87Y + I363V + K365S | + | 0 |
| V212I + I215L + I363V + K365S + Q273V | ++ | 0 |
| I363V + K365S + Q273V | ++ | + |
| K87Y + A264S + Q273V + L583F | + | + |
| K576I + L583F | 0 | 0 |
| K576I + L583F + Del N584 + E585 | ++ | 0 |
| K87Y + A264S + Q273V + K576I + L583F | 0 | + |
| K87Y + A264S + Q273V + K576I + L583F + | ++ | + |
| Del N584/E585 | | |
| V212I + I215L + I363V + K365S | + | + |

++: variant with strongly improved characteristics as compared to AP wild type
+: variant with improved characteristics as compared to AP wild type
0: comparable to AP transposase wild type.
−: variant with decreased characteristics compared to AP wild type
−−: variant with strongly decreased characteristics compared to AP wild type

Except for one variant (K576I+L583F) the further combinations of mutations resulted in an improvement in at least one of selection behavior or productivity.

### Example 11: Generation of Bioassay Cell Line Stably Expressing Cell Surface Receptor

The novel AP transposase system was further used to generate a stable cell line for application in cell-based bioassays using expression of a transmembrane receptor. The experiment was aiming for providing more insight into the capabilities of the AP transposase system to enable expression of various types of molecules other than antibodies or secreted proteins in general. For generation of the bioassay cell line the CHO K1 host cells were co-transfected with a vector containing the gene sequence for the cell surface receptor Neuropilin-1 (NRP1) and the vector encoding the AP transposase. Subsequently, the transfected cells were subjected to the selection process to generate a stable cell pool. To evaluate the efficiency of transgene integration the average gene copy number of the cell pool was determined by isolating genomic DNA and performing droplet digital PCR (ddPCR). The same method concept was applied to isolated RNA for determination of transgene expression. In a last step the successful translation of the receptor protein and its presentation on the cell surface was evaluated via flow cytometry. Cell surface staining for flow cytometry analysis of NRP1 presentation was performed with a fluorochrome-labelled anti-NRP1 antibody. The transfected cell pools were compared to non-transfected host cell samples to identify levels of NRP1 on the cell surface.

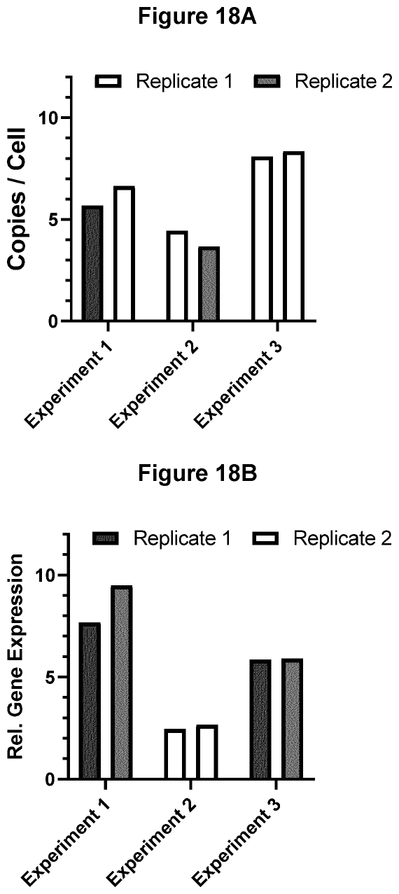

Stable cell pools were generated in three unique experiments with duplicates transfected within each experiment. Analysis of gene copy number showed a highly homogeneous pattern for both the duplicates within an experiment as well as across all experiments with final copy numbers ranging between 3 and 8 copies per cell (FIG. 18A). These results demonstrated the high-level robustness of AP transposase-associated gene integration in the context of generating homogeneous stable cell pools.

Functionality and activity of the integrated transgenes was confirmed on the transcriptional level. Relative gene expression of the NRP1 transgene could be observed in all three experiments (FIG. 18B). Expression levels varied from 2-fold up to 9-fold elevated levels compared to a housekeeping gene. Again, the duplicates within each experiment displayed highly homogeneous gene expression levels. Across the experiments the homogeneity in gene expression was less prominent. This effect was to be expected, as gene transcription is highly influenced by downstream factors such as gene integration locus, epigenetic modifications and metabolic constraints.

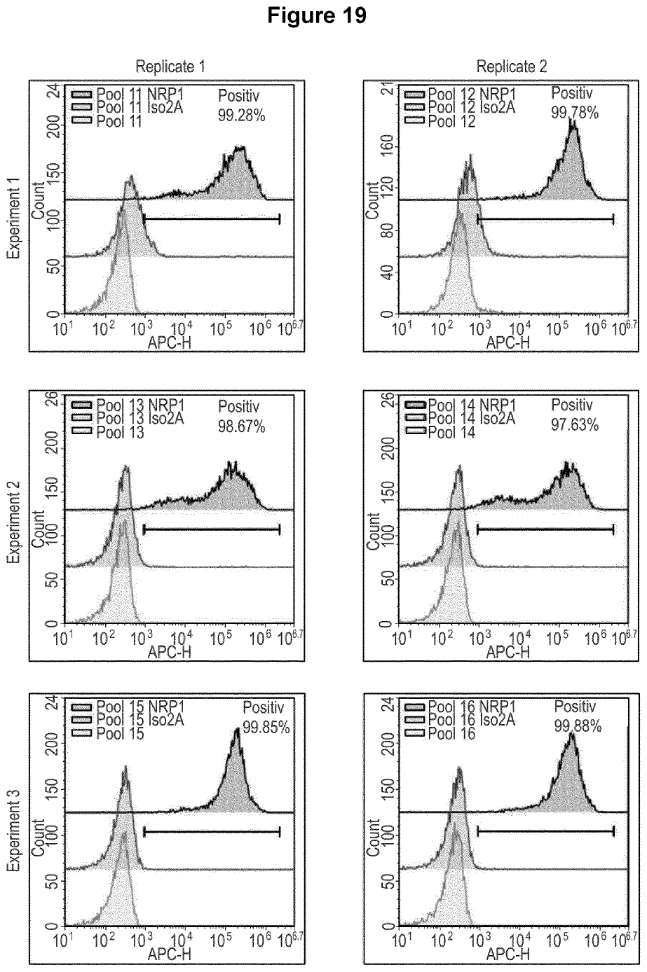

At the final stage of transgene expression the successful translation and localization of the NRP1 receptor was analyzed. Based on the action of the NRP1 receptor being translocated to the outer cell membrane a cell surface staining with a fluorochrome-labelled anti-NRP1 antibody was performed with the stably transfected cell pools. The stained cells were analyzed by flow cytometry to determine the signal intensity of fluorochrome-coupled antibody to the cell surface which correlates to NRP1 levels. To evaluate the effect of endogenous NRP1 levels an untransfected host cell sample was included in the analysis as a reference to determine the threshold for positive signals. Results of the cell surface staining experiments revealed high levels of NRP1 signals in all transfected cell pools with the majority of contained cell entities displaying positive signals (FIG. 19). Replicates within experiments were again highly comparable. Interestingly, samples from experiment 2 showed at least two cell populations being present in the cell pool with different signal intensity levels. This is coincided and could originate from the general lower relative gene expression levels observed in the previous analysis. In contrast, experiment 1 and 3 showed comparable signal intensities originating from obviously one major population. Overall, the histograms forthe lattertwo experiments are an indication for a highly homogeneous cell pool.

The stable cell pools expressing the cell surface receptor NRP1 generated with the new AP transposase system showed successful results on the genetic, transcriptional and translational level. The transfected cells produced adequate amounts of seemingly structural intact NRP1 receptors with a correct localization at the cell surface. All generated cell pools displayed high homologies in gene copy number and correlating results between transcription and translation rates. The AP transposase system proved its robustness and flexibility in generating stable pools expressing molecules beyond secreted proteins such as antibodies. Such cell pools can serve as source for generation of cell clones which can be utilized for various applications including cell-based bioassays.

### Example 12: Generation of Stable Cell Lines for Recombinant Protein Expression Other than Antibodies

In another approach we utilized the novel AP transposase system for the recombinant expression of alternative proteins like lipases. With that experiment, the ability of the AP transposase system to express tagged proteins other than monoclonal antibodies with different molecular properties was investigated.

Therefore, CHO-K1 cells were co-transfected with a transposon encoding for a lipase and the Providencia glutamine synthetase as well as a vector encoding for the AP transposase. After transfection, positively transfected cells were selected by cultivating the cell culture in glutamine free media. After the selection process, we aimed at capturing the overexpressed lipase and determine the yield of recombinant lipase by octet measurement. Overall, 5 lipases were overexpressed with 2 independent stable pools per lipase.

By using the previously described combination of the novel AP transposase system and the Providencia glutamine synthetase, recombinant pools expressing 5 different cHCPs were generated and could be scaled up into shake flasks within 7 to 10 days. Thereby, due to the efficient integration of the transposon a drop of only 15-40% viability was observed for the different transfections during the selection process. A negative control transfected only with the transposon was not recovering after selection start. Interestingly, the pools transfected with the same lipase showed strongly comparable viability curves over the selection phase (data not shown). Differences in selection time and viability between the recombinantly expressed lipases are likely correlating with influences of the overexpressed lipase on the CHO cell.

After the selection process was finished, a shake flask fed-batch was inoculated and cultivated for 8 days to recombinantly express the lipases with the AP transposase generated pools. For alle lipases significant amounts of lipase protein were expressed leading to protein yields after 8 days ranging from 30 mg/L up to 1.5 g/L depending on the overexpressed lipase and its influence on the CHO expression host (data not shown).

The expression of recombinant lipases using the Acyrthosiphon pisum transposase system was successful for all 5 lipases. Interestingly, the final lipase protein yields correlated strongly with the selection duration and the influence of the overexpressed lipase on CHO cell growth. Especially the overexpression of lipase 3 limited CHO cells in growth and was the least efficient lipase for recombinant overexpression. On the other side, after the overexpression of lipase 1, nearly no drop in viability was observed during pool selection and strong titers in the g/L range were obtained after just 8 days of fermentation. With these data, we were able to demonstrate the ability of the AP transposase system to recombinantly overexpress also proteins other than therapeutic mAbs if the overexpressed enzyme has not a harmful effect on the expression host itself.

Thus, the identified transposase/transposon pair derived from Acyrthosiphon pisum is a versatile system for generating stable cell lines expressing a variety of recombinant proteins in CHO cells and experiments are underway to confirm its applicability in human cells, such as HEK293 cells. It is postulated that the system can further be used for manufacturing of virus, particularly VSV and adeno-associated virus or virus-like particles derived thereof. It is further anticipated that the system can be used for stable integration in gene therapy, particularly ex vivo integration into autologous cells followed by adoptive cell transfer into a patient.

### Example 13: Related Homologs

A close relative to the transposase of Acyrthosiphon pisum was discovered, originating from Aphis craccivora (nucleic acid sequence: SEQ ID NO: 27; amino acid sequence: SEQ ID NO: 26). The amino acid sequences are highly homolog and share 98.3% of sequence identity of the transposase protein (Table 3).

TABLE 3
Sequence homology of both related transposase sequences

| | Aphis | Acyrthosiphon |
|---|---|---|
| | craccivora | pisum |
| | (Seq ID NO: 26) | (Seq ID NO: 4) |
| Aphis craccivora (Seq ID NO: 26 | | 98.3% |
| Acyrthosiphon pisum (Seq ID NO: 4) | 98.3% | |

When analyzing the proximal genomic sequence context, we discovered the ITR core sequences (SeqID 2) to be identical between both related systems. Moreover, the flanking sequences are highly homologous (Table 4 and Table 5).

TABLE 4
Sequence identity between left flanking sequences

| | Aphis | Acyrthosiphon |
|---|---|---|
| | craccivora | pisum |
| | (Seq ID NO: 29) | (Seq ID NO: 2) |
| Aphis craccivora (Seq ID NO: 29 | | 85.7% |
| Acyrthosiphon pisum (Seq ID NO: 2) | 85.7% | |

TABLE 5
Sequence identity between right flanking sequences

| | Aphis | Acyrthosiphon |
|---|---|---|
| | craccivora | pisum |
| | (Seq ID NO: 30) | (Seq ID NO: 3) |
| Aphis craccivora (Seq ID NO: 30 | | 95.8% |
| Acyrthosiphon pisum (Seq ID NO: 3) | 95.8% | |

Considering the high homology between transposase and ITR/flanking sequences for 15 Acyrthosiphon pisum and Aphis craccivora, the transposase/transposon combination of Aphis craccivora may further be functional. For this, Aphis creccivora transposase was generated comprising an N-terminal and a C-terminal NLS and an N-terminal Flag-tag (SEQ ID NO: 28).

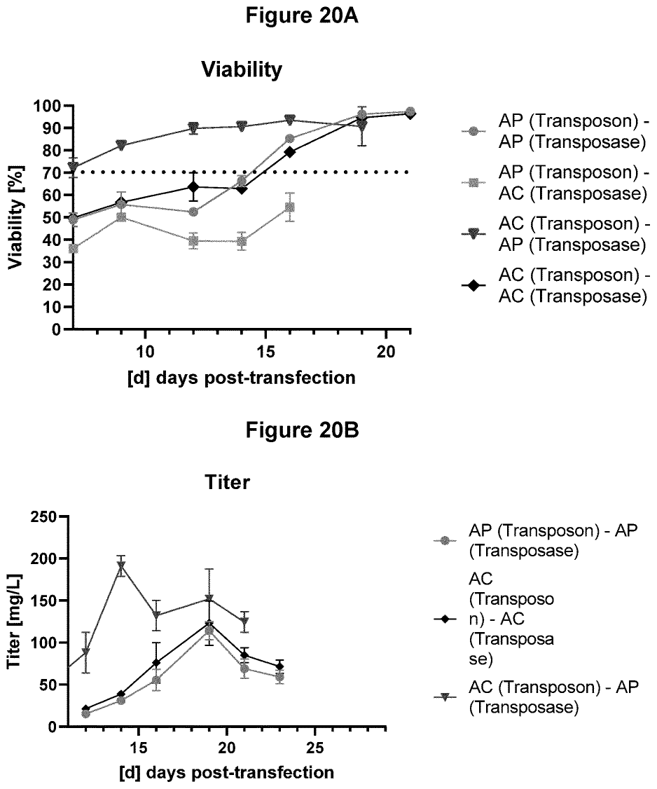

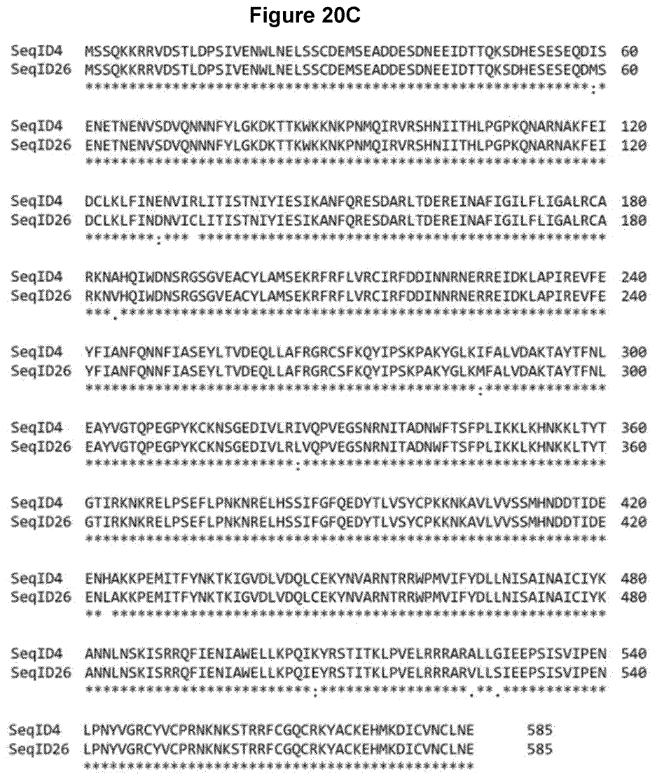

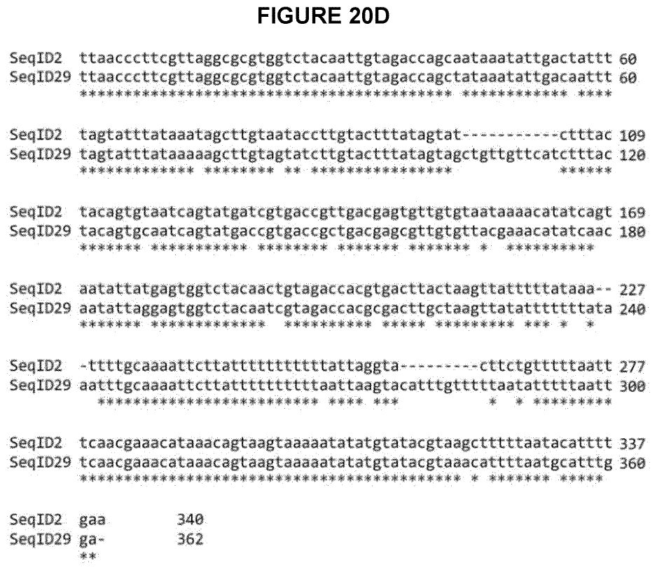

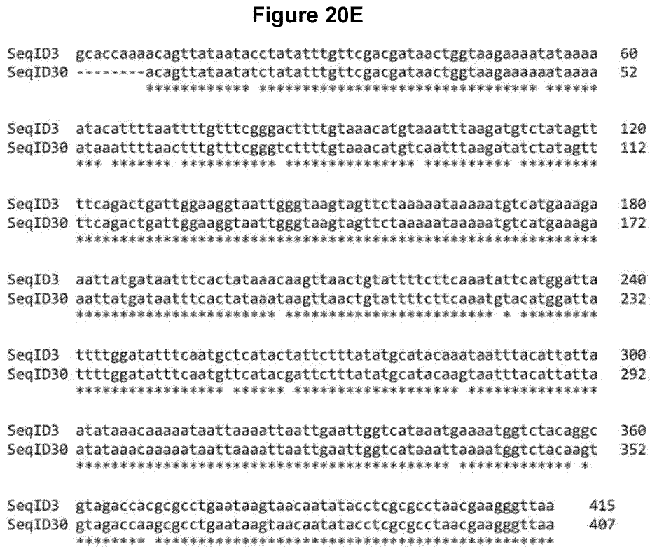

Driven by the high homology between transposase and ITR/flanking sequences for Acyrthosiphon pisum and Aphis craccivora, the transposase/transposon combination were tested for cross reactivity. Sequence alignments of AP transposase (SEQ ID NO: 4) and AC transposase (SEQ ID NO: 26) (FIG. 20C), left transposon flanking region of AP (SEQ ID NO: 2) and AC (SEQ ID NO: 29) (FIG. 20D) and right transposon flanking region of AP (SEQ ID NO: 3) and AC (SEQ ID NO: 30) (FIG. 20E) are shown in FIG. 20.

When transfecting glutamine synthetase deficient CHO cells with the Aphis craccivora transposase (SEQ ID NO: 26) and the Acyrthosiphon pisum-based transposon (harboring flanking sequences of SEQ ID NO: 2 and SEQ ID NO: 3 and a glutamine synthetase metabolic selection marker), the cells did not recover from selection (FIG. 20A), indicating that the Aphis craccivora transposase is not functional with Acyrthosiphon pisum-based transposons.

However, when the Aphis craccivora transposase (SEQ ID NO: 26) was combined with its natural flanking sequences (SEQ ID NO: 29 and SEQ ID NO: 30) CHO cells recovered from selection and yielded stable pools with high antibody expression titers which were comparable to the AP transposase/transposon pair, demonstrating the discovery of an additional functional highly related transposase/transposon in mammalian cells. Surprisingly, when combining the Aphis craccivora transposon haboring the flanking sequences of SEQ ID NO: 29 and SEQ ID NO: 30 with Acyrthosiphon pisum transposase (SEQ ID NO: 4), CHO cells recovered even significantly faster from selection (FIG. 20A) compared to either of their wildtype references. The combination led to a substantial increase in overall growth and productivity (FIG. 20B) as determined by antibody titer measurements, demonstrating cross-reactivity and an unexpected strong enhancement of the activity of the overall system. Since the AP and AC transposase only differ in 10 amino acids, mutational analysis will show which amino acid(s) are responsible for the slightly different activity of the two transposases.

Thus, both identified transposase/transposon pairs from Acyrthosiphon pisum and Aphis craccivora are excellent transposase/transposon pairs. Surprisingly, combining Aphis craccivora transposons with Acyrthosiphon pisum transposase was even more effective, although the inverse combination was not functional. Furthermore, the AP transposase and/or AC transposase may be further improved by using activity improved variants thereof as described herein, such as in Examples 9, 10 and 14.

### Example 14: Analysing AP Transposase Variants with Improved Activity Profile in Combination with AC Transposon

As shown in Example 13, when combining the Aphis craccivora (AC) transposon haboring the flanking sequences SEQ ID NO: 29 and SEQ ID NO: 30 with the AP transposase (SEQ ID NO: 4), CHO cells recovered significantly faster from selection compared to either of their wildtype controls (AP/AP or AC/AC). The combination further led to a substantial increase in overall growth and productivity. The superior performance of the AP Transposase+AC Transposon was confirmed in controlled bioreactors (Ambr15 system) and representative controlled process conditions. Furthermore, combinations of some of the AP transposase variants from Example 9 were further tested with the AC transposon and the AP transposon in parallel.

The cell culture performance was assessed in a two-step approach. Firstly, the selection duration was assessed, and the productivity was investigated in shaking flasks over multiple passages. These experiments confirmed the superior performance of the selected combination of mutations of the AP transposase over the wildtype AP enzyme and superiority of the AC transposon in combination with the AP transposase. Overall selection behavior was improved by up to 5 days, and the productivity levels were increased up to 3-fold (see Table 6).

TABLE 6
Overview of cell culture performance (selection
and productivity) in shaking flasks.

| | | Selection | MW Titer |
|---|---|---|---|
| | | Duration | (mg/L) |
| Transposase Variant | Transposon | (d) | passages |
| AP wildtype | AC | 7 | 98.4 |
| V212I + I215L + I363V + K365S | AC | 7 | 51.6 |
| K87Y + A264S + Q273V + L583F | AP | 9 | 98.9 |
| K87Y + A264S + Q273V + | AP | 9 | 92.2 |
| K576I + L583F + Del N584/E585 | | | |
| V212I + I215L + I363V + | AP | 9 | 91.2 |
| K365S + Q273V | | | |
| K87Y + V212I + I215L + | AP | 9 | 82.9 |
| I363V + K365S | | | |
| I363V + K365S + Q273V | AP | 9 | 64.9 |
| V212I + I215L + I363V + K365S | AP | 9 | 47 |
| K576I + L583F + Del N584 + | AP | 9 | 34.8 |
| E585 | | | |
| V212I + I215L + I363V + | AC | 9 | 110 |
| K365S + Q273V | | | |
| I363V + K365S + Q273V | AC | 9 | 61.6 |
| K87Y + V212I + I215L + | AC | 9 | 57.8 |
| I363V + K365S | | | |
| K87Y + A264S + Q273V + L583F | AC | 9 | 52 |
| K87Y + A264S + Q273V + | AC | 12 | 48.1 |
| K576I + L583F + Del N584/E585 | | | |
| AP wildtype | AP | 12 | 34 |

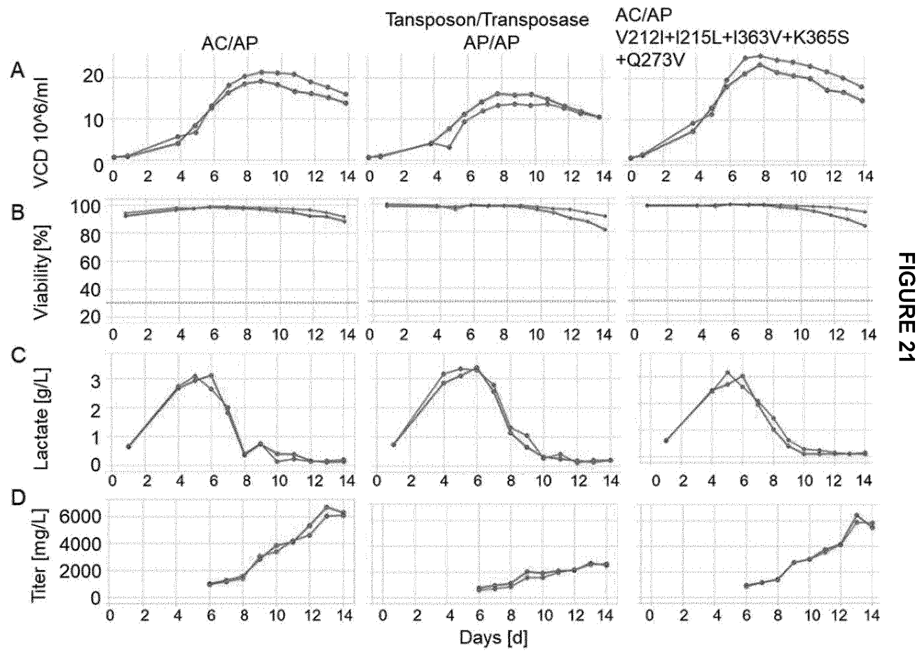

The same stable pools were subjected to a controlled bioprocess in an Ambr15 system to investigate the cell culture process conditions in more details. Besides cellular growth (viable cell density), the titer as well as important metabolic parameters were studied in a 14-day fed-batch production process (FIG. 21). The experiments in a controlled bioreactor confirmed the findings of the initial shaking flask study. The combination of AP wildtype transposase with the AC transposon as well as AP mutant V212I+I215L+I363V+K365S+Q273V with the AC or AP transposon showed superior performance, with titers reaching >6 g/L at day 14 for AC(transposon)-AP(transposase) and AC(transposon)-AP(transposase V212I+I215L+I363V+K365S+Q273V) sample pools. For comparison, the titers in AP(transposon)-AP(transposase) sample pools reached −3 g/L at day 14.

Thus, the data demonstrate that the performance of the transposase can be improved by introducing one or more activity improving mutations with positive effects on stable cell pool generation (selection behavior and productivity), which also translates into bioprocess parameters (cell viability and growth and productivity) in a controlled bioprocess of stable cell pools. Further, the data confirm the superiority of using AP transposase or AP transposase variants are particularly effective in combination with AC transposons.

### Example 15: Application of AP Transposase in a Human HEK293 Cell Line

To demonstrate the functionality of the AP transposase in human, suspension adapted HEK293F cells, 5×10⁶ cells were transfected in a 50 μL transfection volume with 5 μg transposon (using AP flanking regions, SEQ ID NOs: 2 and 3) encoding the fluorescent protein zsGreen and 4 μg AP transposase mRNA utilizing the MaxCyte® STx™ transfection device (MaxCyte, Rockville, MD, USA). The transfection was conducted using the HEK2 transfection program and R-50×8™ transfection slides (MaxCyte®) as described by the supplier. Control cells were transfected with 5 μg transposon without transposase mRNA. After transfection, cells were transferred into 40 mL fresh media and cultivated statically for 5 days at 37° C. with 5% CO₂ at 85% humidity. 5 days post-transfection, cells were transferred into shaking flasks at 37° C., 5% CO₂, 85% humidity and 95 rpm at 25 mm orbit and passaged every 3 to 4 days. During cultivation no selection for stable integrations was applied. 8 and 14 days post-transfection zsGreen expression was determined using the NovoCyte Flow Cytometer system (Agilent, Santa Clara, CA, USA). HEK293F cells maintain transfected plasmids for a few days, measurements at day 8 therefore represent expression from transient transfected plasmids, while measurements at day 14 represent expression following stable integration.

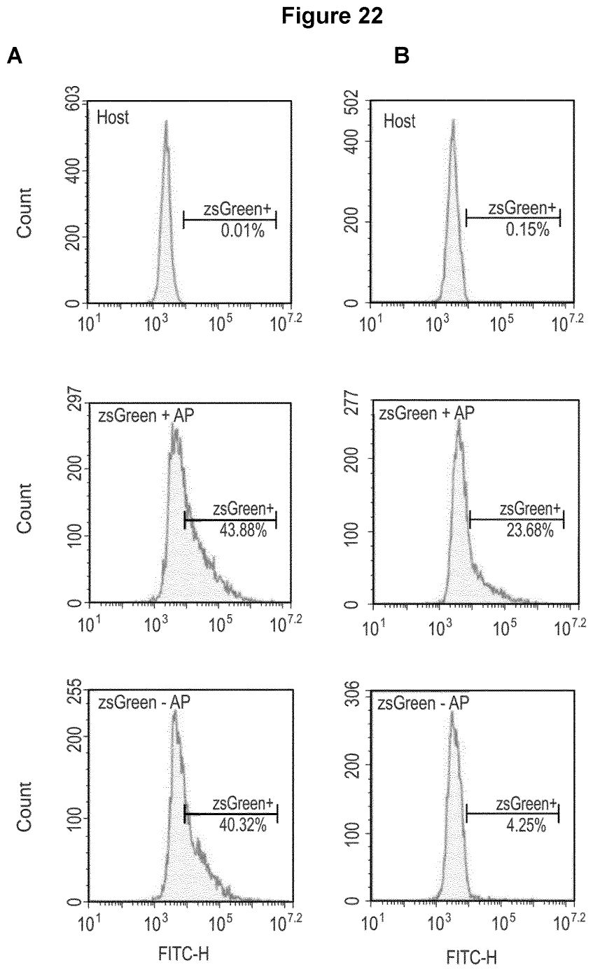

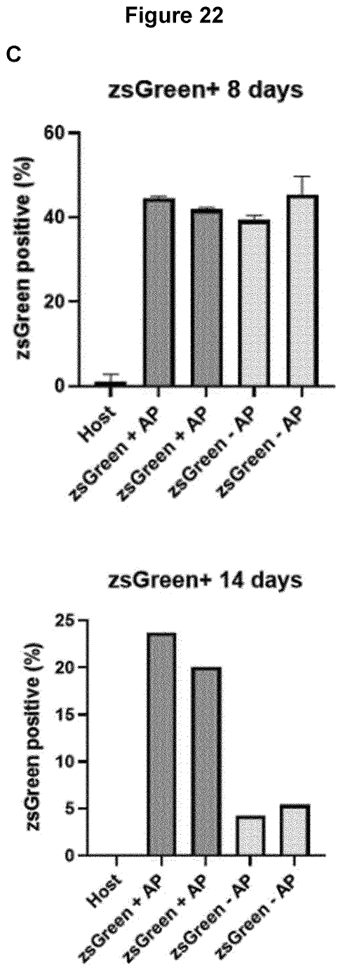

FIG. 22 (A and C) shows slightly above 40% zsGreen positive cells at day 8 in the presence and in the absence of AP transposase due to expression from transiently transfected plasmids. While % zsGreen expressing cells were neglectable 14 days post-transfection in the absence of AP transposase, about 23.68% of the cells remained zsGreen positive in the presence of AP transposase, demonstration stable integration (FIG. 22B and C). The data therefore demonstrate the compatibility of the AP transposase system with cells from species other than hamster.

### Example 16: Generation of Stable AAV HEK293 Production Cell Line Using AP Transposase

Generation of stable AAV producer cells: Stable adeno-associated virus (AAV) producer (packaging) cells comprising stably integrated rep and cap genes were established through a sequential transfection process involving two plasmids in adherent human embryonic kidney 293 (HEK293) cells (ATTC #CRL-1573).

The first plasmid (pBIG-540-AP/Cap) contained the AAV2 cap gene regulated by its endogenous p40 promoter, as well as a Neomycin resistance gene controlled by the simian virus 40 (SV40) promoter. These genes were flanked by the AP transposon flanking region sequences (SEQ ID NOs: 2 and 3).

The second plasmid harbored the AAV2 rep gene, in which one of its native promoters (p5) was substituted with an inducible tetracycline (Tet) promoter. Additionally, this plasmid encoded two proteins essential for Tet-promoter functionality, regulated by a mouse phosphoglycerate kinase (mpGK) promoter, and a hygromycin resistance gene controlled by the SV40 promoter. These sequences were also flanked by the AP transposon flanking region sequences (SEQ ID NOs: 2 and 3).

Both plasmids were sequentially transfected into the HEK293 cells alongside a plasmid encoding the AP transposase gene with additional N- and C-terminal NLS (SEQ ID NO: 18). Initially, HEK293 cells were transfected with pBIG-540-AP/Cap by electroporation using a Neon transfection system (Thermo Fisher Scientific, Waltham, Massachusetts, USA). 24 hours post-transfection, cells underwent antibiotic selection via treatment with 250 μg/pL Geneticin G418. After approximately four weeks of selection and cellular regeneration, the second plasmid (pBIG-560-AP/TET-Rep) was transfected into the stable recombinant cap-carrying HEK293 cell pools also by electroporation. Cells were subjected to a second round of antibiotic selection using both 250 μg/μL Geneticin G418 and 50 μg/μL Hygromycin at 24 hours post-transfection. Upon cell recovery from double antibiotic selection (approximately 3-4 weeks), stable Rep/Cap-carrying HEK293 cell pools (HEK293_Cap_Tet-Rep cells) were employed for AAV production.

AAV production in unmodified HEK293 as well as stable HEK293_Cap_Tet-Rep producer cells: A total of 7×10⁵ HEK293 or HEK293_Cap_Tet-Rep cells were seeded into 6-well plates using Dulbecco's Modified Eagle Medium (DMEM) (Thermo Fisher Scientific, Waltham, Massachusetts, USA) supplemented with 10% fetal bovine serum (FBS). After 24 hours, cells were transfected with plasmids from the AAV-MAX Control Plasmid Kit (Thermo Fisher Scientific, Waltham, Massachusetts, USA, #A47672). For the transfection, plasmids pHelper and pAAV-CMV-EmGFP (pTransfer) were combined at a 1:1 molar ratio (totaling 1.4 μg DNA per well) and complexed for transfection with FectoVIR (4.2 μL per well; Polyplus Transfection SA, Illkirch-Graffenstaden, France). During AAV production, the medium was supplemented with 500 ng/mL of doxycycline to activate the Tet-inducible promoter. At 72 hours post-transfection, cells were detached using a cell scraper. Both cells and cell culture supernatant were transferred to tubes and centrifuged at 300×g for 7 minutes. The cell culture supernatant was then transferred to a new tube, while the cell pellet was resuspended in 300 μL of phosphate-buffered saline (PBS). Both tubes were stored at −80° C. for subsequent analysis.

Transduction of HEK293 cell with AAV samples: 1*10⁴ HEK293 cells were seeded into 96-well microplates. 24 hours later, cell culture medium was removed and cells were treated with 0.5 μg Mytomycin C (in DMEM medium) for 1 hour. Subsequently, cells were washed with PBS and 100 μL of fresh DMEM medium was provided. For transduction, 50 μL of AAV sample (either different dilutions of AAV-containing lysate or non-diluted AAV-containing cell culture supernatant) were transferred to the adherently grown HEK293 cells per well. After 48 hours of incubation, emerald green fluorescent protein (emGFP) fluorescence was analyzed by fluorescence microscopy (using a Cytation 5 imaging system).

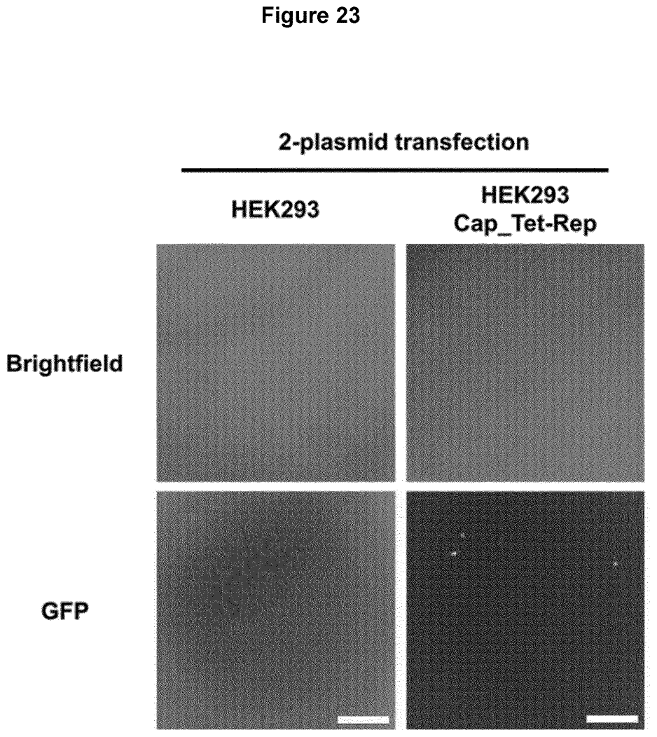

While no AAV-mediated emGFP expression was observed in cells treated with culture supernatant derived from HEK293 transfected with pHelper and pTransfer plasmids (which lack Rep and Cap gene expression), emGFP expression was detected in cells treated with the cell culture supernatant derived from stable HEK293_Cap_Tet-Rep cells indicating that the AAV Rep and Cap genes were successfully stably integrated into HEK293 cells using the AP transposase in combination with AP transposons (FIG. 23).

The data demonstrate that stable AAV production cell lines based on human HEK293 cells could be generated by stable integration of the required AAV genes via AP transposase. This extends the applicability of the AP transposase system far beyond the classical protein production and to a different cell type.

### Example 17: Activity Testing of Putative Transposase/Transposon-Like Sequences

As described in Example 1, 384 potential different transposase/transposon-like sequences were identified based on published genomic sequences based on characteristic motifs, such as complete transposase open reading frame, catalytic DDE/DDD motifs and ITR flanking sequences. 14 transposase/transposon-like sequences were experimentally tested and activity of the transposase was demonstrated only for AP transposase with its respective transposon sequences. Transfection experiments using circular plasmid DNA vectors and mRNA coding for the respective transposase were performed as described in Example 2 for the AP transposease. Results from selected transposases and their selected transposon flanking sequences as specified in Table 7 below are shown in FIG. 24.

TABLE 7
Selected transposase/transposon-like sequences in
addition to the AP transposase/transposon pair

| | amino acid | | |
|---|---|---|---|
| | sequence | sequence left | Sequence right |
| Species | Transposase | flanking region | flanking region |
| Alypsia | SEQ ID NO: 71 | SEQ ID NO: 72 | SEQ ID NO: 73 |
| californica | | | |
| Onthophagus | SEQ ID NO: 74 | SEQ ID NO: 75 | SEQ ID NO: 76 |
| taurus | | | |
| Onthophagus | SEQ ID NO: 77 | SEQ ID NO: 78 | SEQ ID NO: 79 |
| taurus | | | |
| Onthophagus | SEQ ID NO: 80 | SEQ ID NO: 81 | SEQ ID NO: 82 |
| taurus | | | |
| Vanessa | SEQ ID NO: 83 | SEQ ID NO: 84 | SEQ ID NO: 85 |
| tameamea | | | |
| Acyrthosiphon | SEQ ID NO: 4 | SEQ ID NO: 2 | SEQ ID NO: 3 |
| pisum | | | |

The results demonstrate that unexpectedly a single transposease/DNA transposon pair was found to show transposition activity. While prediction based on genetic elements in database sequences allows the identification of numerous transposase/transposon-like sequences, sequence-based prediction on the activity and functionality of these sequences is impossible and requires experimental testing without any expectation that a functional pair is within the tested sequences. The identification of the active A. pisum transposase/DNA transposon pair was therefore surprising.

Sequence listing

| SEQ | |
|---|---|
| ID | |
| NO: | description |
| 1 | Inverted terminal repeat (ITR): CCCTTCGTTAGG |
| 2 | AP left transposon flanking region (340 nt) |
| 3 | AP right transposon flanking region (415 nt) |
| 4 | AP Transposase wildtype (amino acid sequence) |
| 5 | Heterologous nuclear localization signal: PKKKRKV |
| 6 | AP Transposase wildtype with N-terminal heterologous NLS (amino acid sequence) |
| 7 | AP Transposase wildtype with N- and C-terminal heterologous NLS and N-Terminal |
| | Flag-tag (amino acid sequence) |
| 8 | Truncated AP left transposon flanking region (297 nt): |
| | TTAACCCTTCGTTAGGCGCGTGGTCTACAATTGTAGACCAGCAATAAATATTGACT |
| | ATTTTAGTATTTATAAATAGCTTGTAATACCTTGTACTTTATAGTATCTTTACTACAG |
| | TGTAATCAGTATGATCGTGACCGTTGACGAGTGTTGTGTAATAAAACATATCAGTA |
| | ATATTATGAGTGGTCTACAACTGTAGACCACGTGACTTACTAAGTTATTTTTATAAA |
| | TTTTGCAAAATTCTTATTTTTTTTTTTATTAGGTACTTCTGTTTTTAATTTCAACGAAA |
| | CATAAACAGTA |
| 9 | Truncated AP right transposon flanking region (260 nt): |
| | CTAAAAATAAAAATGTCATGAAAGAAATTATGATAATTTCACTATAAACAAGTTAAC |
| | TGTATTTTCTTCAAATATTCATGGATTATTTTGGATATTTCAATGCTCATACTATTCT |
| | TTATATGCATACAAATAATTTACATTATTAATATAAACAAAAATAATTAAAATTAATTG |
| | AATTGGTCATAAATGAAAATGGTCTACAGGCGTAGACCACGCGCCTGAATAAGTA |
| | ACAATATACCTCGCGCCTAACGAAGGGTTAA |
| 10 | Truncated AP left transposon flanking region (214 nt): |
| | TTAACCCTTCGTTAGGCGCGTGGTCTACAATTGTAGACCAGCAATAAATATTGACT |
| | ATTTTAGTATTTATAAATAGCTTGTAATACCTTGTACTTTATAGTATCTTTACTACAG |
| | TGTAATCAGTATGATCGTGACCGTTGACGAGTGTTGTGTAATAAAACATATCAGTA |
| | ATATTATGAGTGGTCTACAACTGTAGACCACGTGACTTACTAAG |
| 11 | Truncated AP right transposon flanking region (154 nt): |
| | TACTATTCTTTATATGCATACAAATAATTTACATTATTAATATAAACAAAAATAATTAA |
| | AATTAATTGAATTGGTCATAAATGAAAATGGTCTACAGGCGTAGACCACGCGCCTG |
| | AATAAGTAACAATATACCTCGCGCCTAACGAAGGGTTAA |
| 12 | Truncated AP left transposon flanking region (86 nt): |
| | TTAACCCTTCGTTAGGCGCGTGGTCTACAATTGTAGACCAGCAATAAATATTGACT |
| | ATTTTAGTATTTATAAATAGCTTGTAATAC |
| 13 | Truncated AP right transposon flanking region (57 nt): |
| | GCGTAGACCACGCGCCTGAATAAGTAACAATATACCTCGCGCCTAACGAAGGGTT |
| | AA |
| 14 | Heterologous nuclear localization signal: KRPAATKKAGQAKKKK |
| 15 | Reverse complementary ITR: CCTAACGAAGGG |
| 16 | AP Transposase wildtype (nucleotide sequence) |
| 17 | AP Transposase wildtype with N-terminal heterologous NLS (nucleotide sequence) |
| 18 | AP Transposase wildtype with N- and C-terminal heterologous NLS and N-Terminal |
| | Flag-tag (nucleotide sequence) |
| 19 | Nuclear localization signal (nucleotide sequence) (21 nt): cctaagaagaagcgcaaagtc |
| 20 | Nuclear localization signal (nucleotide sequence) (48 nt): |
| | aaacgccctgccgctacgaagaaagctgggcaggccaagaagaagaag |
| 21 | Heterologous nuclear localization signal (nucleotide sequence) (45 nt): |
| | ccaaagaaaaaacgaaaggtggggattcacggcgttcctgcagct |
| 22 | Heterologous nuclear localization signal (amino acid sequence) with linker sequence: |
| | PKKKRKVGIHGVPAA |
| 23 | Flag-tag (amino acid sequence): DYKDHDGDYKDHDIDYKDDDDK |
| 24 | Flag-tag (nucleotide sequence) |
| 25 | Nuclear localization signal (amino acid sequence): QKKRRV |
| 26 | Aphis craccivora (AC) transposase, wild type (amino acid sequence) |
| 27 | AC transposase, wild type (nucleotide sequence) |
| 28 | AC transposase wildtype with N- and C-terminal heterologous NLS and N-terminal Flag- |
| | tag (nucleotide sequence) |
| 29 | AC left transposon flanking region (nucleotide sequence) (362 nt): |
| | ttaacccttcgttaggcgcgtggtctacaattgtagaccagctataaatattgacaattttagtatttataaaaagcttgtagtat |
| | cttgtactttatagtagctgttgttcatctttactacagtgcaatcagtatgaccgtgaccgctgacgagcgttgtgttacgaaa |
| | catatcaacaatattaggagtggtctacaatcgtagaccacgcgacttgctaagttatatttttttataaatttgcaaaattctta |
| | ttttttttttaattaagtacatttgtttttaatatttttaatttcaacgaaacataaacagtaagtaaaaatatatgtatacgtaaac |
| | attttaatgcatttgga |
| 30 | AC right transposon flanking region (nucleotide sequence) (407 nt): |
| | acagttataatatctatatttgttcgacgataactggtaagaaaaaataaaaataaattttaactttgtttcgggtcttttgtaaa |
| | catgtcaatttaagatatctatagttttcagactgattggaaggtaattgggtaagtagttctaaaaataaaaatgtcatgaaa |
| | gaaattatgataatttcactataaataagttaactgtattttcttcaaatgtacatggattattttggatatttcaatgttcatacga |
| | ttctttatatgcatacaagtaatttacattattaatataaacaaaaataattaaaattaattgaattggtcataaattaaaatggtc |
| | tacaagtgtagaccaagcgcctgaataagtaacaatatacctcgcgcctaacgaagggttaa |
| 31 | AP transposase wildtype with N- and C-terminal heterologous NLS (nucleotide |
| | sequence) |
| 32 | AP transposase wildtype with N- and C-terminal heterologous NLS (amino acid |
| | sequence) |
| 33 | AP K87Y variant transposase wildtype with N- and C-terminal heterologous NLS |
| | (nucleotide sequence) |
| 34 | AP K87Y variant Transposase with N- and C-terminal heterologous NLS (amino acid |
| | sequence) |
| 35 | AP 1363V and K365S variant transposase with N- and C-terminal heterologous NLS |
| | (amino acid sequence) |
| 36 | AP A264S and Q273V variant transposase with N- and C-terminal heterologous NLS |
| | (amino acid sequence) |
| 37 | AP K87Y and A264S variant transposase with N- and C-terminal heterologous NLS |
| | (amino acid sequence) |
| 38 | Target sequence and inverted terminal repeat (ITR): TTAACCCTTCGTTAGG |
| 39 | reverse complementary ITR and target sequence: CCTAACGAAGGGTTAA |
| 40 | Target sequence and inverted terminal repeat (ITR) and binding motif: |
| | TTAACCCTTCGTTAGGCGCG |
| 41 | Binding motif and reverse complementary ITR and target: |
| | sequence:CGCGCCTAACGAAGGGTTAA |
| 42 | Internal repeat motif: TGGTCTAC |
| 43 | Inverted internal repeat (IIR) motif: TGGTCTACannnGTAGACCA |
| 44 | Inverted internal repeat (IIR) motif: TGGTCTACarbyGTAGACCA |
| 45 | Left arm target sequence, ITR and IIR: |
| | TTAACCCTTCGTTAGGCGCGTGGTCTACAnnnGTAGACCA |
| 46 | Left arm target sequence, ITR and IIR: |
| | TTAACCCTTCGTTAGGCGCGTGGTCTACAATTGTAGACCA |
| 47 | Truncated generic right transposon flanking region (67 nt): |
| | TGGTCTACAnnnGTAGACCAmGCGCCTGAATAAGTAACAATATACCTCGCGCCTAA |
| | CGAAGGGTTAA |
| 48 | Truncated AP right transposon flanking region (67 nt): |
| | TGGTCTACAnnnGTAGACCACGCGCCTGAATAAGTAACAATATACCTCGCGCCTAA |
| | CGAAGGGTTAA |
| 49 | Truncated AP right transposon flanking region (67 nt): |
| | TGGTCTACAGGCGTAGACCACGCGCCTGAATAAGTAACAATATACCTCGCGCCTA |
| | ACGAAGGGTTAA |
| 50 | Binding motif: AGGCGCG |
| 51 | Target sequence: TTAA |
| 52 | NLS linker sequence: GIHGVPAA |
| 53 | Modified truncated AP transposon, left arm (with additional binding site-binding site |
| | IIR motif of right arm (reverse SEQ ID NO: 54) highlighted in bold): |
| | TTAACCCTTCGTTAGGCGCGTGGTCTACAATTGTAGACCAAGGCGCGAGGTATAT |
| | TGTTACTTATTCAGGCGCGTGGTCTACGCCTGTAGACCAG |
| 54 | Binding site-binding site-IIR motif of AP transposon right arm (nt 349-402 of SEQ ID NO: |
| | 3): |
| | TGGTCTACAGGCGTAGACCACGCGCCTGAATAAGTAACAATATACCTCGCGCCT |
| 55 | Modified AP transposon, left arm (with additional binding site-binding site-IIR motif |
| | of right arm (reverse SEQ ID NO: 54) and further binding motif highlighted in bold): |
| | TTAACCCTTCGTTAGGCGCGTGGTCTACAATTGTAGACCAAGGCGCGAGGTATAT |
| | TGTTACTTATTCAGGCGCGTGGTCTACGCCTGTAGACCAGCAATAAATATTGACT |
| | ATTTTAGTATTTATAAATAGCTTGTAATACCTTGTACTTTATAGTATCTTTACTACAG |
| | TGTAATCAGTATGATCGTGACCGTTGACGAGTGTTGTGTAATAAAACATATCAGTA |
| | ATATTATGAGTGGTCTACAACTGTAGACCACGTGACTTACTAAGTTATTTTTATAAA |
| | TTTTGCAAAATTCTTATTTTTTTTTTTATTAGGTACTTCTGTTTTTAATTTCAACGAAA |
| | CATAAACAGTAAGTAAAAATATATGTATACGTAAGCTTTTTAATACATTTTGAA |
| 56 | Modified AP transposon, right arm (with additional binding site-binding site-IIR motif |
| | of right arm highlighted in bold): |
| | GCACCAAAACAGTTATAATACCTATATTTGTTCGACGATAACTGGTAAGAAAATATA |
| | AAAATACATTTTAATTTTGTTTCGGGACTTTTGTAAACATGTAAATTTAAGATGTCTA |
| | TAGTTTTCAGACTGATTGGAAGGTAATTGGGTAAGTAGTTCTAAAAATAAAAATGT |
| | CATGAAAGAAATTATGATAATTTCACTATAAACAAGTTAACTGTATTTTCTTCAAATA |
| | TTCATGGATTATTTTGGATATTTCAATGCTCATACTATTCTTTATATGCATACAAATA |
| | ATTTACATTATTAATATAAACAAAAATAATTAAAATTAATTGAATTGGTCATAAATGA |
| | AAATGGTCTACAGGCGTAGACCACGCGCCTGAATAAGTAACAATATACCTCGCG |
| | CCTTGGTCTACAGGCGTAGACCACGCGCCTGAATAAGTAACAATATACCTCGCGC |
| | CTAACGAAGGGTTAA |
| 57 | Modified AP transposon, left arm (with additional binding site-binding site-IIR motif |
| | of right arm (reverse SEQ ID NO: 54) and further binding motif highlighted in bold): |
| | TTAACCCTTCGTTAGGCGCGTGGTCTACAATTGTAGACCAAGGCGCGAGGTATAT |
| | TGTTACTTATTCAGGCGCGTGGTCTACGCCTGTAGACCAAGGCGCGGCAATAAA |
| | TATTGACTATTTTAGTATTTATAAATAGCTTGTAATACCTTGTACTTTATAGTATCTT |
| | TACTACAGTGTAATCAGTATGATCGTGACCGTTGACGAGTGTTGTGTAATAAAACA |
| | TATCAGTAATATTATGAGTGGTCTACAACTGTAGACCACGTGACTTACTAAGTTATT |
| | TTTATAAATTTTGCAAAATTCTTATTTTTTTTTTTATTAGGTACTTCTGTTTTTAATTT |
| | CAACGAAACATAAACAGTAAGTAAAAATATATGTATACGTAAGCTTTTTAATACATT |
| | TTGAA |
| 58 | Truncated AP left transposon flanking region (200 nt): |
| | TTAACCCTTCGTTAGGCGCGTGGTCTACAATTGTAGACCAGCAATAAATATTGACT |
| | ATTTTAGTATTTATAAATAGCTTGTAATACCTTGTACTTTATAGTATCTTTACTACAG |
| | TGTAATCAGTATGATCGTGACCGTTGACGAGTGTTGTGTAATAAAACATATCAGTA |
| | ATATTATGAGTGGTCTACAACTGTAGACCA |
| 59 | Truncated AP right transposon flanking region (77 nt): |
| | TAAATGAAAATGGTCTACAGGCGTAGACCACGCGCCTGAATAAGTAACAATATAC |
| | CTCGCGCCTAACGAAGGGTTAA |
| 60 | Truncated AC right transposon flanking region (67 nt): |
| | TGGTCTACAnnnGTAGACCAAGCGCCTGAATAAGTAACAATATACCTCGCGCCTAA |
| | CGAAGGGTTAA |
| 61 | Truncated AC right transposon flanking region (67 nt): |
| | TGGTCTACAAGTGTAGACCAAGCGCCTGAATAAGTAACAATATACCTCGCGCCTA |
| | ACGAAGGGTTAA |
| 62 | Truncated AC left transposon flanking region (nucleotide sequence) (320 nt): |
| | ttaacccttcgttaggcgcgtggtctacaattgtagaccagctataaatattgacaattttagtatttataaaaagcttgtagtat |
| | cttgtactttatagtagctgttgttcatctttactacagtgcaatcagtatgaccgtgaccgctgacgagcgttgtgttacgaaa |
| | catatcaacaatattaggagtggtctacaatcgtagaccacgcgacttgctaagttatatttttttataaatttgcaaaattctta |
| | ttttttttttaattaagtacatttgtttttaatatttttaatttcaacgaaacataaacagta |
| 63 | Truncated AC left transposon flanking region (nucleotide sequence) (225 nt): |
| | ttaacccttcgttaggcgcgtggtctacaattgtagaccagctataaatattgacaattttagtatttataaaaagcttgtagtat |
| | cttgtactttatagtagctgttgttcatctttactacagtgcaatcagtatgaccgtgaccgctgacgagcgttgtgttacgaaa |
| | catatcaacaatattaggagtggtctacaatcgtagaccacgcgacttgctaag |
| 64 | Truncated AC left transposon flanking region (nucleotide sequence) (211 nt): |
| | ttaacccttcgttaggcgcgtggtctacaattgtagaccagctataaatattgacaattttagtatttataaaaagcttgtagtat |
| | cttgtactttatagtagctgttgttcatctttactacagtgcaatcagtatgaccgtgaccgctgacgagcgttgtgttacgaaa |
| | catatcaacaatattaggagtggtctacaatcgtagacca |
| 65 | Truncated AC right transposon flanking region (nucleotide sequence) (260 nt): |
| | ctaaaaataaaaatgtcatgaaagaaattatgataatttcactataaataagttaactgtattttcttcaaatgtacatggatta |
| | ttttggatatttcaatgttcatacgattctttatatgcatacaagtaatttacattattaatataaacaaaaataattaaaattaatt |
| | gaattggtcataaattaaaatggtctacaagtgtagaccaagcgcctgaataagtaacaatatacctcgcgcctaacgaa |
| | gggttaa |
| 66 | Truncated AC right transposon flanking region (nucleotide sequence) (154 nt): |
| | tacgattctttatatgcatacaagtaatttacattattaatataaacaaaaataattaaaattaattgaattggtcataaattaaa |
| | atggtctacaagtgtagaccaagcgcctgaataagtaacaatatacctcgcgcctaacgaagggttaa |
| 67 | AC right transposon flanking region (nucleotide sequence) (77 nt): |
| | taaattaaaatggtctacaagtgtagaccaagcgcctgaataagtaacaatatacctcgcgcctaacgaagggttaa |
| 68 | IIR motif and binding motif (left arm): TGGTCTACAnnnGTAGACCAmGCGCCT |
| 69 | IIR motif and binding motif (left arm): TGGTCTACAnnnGTAGACCACGCGCCT |
| 70 | IIR motif and binding motif (left arm): TGGTCTACAnnnGTAGACCAAGCGCCT |
| 71 | Alypsia californica transposase (amino acid sequence) |
| 72 | Alypsia californica left flanking region (nucleotide sequence) |
| 73 | Alypsia californica right flanking region (nucleotide sequence) |
| 74 | Onthophagus taurus transposase (amino acid sequence) |
| 75 | Onthophagus taurus left flanking region (nucleotide sequence) |
| 76 | Onthophagus taurus right flanking region (nucleotide sequence) |
| 77 | Onthophagus taurus transposase (amino acid sequence) |
| 78 | Onthophagus taurus left flanking region (nucleotide sequence) |
| 79 | Onthophagus taurus right flanking region (nucleotide sequence) |
| 80 | Onthophagus taurus transposase (amino acid sequence) |
| 81 | Onthophagus taurus left flanking region (nucleotide sequence) |
| 82 | Onthophagus taurus right flanking region (nucleotide sequence) |
| 83 | Vanessa tameamea transposase (amino acid sequence) |
| 84 | Vanessa tameamea left flanking region (nucleotide sequence) |
| 85 | Vanessa tameamea right flanking region (nucleotide sequence) |

Nucleotide symbols: n = a or c or g or t/u, m = a or c, r = a or g, b = c, g or t/u, y = c or t/u according to the Table 1 of Section 1 of WIPO Standard 26 (Date: November 2021)

1. A DNA transposon comprising a heterologous polynucleotide flanked by transposon flanking regions, wherein one transposon flanking region comprises the nucleotide sequence of SEQ ID NO: 38 at one transposon end; and the other transposon flanking region comprises the nucleotide sequence of SEQ ID NO: 39 at the other transposon end.

2. The DNA transposon of claim 1, wherein
(a) the one transposon flanking region comprises the nucleotide sequence of SEQ ID NO: 40 at one transposon end; and the other transposon flanking region comprises the nucleotide sequence of SEQ ID NO: 41 at the other transposon end;
(b) the one and the other transposon flanking region each further comprises at least one inverted internal repeat (IIR) comprising (i) an internal repeat motif comprising the nucleotide sequence tggtctac and (ii) its reverse complementary sequence (iii) separated by six to three; and/or
(c) (i) the one transposon flanking region comprises the nucleotide sequence of SEQ ID NO: 45 or 46 and further an inverted internal repeat motif having the nucleotide sequence of SEQ ID NO: 43 separated by about 50 to 200 nucleotides; and/or the other transposon flanking region comprises the nucleotide sequence of SEQ ID NO: 47 or a nucleotide sequence having at least 95% sequence identity with SEQ ID NO: 47, wherein the sequence comprises at least the nucleotide sequence of SEQ ID NO: 41 and the nucleotide sequence of SEQ ID NO: 43; or (ii) wherein the one and the other transposon flanking regions are inverted and comprise reverse complementary sequences of the sequences recited in (i).

3. The DNA transposon of claim 1, wherein
(a) (i) the one transposon flanking region comprises the nucleotide sequence of SEQ ID NO: 45 or 46 and further an inverted internal repeat motif having the nucleotide sequence of SEQ ID NO: 43 separated by about 50 to 200 nucleotides and wherein the one transposon flanking region comprises a nucleotide sequence having 85% sequence identity with SEQ ID NO: 10 or 63, or with SEQ ID NO: 2 or 29; and/or the other transposon flanking region comprises the nucleotide sequence of SEQ ID NO: 47 or a nucleotide sequence having at least 95% sequence identity with SEQ ID NO: 47, wherein the sequence comprises at least the nucleotide sequence of SEQ ID NO: 41 and the nucleotide sequence of SEQ ID NO: 43; or (ii) wherein the one and the other transposon flanking regions are inverted and comprise reverse complementary sequences of the sequences recited in (i); and/or
(b) (i) the one transposon flanking region comprises a nucleotide sequence having at least 85% nucleotide sequence identity with SEQ ID NO: 2, 8, 10, or 58 and/or the other transposon flanking region comprises a nucleotide sequence having at least 90% nucleotide sequence identity with SEQ ID NO: 3, 9, 11 or 59; or (ii) wherein the one and the other transposon flanking regions are inverted and comprise reverse complementary sequences of the sequences recited in (i); and/or
(c) (i) the one transposon flanking region comprises the nucleotide sequence of SEQ ID NO: 2, 8, 10 or 58 or the sequence of SEQ ID NO: 29, 62, 63 or 64 and/or the other transposon flanking region comprises the nucleotide sequence of SEQ ID NO: 3, 9, 11, 59, 48 or 49 or the nucleotide sequence of SEQ ID NO: 30, 65, 66, 67, 60 or 61; or (ii) wherein the one and the other transposon flanking regions are inverted and comprise reverse complementary sequences of the sequences recited in (i); or
(d) (i) the one transposon flanking region comprises a nucleotide sequence having at least 95% sequence identity with SEQ ID NO: 29, 62, 63 or 64 and/or the other transposon flanking region comprises a nucleotide sequence having at least 97% sequence identity with SEQ ID NO: 30, 65, 66, 67, 60 or 61; or (ii) wherein the one and the other transposon flanking regions are inverted and comprise reverse complementary sequences of the sequences recited in (i).

4. The DNA transposon of claim 1, wherein the heterologous polynucleotide comprises at least one sequence selected from the group consisting of a sequence encoding a gene of interest, a complementary DNA (cDNA), a genome of interest and another genetic element.

5. The DNA transposon of claim 1, wherein the transposon is transposable by a transposase comprising the amino acid sequence of SEQ ID NO: 4.

6. An expression vector comprising the DNA transposon of claim 1.

7. A recombinant transposase comprising:
(i) an amino acid sequence having at least 90% sequence identity with amino acids 10 to 585 of SEQ ID NO: 4 and at least one heterologous nuclear localization signal (NLS) fused to the transposase; or
(ii) an amino acid sequence having at least 90% sequence identity with amino acids 10 to 585 of SEQ ID NO: 4, wherein the transposase comprises at least one mutation.

8. The recombinant transposase of claim 7, wherein the recombinant transposase comprises an amino acid sequence having at least 90% sequence identity with amino acids 10 to 585 of SEQ ID NO: 4, wherein the transposase comprises at least one mutation.

9. A polynucleotide encoding a transposase comprising an amino acid sequence having at least 90% sequence identity with amino acids 10 to 585 of SEQ ID NO: 4 operably linked to a eukaryotic promoter.

10. A polynucleotide encoding the recombinant transposase of claim 7.

11. An expression vector, wherein the expression vector:
(i) comprises a polynucleotide encoding the recombinant transposase of claim 7; or
(ii) encodes the recombinant transposase of claim 7.

12. An isolated mRNA encoding the recombinant transposase of claim 7.

13. An expression system or a kit comprising
(a) a recombinant transposase source selected from the group consisting of
(i) an expression vector, wherein the expression vector (a) comprises a polynucleotide encoding the recombinant transposase of claim 7 or (b) encodes the recombinant transposase of claim 7;
(ii) an isolated mRNA encoding the recombinant transposase of claim 7; and
(iii) the recombinant transposase of claim 7; and
(b) a DNA transposon comprising a heterologous polynucleotide flanked by transposon flanking regions, wherein one transposon flanking region comprises the nucleotide sequence of SEQ ID NO: 38 at one transposon end; and the other transposon flanking region comprises the nucleotide sequence of SEQ ID NO: 39 at the other transposon end; or an expression vector comprising said DNA transposon.

14. A eukaryotic cell comprising the DNA transposon of claim 1, wherein the eukaryotic cell is a yeast or a mammalian cell.

15. A method for preparing a cell comprising a stably integrated heterologous polynucleotide, comprising
(a) introducing into a eukaryotic cell a DNA molecule comprising (i) a DNA transposon comprising a heterologous polynucleotide flanked by transposon flanking regions, wherein one transposon flanking region comprises the nucleotide sequence of SEQ ID NO: 38 at one transposon end; and the other transposon flanking region comprises the nucleotide sequence of SEQ ID NO: 39 at the other transposon end; or (ii) an expression vector comprising said DNA transposon; wherein the heterologous polynucleotide comprises a sequence encoding a gene of interest, a complementary DNA (cDNA), a genome of interest or another genetic element, and wherein the heterologous polynucleotide further comprises a sequence encoding a selectable marker;
(b) introducing a recombinant transposase source into said eukaryotic cell, wherein the recombinant transposase source is selected from the group consisting of:
(i) an expression vector, wherein the expression vector (a) comprises a polynucleotide encoding the recombinant transposase of claim 7 or (b) encodes the recombinant transposase of claim 7;
(ii) an isolated mRNA encoding the recombinant transposase of claim 7; and
(iii) the recombinant transposase of claim 7; and
(c) culturing the eukaryotic cell in a medium under conditions to select for the selectable marker, wherein the DNA transposon is stably integrated into the genome of the eukaryotic cell.

16. A method for preparing a protein of interest, comprising
(a) into a eukaryotic cell a DNA molecule comprising (i) a DNA transposon comprising a heterologous polynucleotide flanked by transposon flanking regions, wherein one transposon flanking region comprises the nucleotide sequence of SEQ ID NO: 38 at one transposon end; and the other transposon flanking region comprises the nucleotide sequence of SEQ ID NO: 39 at the other transposon end; or (ii) an expression vector comprising said DNA transposon; wherein the heterologous polynucleotide comprises a sequence encoding at least one protein of interest and further a sequence encoding a selectable marker;
(b) introducing a recombinant transposase source into said eukaryotic cell, wherein the recombinant transposase source is selected from the group consisting of:
(i) an expression vector, wherein the expression vector (a) comprises a polynucleotide encoding the recombinant transposase of claim 7 or (b) encodes the recombinant transposase of claim 7;
(ii) an isolated mRNA encoding the recombinant transposase of claim 7; and
(iii) the recombinant transposase of claim 7;
(c) culturing culturing the eukaryotic cell in a medium under conditions to select for the selectable marker, wherein the heterologous polynucleotide comprising a sequence encoding the at least one protein of interest and a sequence encoding a selectable marker is integrated into the genome of the eukaryotic cell;
(d) optionally optionally isolating a single clone for clonal expansion to prepare a monoclonal cell line;
(e) culturing the eukaryotic cell under conditions to produce the protein of interest; and
(f) harvesting and optionally purifying the protein of interest.

17. A method for preparing a virus or virus like particle of interest, comprising
(a) into a eukaryotic cell a DNA molecule comprising (i) a DNA transposon comprising a heterologous polynucleotide flanked by transposon flanking regions, wherein one transposon flanking region comprises the nucleotide sequence of SEQ ID NO: 38 at one transposon end; and the other transposon flanking region comprises the nucleotide sequence of SEQ ID NO: 39 at the other transposon end; or (ii) an expression vector comprising said DNA transposon; wherein the heterologous polynucleotide comprises a sequence encoding at least one virus or virus like particle of interest and further a sequence encoding a selectable marker;
(b) introducing a recombinant transposase source into said eukaryotic cell, wherein the recombinant transposase source is selected from the group consisting of:
(i) an expression vector, wherein the expression vector (a) comprises a polynucleotide encoding the recombinant transposase of claim 7 or (b) encodes the recombinant transposase of claim 7;
(ii) an isolated mRNA encoding the recombinant transposase of claim 7; and
(iii) the recombinant transposase of claim 7;
(c) culturing the eukaryotic cell in a medium under conditions to select for the selectable marker, wherein the heterologous polynucleotide comprising a sequence encoding the at least one virus or virus like particle of interest and a sequence encoding a selectable marker is integrated into the genome of the eukaryotic cell;
(d) optionally isolating a single clone for clonal expansion to prepare a monoclonal cell line;
(e) culturing the eukaryotic cell under conditions to produce the virus or virus like particle of interest; and
(f) harvesting and optionally purifying the virus or virus like particle.

18. The recombinant transposase of claim 7, wherein the recombinant transposase comprises an amino acid sequence having at least 90% sequence identity with amino acids 10 to 585 of SEQ ID NO: 4 and at least one heterologous nuclear localization signal (NLS) fused to the transposase.

19. The recombinant transposase of claim 18, wherein the transposase is an Acyrthosiphon pisum transposase or an Aphis craccivora transposase.

20. The recombinant transposase of claim 8 wherein the at least one mutation is an amino acid substitution selected from the group consisting of K87Y, Q273V, V212I/I215L, I363V/K365S, K87Y/Q273V, K87Y/A264S/Q273V, A264S/Q273V, S270P/Q273V, K87Y/A264S, L583F, K576I, S372E, S277N and any combination thereof, and/or the at least one mutation is a deletion of N584 and/or E585, wherein the indicated amino acid position of the substitution and/or deletion corresponds to the amino acid position in the sequence of SEQ ID NO: 4, optionally wherein at least one heterologous NLS is fused to the transposase.

21. The eukaryotic cell of claim 14, wherein the eukaryotic cell is a mammalian cell.


SEQUENCE LISTING (85 sequences)

SEQ ID NO: 1 - DNA, 12 bp, synthetic construct
  source 1..12: mol_type = other DNA
cccttcgttagg

SEQ ID NO: 2 - DNA, 340 bp, Acyrthosiphon pisum
  source 1..340: mol_type = genomic DNA
ttaacccttcgttaggcgcgtggtctacaattgtagaccagcaataaatattgactattttagtatttataaatagcttgtaataccttgtactttatagtatctttactacagtgtaatcagtatgatcgtgaccgttgacgagtgttgtgtaataaaacatatcagtaatattatgagtggtctacaactgtagaccacgtgacttactaagttatttttataaattttgcaaaattcttatttttttttttattaggtacttctgtttttaatttcaacgaaacataaacagtaagtaaaaatatatgtatacgtaagctttttaatacattttgaa

SEQ ID NO: 3 - DNA, 415 bp, Acyrthosiphon pisum
  source 1..415: mol_type = genomic DNA
gcaccaaaacagttataatacctatatttgttcgacgataactggtaagaaaatataaaaatacattttaattttgtttcgggacttttgtaaacatgtaaatttaagatgtctatagttttcagactgattggaaggtaattgggtaagtagttctaaaaataaaaatgtcatgaaagaaattatgataatttcactataaacaagttaactgtattttcttcaaatattcatggattattttggatatttcaatgctcatactattctttatatgcatacaaataatttacattattaatataaacaaaaataattaaaattaattgaattggtcataaatgaaaatggtctacaggcgtagaccacgcgcctgaataagtaacaatatacctcgcgcctaacgaagggttaa

SEQ ID NO: 4 - protein, 585 aa, Acyrthosiphon pisum
  REGION 4..9: note = putative native nuclear localization signal
MSSQKKRRVDSTLDPSIVENWLNELSSCDEMSEADDESDNEEIDTTQKSDHESESEQDISENETNENVSDVQNNNFYLGKDKTTKWKKNKPNMQIRVRSHNIITHLPGPKQNARNAKFEIDCLKLFINENVIRLITISTNIYIESIKANFQRESDARLTDEREINAFIGILFLIGALRCARKNAHQIWDNSRGSGVEACYLAMSEKRFRFLVRCIRFDDINNRNERREIDKLAPIREVFEYFIANFQNNFIASEYLTVDEQLLAFRGRCSFKQYIPSKPAKYGLKIFALVDAKTAYTFNLEAYVGTQPEGPYKCKNSGEDIVLRIVQPVEGSNRNITADNWFTSFPLIKKLKHNKKLTYTGTIRKNKRELPSEFLPNKNRELHSSIFGFQEDYTLVSYCPKKNKAVLVVSSMHNDDTIDEENHAKKPEMITFYNKTKIGVDLVDQLCEKYNVARNTRRWPMVIFYDLLNISAINAICIYKANNLNSKISRRQFIENIAWELLKPQIKYRSTITKLPVELRRRARALLGIEEPSISVIPENLPNYVGRCYVCPRNKNKSTRRFCGQCRKYACKEHMKDICVNCLNE

SEQ ID NO: 5 - protein, 7 aa, synthetic construct
PKKKRKV

SEQ ID NO: 6 - protein, 594 aa, synthetic construct
  REGION 3..9: note = heterologous nuclear localization signal
MAPKKKRKVMSSQKKRRVDSTLDPSIVENWLNELSSCDEMSEADDESDNEEIDTTQKSDHESESEQDISENETNENVSDVQNNNFYLGKDKTTKWKKNKPNMQIRVRSHNIITHLPGPKQNARNAKFEIDCLKLFINENVIRLITISTNIYIESIKANFQRESDARLTDEREINAFIGILFLIGALRCARKNAHQIWDNSRGSGVEACYLAMSEKRFRFLVRCIRFDDINNRNERREIDKLAPIREVFEYFIANFQNNFIASEYLTVDEQLLAFRGRCSFKQYIPSKPAKYGLKIFALVDAKTAYTFNLEAYVGTQPEGPYKCKNSGEDIVLRIVQPVEGSNRNITADNWFTSFPLIKKLKHNKKLTYTGTIRKNKRELPSEFLPNKNRELHSSIFGFQEDYTLVSYCPKKNKAVLVVSSMHNDDTIDEENHAKKPEMITFYNKTKIGVDLVDQLCEKYNVARNTRRWPMVIFYDLLNISAINAICIYKANNLNSKISRRQFIENIAWELLKPQIKYRSTITKLPVELRRRARALLGIEEPSISVIPENLPNYVGRCYVCPRNKNKSTRRFCGQCRKYACKEHMKDICVNCLNE

SEQ ID NO: 7 - protein, 641 aa, synthetic construct
  REGION 2..23: note = Flag-tag
  REGION 26..32: note = heterologous nuclear localization signal
  REGION 33..40: note = linker
  REGION 626..641: note = heterologous nuclear localization signal
MDYKDHDGDYKDHDIDYKDDDDKMAPKKKRKVGIHGVPAAMSSQKKRRVDSTLDPSIVENWLNELSSCDEMSEADDESDNEEIDTTQKSDHESESEQDISENETNENVSDVQNNNFYLGKDKTTKWKKNKPNMQIRVRSHNIITHLPGPKQNARNAKFEIDCLKLFINENVIRLITISTNIYIESIKANFQRESDARLTDEREINAFIGILFLIGALRCARKNAHQIWDNSRGSGVEACYLAMSEKRFRFLVRCIRFDDINNRNERREIDKLAPIREVFEYFIANFQNNFIASEYLTVDEQLLAFRGRCSFKQYIPSKPAKYGLKIFALVDAKTAYTFNLEAYVGTQPEGPYKCKNSGEDIVLRIVQPVEGSNRNITADNWFTSFPLIKKLKHNKKLTYTGTIRKNKRELPSEFLPNKNRELHSSIFGFQEDYTLVSYCPKKNKAVLVVSSMHNDDTIDEENHAKKPEMITFYNKTKIGVDLVDQLCEKYNVARNTRRWPMVIFYDLLNISAINAICIYKANNLNSKISRRQFIENIAWELLKPQIKYRSTITKLPVELRRRARALLGIEEPSISVIPENLPNYVGRCYVCPRNKNKSTRRFCGQCRKYACKEHMKDICVNCLNEKRPAATKKAGQAKKKK

SEQ ID NO: 8 - DNA, 297 bp, synthetic construct
  source 1..297: mol_type = other DNA
ttaacccttcgttaggcgcgtggtctacaattgtagaccagcaataaatattgactattttagtatttataaatagcttgtaataccttgtactttatagtatctttactacagtgtaatcagtatgatcgtgaccgttgacgagtgttgtgtaataaaacatatcagtaatattatgagtggtctacaactgtagaccacgtgacttactaagttatttttataaattttgcaaaattcttatttttttttttattaggtacttctgtttttaatttcaacgaaacataaacagta

SEQ ID NO: 9 - DNA, 260 bp, synthetic construct
  source 1..260: mol_type = other DNA
ctaaaaataaaaatgtcatgaaagaaattatgataatttcactataaacaagttaactgtattttcttcaaatattcatggattattttggatatttcaatgctcatactattctttatatgcatacaaataatttacattattaatataaacaaaaataattaaaattaattgaattggtcataaatgaaaatggtctacaggcgtagaccacgcgcctgaataagtaacaatatacctcgcgcctaacgaagggttaa

SEQ ID NO: 10 - DNA, 214 bp, synthetic construct
  source 1..214: mol_type = other DNA
ttaacccttcgttaggcgcgtggtctacaattgtagaccagcaataaatattgactattttagtatttataaatagcttgtaataccttgtactttatagtatctttactacagtgtaatcagtatgatcgtgaccgttgacgagtgttgtgtaataaaacatatcagtaatattatgagtggtctacaactgtagaccacgtgacttactaag

SEQ ID NO: 11 - DNA, 154 bp, synthetic construct
  source 1..154: mol_type = other DNA
tactattctttatatgcatacaaataatttacattattaatataaacaaaaataattaaaattaattgaattggtcataaatgaaaatggtctacaggcgtagaccacgcgcctgaataagtaacaatatacctcgcgcctaacgaagggttaa

SEQ ID NO: 12 - DNA, 86 bp, synthetic construct
  source 1..86: mol_type = other DNA
ttaacccttcgttaggcgcgtggtctacaattgtagaccagcaataaatattgactattttagtatttataaatagcttgtaatac

SEQ ID NO: 13 - DNA, 57 bp, synthetic construct
  source 1..57: mol_type = other DNA
gcgtagaccacgcgcctgaataagtaacaatatacctcgcgcctaacgaagggttaa

SEQ ID NO: 14 - protein, 16 aa, synthetic construct
KRPAATKKAGQAKKKK

SEQ ID NO: 15 - DNA, 12 bp, synthetic construct
  source 1..12: mol_type = other DNA
cctaacgaaggg

SEQ ID NO: 16 - DNA, 1755 bp, Acyrthosiphon pisum
  source 1..1755: mol_type = other DNA
atgtcctcacagaagaagcggagagtggattcaaccctggacccttcaattgtggagaactggctcaacgagctgagtagctgcgatgaaatgtccgaggctgacgatgagtccgacaacgaagagatcgatactacacaaaagtccgatcacgaatccgaatcagagcaagatataagcgagaatgagactaatgaaaatgtatccgacgtacagaacaataatttctacctggggaaggataagactacaaagtggaagaagaacaaaccaaacatgcagatcagagtgcgatctcacaacataattacacacctgcctggtcccaaacagaacgcaaggaacgccaagttcgagattgattgtctgaaactctttatcaacgaaaatgtgatcagactgatcacaatctccactaacatctacatcgagagcattaaagctaatttccagcgcgagagcgatgctcgccttaccgatgagcgggaaatcaacgcctttatcgggatcctcttcctgatcggcgctctcagatgcgccagaaagaatgcccatcagatttgggacaactcccgcggatctggggtcgaggcctgttatctggctatgtctgagaaacgcttcagatttctggtgcgctgtattcggttcgacgatatcaataatcgtaatgagaggcgagagattgataaactggcccccatcagagaggtctttgagtacttcatagctaatttccagaacaacttcattgcatctgagtaccttacagtggacgagcagctgctggccttcagaggcaggtgtagctttaagcaatatatcccgtccaagcccgcaaagtacggcctgaagattttcgcattggttgacgccaagacagcctatacattcaatcttgaggcctacgtgggtacccagcccgagggaccttacaaatgtaagaatagcggcgaggatatcgtactaaggatcgtgcaacccgtcgagggcagcaaccgcaacatcactgctgacaattggttcacctcttttccactcatcaagaagttgaagcacaataagaagctgacttatacaggaactatccgtaagaataaaagagagctgcccagtgaatttctgcctaacaagaaccgggaactccacagcagcatctttggctttcaggaagattacactttggtaagttattgtcctaagaagaataaggcagtccttgtcgtctctagcatgcacaacgatgacactattgacgaggaaaaccatgcaaagaaacccgaaatgattacattctataacaagaccaaaattggggtcgatttggtggaccagctctgcgagaagtataatgtcgcaagaaacactagacgctggccaatggtgattttctatgatttgctgaacatttcagctattaatgccatttgcatttacaaggccaacaatctgaatagcaaaatctctagacgtcagttcatcgagaatatagcctgggagcttctgaagcctcaaattaagtataggtctaccatcacaaagctacccgtggagctgcgccgtagggctagagccctcttgggcattgaggagcccagcatttccgttattccagagaacctgccaaactacgtaggaagatgctacgtctgtccgcggaataagaataagagcactcggagattttgtggacaatgtcgcaagtatgcatgcaaggagcacatgaaggatatctgtgtaaactgccttaacgaa

SEQ ID NO: 17 - DNA, 1782 bp, synthetic construct
  source 1..1782: mol_type = other DNA
atggctcctaagaagaagcgcaaagtcatgtcctcacagaagaagcggagagtggattcaaccctggacccttcaattgtggagaactggctcaacgagctgagtagctgcgatgaaatgtccgaggctgacgatgagtccgacaacgaagagatcgatactacacaaaagtccgatcacgaatccgaatcagagcaagatataagcgagaatgagactaatgaaaatgtatccgacgtacagaacaataatttctacctggggaaggataagactacaaagtggaagaagaacaaaccaaacatgcagatcagagtgcgatctcacaacataattacacacctgcctggtcccaaacagaacgcaaggaacgccaagttcgagattgattgtctgaaactctttatcaacgaaaatgtgatcagactgatcacaatctccactaacatctacatcgagagcattaaagctaatttccagcgcgagagcgatgctcgccttaccgatgagcgggaaatcaacgcctttatcgggatcctcttcctgatcggcgctctcagatgcgccagaaagaatgcccatcagatttgggacaactcccgcggatctggggtcgaggcctgttatctggctatgtctgagaaacgcttcagatttctggtgcgctgtattcggttcgacgatatcaataatcgtaatgagaggcgagagattgataaactggcccccatcagagaggtctttgagtacttcatagctaatttccagaacaacttcattgcatctgagtaccttacagtggacgagcagctgctggccttcagaggcaggtgtagctttaagcaatatatcccgtccaagcccgcaaagtacggcctgaagattttcgcattggttgacgccaagacagcctatacattcaatcttgaggcctacgtgggtacccagcccgagggaccttacaaatgtaagaatagcggcgaggatatcgtactaaggatcgtgcaacccgtcgagggcagcaaccgcaacatcactgctgacaattggttcacctcttttccactcatcaagaagttgaagcacaataagaagctgacttatacaggaactatccgtaagaataaaagagagctgcccagtgaatttctgcctaacaagaaccgggaactccacagcagcatctttggctttcaggaagattacactttggtaagttattgtcctaagaagaataaggcagtccttgtcgtctctagcatgcacaacgatgacactattgacgaggaaaaccatgcaaagaaacccgaaatgattacattctataacaagaccaaaattggggtcgatttggtggaccagctctgcgagaagtataatgtcgcaagaaacactagacgctggccaatggtgattttctatgatttgctgaacatttcagctattaatgccatttgcatttacaaggccaacaatctgaatagcaaaatctctagacgtcagttcatcgagaatatagcctgggagcttctgaagcctcaaattaagtataggtctaccatcacaaagctacccgtggagctgcgccgtagggctagagccctcttgggcattgaggagcccagcatttccgttattccagagaacctgccaaactacgtaggaagatgctacgtctgtccgcggaataagaataagagcactcggagattttgtggacaatgtcgcaagtatgcatgcaaggagcacatgaaggatatctgtgtaaactgccttaacgaa

SEQ ID NO: 18 - DNA, 1923 bp, synthetic construct
  source 1..1923: mol_type = other DNA
atggactataaagatcacgatggcgattacaaggaccatgacatcgactacaaggatgacgacgacaaaatggccccaaagaaaaaacgaaaggtggggattcacggcgttcctgcagctatgtcctcacagaagaagcggagagtggattcaaccctggacccttcaattgtggagaactggctcaacgagctgagtagctgcgatgaaatgtccgaggctgacgatgagtccgacaacgaagagatcgatactacacaaaagtccgatcacgaatccgaatcagagcaagatataagcgagaatgagactaatgaaaatgtatccgacgtacagaacaataatttctacctggggaaggataagactacaaagtggaagaagaacaaaccaaacatgcagatcagagtgcgatctcacaacataattacacacctgcctggtcccaaacagaacgcaaggaacgccaagttcgagattgattgtctgaaactctttatcaacgaaaatgtgatcagactgatcacaatctccactaacatctacatcgagagcattaaagctaatttccagcgcgagagcgatgctcgccttaccgatgagcgggaaatcaacgcctttatcgggatcctcttcctgatcggcgctctcagatgcgccagaaagaatgcccatcagatttgggacaactcccgcggatctggggtcgaggcctgttatctggctatgtctgagaaacgcttcagatttctggtgcgctgtattcggttcgacgatatcaataatcgtaatgagaggcgagagattgataaactggcccccatcagagaggtctttgagtacttcatagctaatttccagaacaacttcattgcatctgagtaccttacagtggacgagcagctgctggccttcagaggcaggtgtagctttaagcaatatatcccgtccaagcccgcaaagtacggcctgaagattttcgcattggttgacgccaagacagcctatacattcaatcttgaggcctacgtgggtacccagcccgagggaccttacaaatgtaagaatagcggcgaggatatcgtactaaggatcgtgcaacccgtcgagggcagcaaccgcaacatcactgctgacaattggttcacctcttttccactcatcaagaagttgaagcacaataagaagctgacttatacaggaactatccgtaagaataaaagagagctgcccagtgaatttctgcctaacaagaaccgggaactccacagcagcatctttggctttcaggaagattacactttggtaagttattgtcctaagaagaataaggcagtccttgtcgtctctagcatgcacaacgatgacactattgacgaggaaaaccatgcaaagaaacccgaaatgattacattctataacaagaccaaaattggggtcgatttggtggaccagctctgcgagaagtataatgtcgcaagaaacactagacgctggccaatggtgattttctatgatttgctgaacatttcagctattaatgccatttgcatttacaaggccaacaatctgaatagcaaaatctctagacgtcagttcatcgagaatatagcctgggagcttctgaagcctcaaattaagtataggtctaccatcacaaagctacccgtggagctgcgccgtagggctagagccctcttgggcattgaggagcccagcatttccgttattccagagaacctgccaaactacgtaggaagatgctacgtctgtccgcggaataagaataagagcactcggagattttgtggacaatgtcgcaagtatgcatgcaaggagcacatgaaggatatctgtgtaaactgccttaacgaaaaacgccctgccgctacgaagaaagctgggcaggccaagaagaagaag

SEQ ID NO: 19 - DNA, 21 bp, synthetic construct
  source 1..21: mol_type = other DNA
cctaagaagaagcgcaaagtc

SEQ ID NO: 20 - DNA, 48 bp, synthetic construct
  source 1..48: mol_type = other DNA
aaacgccctgccgctacgaagaaagctgggcaggccaagaagaagaag

SEQ ID NO: 21 - DNA, 45 bp, synthetic construct
  source 1..45: mol_type = other DNA
ccaaagaaaaaacgaaaggtggggattcacggcgttcctgcagct

SEQ ID NO: 22 - protein, 15 aa, synthetic construct
PKKKRKVGIHGVPAA

SEQ ID NO: 23 - protein, 22 aa, synthetic construct
DYKDHDGDYKDHDIDYKDDDDK

SEQ ID NO: 24 - DNA, 66 bp, synthetic construct
  source 1..66: mol_type = other DNA
gactataaagatcacgatggcgattacaaggaccatgacatcgactacaaggatgacgacgacaaa

SEQ ID NO: 25 - protein, 6 aa, synthetic construct
QKKRRV

SEQ ID NO: 26 - protein, 585 aa, Aphis craccivora
MSSQKKRRVDSTLDPSIVENWLNELSSCDEMSEADDESDNEEIDTTQKSDHESESEQDMSENETNENVSDVQNNNFYLGKDKTTKWKKNKPNMQIRVRSHNIITHLPGPKQNARNAKFEIDCLKLFINDNVICLITISTNIYIESIKANFQRESDARLTDEREINAFIGILFLIGALRCARKNVHQIWDNSRGSGVEACYLAMSEKRFRFLVRCIRFDDINNRNERREIDKLAPIREVFEYFIANFQNNFIASEYLTVDEQLLAFRGRCSFKQYIPSKPAKYGLKMFALVDAKTAYTFNLEAYVGTQPEGPYKCKNSGEDIVLRLVQPVEGSNRNITADNWFTSFPLIKKLKHNKKLTYTGTIRKNKRELPSEFLPNKNRELHSSIFGFQEDYTLVSYCPKKNKAVLVVSSMHNDDTIDEENLAKKPEMITFYNKTKIGVDLVDQLCEKYNVARNTRRWPMVIFYDLLNISAINAICIYKANNLNSKISRRQFIENIAWELLKPQIEYRSTITKLPVELRRRARVLLSIEEPSISVIPENLPNYVGRCYVCPRNKNKSTRRFCGQCRKYACKEHMKDICVNCLNE

SEQ ID NO: 27 - DNA, 1755 bp, synthetic construct
  source 1..1755: mol_type = other DNA
atgtctagtcagaagaagcgccgggtggactctactctcgatccaagtatagttgaaaattggctaaacgagctctcttcatgcgatgaaatgtcagaagccgacgatgaatccgataacgaggaaatcgacactactcaaaagtccgatcatgaatccgaaagcgaacaagacatgtcagaaaacgaaactaacgagaatgtgagcgacgttcagaacaacaacttttaccttggaaaagacaaaaccacgaaatggaagaagaataaacccaatatgcaaatcagggtccgctctcataacataattactcatttgcccgggccaaaacaaaacgcaagaaacgcaaagtttgaaatcgactgcttgaagctgttcataaacgataacgtgatttgtctgatcactatcagcaccaatatctatatagaaagcataaaggctaacttccaacgcgagagcgacgctagactcacagatgaacgcgagattaatgccttcattgggatacttttccttattggcgccctgcggtgtgcacgcaagaacgttcatcaaatctgggacaacagcaggggctctggggtcgaagcctgctacctagccatgagtgaaaagaggtttcgcttccttgtgcggtgcattcgctttgacgatattaataaccgcaacgaacgccgagaaatagacaaactcgccccaattagagaggttttcgaatactttatcgccaattttcagaacaattttatagcatccgaatatctcactgttgacgagcaactgctcgccttcaggggcagatgctccttcaagcaatacattccctcaaaaccggctaaatacggcctcaagatgttcgcccttgttgacgccaaaactgcatatacatttaacttggaagcctatgtggggactcaacctgaaggtccttacaaatgtaagaacagcggagaagatatcgttctccggctcgtccaacctgtcgaaggatccaaccgaaatataaccgcagacaactggtttacatctttcccactgattaagaagctgaagcacaataagaaattgacatataccggcacgatacggaagaacaaacgggagctcccgtctgagtttctgccaaacaagaatcgggaacttcatagttcaattttcgggtttcaagaggattacactttggtgtcctactgccctaagaagaacaaagcagttctcgtagtgtcttccatgcataatgacgacactatcgatgaggagaacttggccaagaagcctgagatgattactttctacaacaaaacgaaaatcggggtcgaccttgtggatcaactgtgtgaaaagtataatgtagcccgtaatacgcgaaggtggcccatggtgatcttctacgacctgcttaacatcagtgcaatcaatgctatttgcatttacaaagcaaacaatctaaatagtaagatcagtcgaaggcaatttatcgagaacatcgcatgggaactactgaaaccccaaattgagtaccgctctacgatcactaaacttcccgttgagctgcgtcggcgcgctcgagtccttctttcaattgaggaaccatccatttccgtgatccctgaaaatcttccaaactatgtgggccgctgttatgtttgtcctcgaaataagaacaaaagcactcggcgtttctgcggtcaatgcaggaaatatgcctgtaaagaacatatgaaagatatttgcgtcaattgcctcaacgag

SEQ ID NO: 28 - DNA, 1923 bp, synthetic construct
  source 1..1923: mol_type = other DNA
atggactataaagatcacgatggcgattacaaggaccatgacatcgactacaaggatgacgacgacaaaatggccccaaagaaaaaacgaaaggtggggattcacggcgttcctgcagctatgtctagtcagaagaagcgccgggtggactctactctcgatccaagtatagttgaaaattggctaaacgagctctcttcatgcgatgaaatgtcagaagccgacgatgaatccgataacgaggaaatcgacactactcaaaagtccgatcatgaatccgaaagcgaacaagacatgtcagaaaacgaaactaacgagaatgtgagcgacgttcagaacaacaacttttaccttggaaaagacaaaaccacgaaatggaagaagaataaacccaatatgcaaatcagggtccgctctcataacataattactcatttgcccgggccaaaacaaaacgcaagaaacgcaaagtttgaaatcgactgcttgaagctgttcataaacgataacgtgatttgtctgatcactatcagcaccaatatctatatagaaagcataaaggctaacttccaacgcgagagcgacgctagactcacagatgaacgcgagattaatgccttcattgggatacttttccttattggcgccctgcggtgtgcacgcaagaacgttcatcaaatctgggacaacagcaggggctctggggtcgaagcctgctacctagccatgagtgaaaagaggtttcgcttccttgtgcggtgcattcgctttgacgatattaataaccgcaacgaacgccgagaaatagacaaactcgccccaattagagaggttttcgaatactttatcgccaattttcagaacaattttatagcatccgaatatctcactgttgacgagcaactgctcgccttcaggggcagatgctccttcaagcaatacattccctcaaaaccggctaaatacggcctcaagatgttcgcccttgttgacgccaaaactgcatatacatttaacttggaagcctatgtggggactcaacctgaaggtccttacaaatgtaagaacagcggagaagatatcgttctccggctcgtccaacctgtcgaaggatccaaccgaaatataaccgcagacaactggtttacatctttcccactgattaagaagctgaagcacaataagaaattgacatataccggcacgatacggaagaacaaacgggagctcccgtctgagtttctgccaaacaagaatcgggaacttcatagttcaattttcgggtttcaagaggattacactttggtgtcctactgccctaagaagaacaaagcagttctcgtagtgtcttccatgcataatgacgacactatcgatgaggagaacttggccaagaagcctgagatgattactttctacaacaaaacgaaaatcggggtcgaccttgtggatcaactgtgtgaaaagtataatgtagcccgtaatacgcgaaggtggcccatggtgatcttctacgacctgcttaacatcagtgcaatcaatgctatttgcatttacaaagcaaacaatctaaatagtaagatcagtcgaaggcaatttatcgagaacatcgcatgggaactactgaaaccccaaattgagtaccgctctacgatcactaaacttcccgttgagctgcgtcggcgcgctcgagtccttctttcaattgaggaaccatccatttccgtgatccctgaaaatcttccaaactatgtgggccgctgttatgtttgtcctcgaaataagaacaaaagcactcggcgtttctgcggtcaatgcaggaaatatgcctgtaaagaacatatgaaagatatttgcgtcaattgcctcaacgagaaacgccctgccgctacgaagaaagctgggcaggccaagaagaagaag

SEQ ID NO: 29 - DNA, 362 bp, Aphis craccivora
  source 1..362: mol_type = genomic DNA
ttaacccttcgttaggcgcgtggtctacaattgtagaccagctataaatattgacaattttagtatttataaaaagcttgtagtatcttgtactttatagtagctgttgttcatctttactacagtgcaatcagtatgaccgtgaccgctgacgagcgttgtgttacgaaacatatcaacaatattaggagtggtctacaatcgtagaccacgcgacttgctaagttatatttttttataaatttgcaaaattcttattttttttttaattaagtacatttgtttttaatatttttaatttcaacgaaacataaacagtaagtaaaaatatatgtatacgtaaacattttaatgcatttgga

SEQ ID NO: 30 - DNA, 407 bp, Aphis craccivora
  source 1..407: mol_type = genomic DNA
acagttataatatctatatttgttcgacgataactggtaagaaaaaataaaaataaattttaactttgtttcgggtcttttgtaaacatgtcaatttaagatatctatagttttcagactgattggaaggtaattgggtaagtagttctaaaaataaaaatgtcatgaaagaaattatgataatttcactataaataagttaactgtattttcttcaaatgtacatggattattttggatatttcaatgttcatacgattctttatatgcatacaagtaatttacattattaatataaacaaaaataattaaaattaattgaattggtcataaattaaaatggtctacaagtgtagaccaagcgcctgaataagtaacaatatacctcgcgcctaacgaagggttaa

SEQ ID NO: 31 - DNA, 1854 bp, synthetic construct
  source 1..1854: mol_type = other DNA
atggccccaaagaaaaaacgaaaggtggggattcacggcgttcctgcagctatgtcctcacagaagaagcggagagtggattcaaccctggacccttcaattgtggagaactggctcaacgagctgagtagctgcgatgaaatgtccgaggctgacgatgagtccgacaacgaagagatcgatactacacaaaagtccgatcacgaatccgaatcagagcaagatataagcgagaatgagactaatgaaaatgtatccgacgtacagaacaataatttctacctggggaaggataagactacaaagtggaagaagaacaaaccaaacatgcagatcagagtgcgatctcacaacataattacacacctgcctggtcccaaacagaacgcaaggaacgccaagttcgagattgattgtctgaaactctttatcaacgaaaatgtgatcagactgatcacaatctccactaacatctacatcgagagcattaaagctaatttccagcgcgagagcgatgctcgccttaccgatgagcgggaaatcaacgcctttatcgggatcctcttcctgatcggcgctctcagatgcgccagaaagaatgcccatcagatttgggacaactcccgcggatctggggtcgaggcctgttatctggctatgtctgagaaacgcttcagatttctggtgcgctgtattcggttcgacgatatcaataatcgtaatgagaggcgagagattgataaactggcccccatcagagaggtctttgagtacttcatagctaatttccagaacaacttcattgcatctgagtaccttacagtggacgagcagctgctggccttcagaggcaggtgtagctttaagcaatatatcccgtccaagcccgcaaagtacggcctgaagattttcgcattggttgacgccaagacagcctatacattcaatcttgaggcctacgtgggtacccagcccgagggaccttacaaatgtaagaatagcggcgaggatatcgtactaaggatcgtgcaacccgtcgagggcagcaaccgcaacatcactgctgacaattggttcacctcttttccactcatcaagaagttgaagcacaataagaagctgacttatacaggaactatccgtaagaataaaagagagctgcccagtgaatttctgcctaacaagaaccgggaactccacagcagcatctttggctttcaggaagattacactttggtaagttattgtcctaagaagaataaggcagtccttgtcgtctctagcatgcacaacgatgacactattgacgaggaaaaccatgcaaagaaacccgaaatgattacattctataacaagaccaaaattggggtcgatttggtggaccagctctgcgagaagtataatgtcgcaagaaacactagacgctggccaatggtgattttctatgatttgctgaacatttcagctattaatgccatttgcatttacaaggccaacaatctgaatagcaaaatctctagacgtcagttcatcgagaatatagcctgggagcttctgaagcctcaaattaagtataggtctaccatcacaaagctacccgtggagctgcgccgtagggctagagccctcttgggcattgaggagcccagcatttccgttattccagagaacctgccaaactacgtaggaagatgctacgtctgtccgcggaataagaataagagcactcggagattttgtggacaatgtcgcaagtatgcatgcaaggagcacatgaaggatatctgtgtaaactgccttaacgaaaaacgccctgccgctacgaagaaagctgggcaggccaagaagaagaag

SEQ ID NO: 32 - protein, 618 aa, synthetic construct
MAPKKKRKVGIHGVPAAMSSQKKRRVDSTLDPSIVENWLNELSSCDEMSEADDESDNEEIDTTQKSDHESESEQDISENETNENVSDVQNNNFYLGKDKTTKWKKNKPNMQIRVRSHNIITHLPGPKQNARNAKFEIDCLKLFINENVIRLITISTNIYIESIKANFQRESDARLTDEREINAFIGILFLIGALRCARKNAHQIWDNSRGSGVEACYLAMSEKRFRFLVRCIRFDDINNRNERREIDKLAPIREVFEYFIANFQNNFIASEYLTVDEQLLAFRGRCSFKQYIPSKPAKYGLKIFALVDAKTAYTFNLEAYVGTQPEGPYKCKNSGEDIVLRIVQPVEGSNRNITADNWFTSFPLIKKLKHNKKLTYTGTIRKNKRELPSEFLPNKNRELHSSIFGFQEDYTLVSYCPKKNKAVLVVSSMHNDDTIDEENHAKKPEMITFYNKTKIGVDLVDQLCEKYNVARNTRRWPMVIFYDLLNISAINAICIYKANNLNSKISRRQFIENIAWELLKPQIKYRSTITKLPVELRRRARALLGIEEPSISVIPENLPNYVGRCYVCPRNKNKSTRRFCGQCRKYACKEHMKDICVNCLNEKRPAATKKAGQAKKKK

SEQ ID NO: 33 - DNA, 1854 bp, synthetic construct
  source 1..1854: mol_type = other DNA
atggccccaaagaaaaaacgaaaggtggggattcacggcgttcctgcagctatgtcctcacagaagaagcggagagtggattcaaccctggacccttcaattgtggagaactggctcaacgagctgagtagctgcgatgaaatgtccgaggctgacgatgagtccgacaacgaagagatcgatactacacaaaagtccgatcacgaatccgaatcagagcaagatataagcgagaatgagactaatgaaaatgtatccgacgtacagaacaataatttctacctggggaaggataagactacaaagtggtacaagaacaaaccaaacatgcagatcagagtgcgatctcacaacataattacacacctgcctggtcccaaacagaacgcaaggaacgccaagttcgagattgattgtctgaaactctttatcaacgaaaatgtgatcagactgatcacaatctccactaacatctacatcgagagcattaaagctaatttccagcgcgagagcgatgctcgccttaccgatgagcgggaaatcaacgcctttatcgggatcctcttcctgatcggcgctctcagatgcgccagaaagaatgcccatcagatttgggacaactcccgcggatctggggtcgaggcctgttatctggctatgtctgagaaacgcttcagatttctggtgcgctgtattcggttcgacgatatcaataatcgtaatgagaggcgagagattgataaactggcccccatcagagaggtctttgagtacttcatagctaatttccagaacaacttcattgcatctgagtaccttacagtggacgagcagctgctggccttcagaggcaggtgtagctttaagcaatatatcccgtccaagcccgcaaagtacggcctgaagattttcgcattggttgacgccaagacagcctatacattcaatcttgaggcctacgtgggtacccagcccgagggaccttacaaatgtaagaatagcggcgaggatatcgtactaaggatcgtgcaacccgtcgagggcagcaaccgcaacatcactgctgacaattggttcacctcttttccactcatcaagaagttgaagcacaataagaagctgacttatacaggaactatccgtaagaataaaagagagctgcccagtgaatttctgcctaacaagaaccgggaactccacagcagcatctttggctttcaggaagattacactttggtaagttattgtcctaagaagaataaggcagtccttgtcgtctctagcatgcacaacgatgacactattgacgaggaaaaccatgcaaagaaacccgaaatgattacattctataacaagaccaaaattggggtcgatttggtggaccagctctgcgagaagtataatgtcgcaagaaacactagacgctggccaatggtgattttctatgatttgctgaacatttcagctattaatgccatttgcatttacaaggccaacaatctgaatagcaaaatctctagacgtcagttcatcgagaatatagcctgggagcttctgaagcctcaaattaagtataggtctaccatcacaaagctacccgtggagctgcgccgtagggctagagccctcttgggcattgaggagcccagcatttccgttattccagagaacctgccaaactacgtaggaagatgctacgtctgtccgcggaataagaataagagcactcggagattttgtggacaatgtcgcaagtatgcatgcaaggagcacatgaaggatatctgtgtaaactgccttaacgaaaaacgccctgccgctacgaagaaagctgggcaggccaagaagaagaag

SEQ ID NO: 34 - protein, 618 aa, synthetic construct
MAPKKKRKVGIHGVPAAMSSQKKRRVDSTLDPSIVENWLNELSSCDEMSEADDESDNEEIDTTQKSDHESESEQDISENETNENVSDVQNNNFYLGKDKTTKWYKNKPNMQIRVRSHNIITHLPGPKQNARNAKFEIDCLKLFINENVIRLITISTNIYIESIKANFQRESDARLTDEREINAFIGILFLIGALRCARKNAHQIWDNSRGSGVEACYLAMSEKRFRFLVRCIRFDDINNRNERREIDKLAPIREVFEYFIANFQNNFIASEYLTVDEQLLAFRGRCSFKQYIPSKPAKYGLKIFALVDAKTAYTFNLEAYVGTQPEGPYKCKNSGEDIVLRIVQPVEGSNRNITADNWFTSFPLIKKLKHNKKLTYTGTIRKNKRELPSEFLPNKNRELHSSIFGFQEDYTLVSYCPKKNKAVLVVSSMHNDDTIDEENHAKKPEMITFYNKTKIGVDLVDQLCEKYNVARNTRRWPMVIFYDLLNISAINAICIYKANNLNSKISRRQFIENIAWELLKPQIKYRSTITKLPVELRRRARALLGIEEPSISVIPENLPNYVGRCYVCPRNKNKSTRRFCGQCRKYACKEHMKDICVNCLNEKRPAATKKAGQAKKKK

SEQ ID NO: 35 - protein, 618 aa, synthetic construct
MAPKKKRKVGIHGVPAAMSSQKKRRVDSTLDPSIVENWLNELSSCDEMSEADDESDNEEIDTTQKSDHESESEQDISENETNENVSDVQNNNFYLGKDKTTKWKKNKPNMQIRVRSHNIITHLPGPKQNARNAKFEIDCLKLFINENVIRLITISTNIYIESIKANFQRESDARLTDEREINAFIGILFLIGALRCARKNAHQIWDNSRGSGVEACYLAMSEKRFRFLVRCIRFDDINNRNERREIDKLAPIREVFEYFIANFQNNFIASEYLTVDEQLLAFRGRCSFKQYIPSKPAKYGLKIFALVDAKTAYTFNLEAYVGTQPEGPYKCKNSGEDIVLRIVQPVEGSNRNITADNWFTSFPLIKKLKHNKKLTYTGTVRSNKRELPSEFLPNKNRELHSSIFGFQEDYTLVSYCPKKNKAVLVVSSMHNDDTIDEENHAKKPEMITFYNKTKIGVDLVDQLCEKYNVARNTRRWPMVIFYDLLNISAINAICIYKANNLNSKISRRQFIENIAWELLKPQIKYRSTITKLPVELRRRARALLGIEEPSISVIPENLPNYVGRCYVCPRNKNKSTRRFCGQCRKYACKEHMKDICVNCLNEKRPAATKKAGQAKKKK

SEQ ID NO: 36 - protein, 618 aa, synthetic construct
MAPKKKRKVGIHGVPAAMSSQKKRRVDSTLDPSIVENWLNELSSCDEMSEADDESDNEEIDTTQKSDHESESEQDISENETNENVSDVQNNNFYLGKDKTTKWKKNKPNMQIRVRSHNIITHLPGPKQNARNAKFEIDCLKLFINENVIRLITISTNIYIESIKANFQRESDARLTDEREINAFIGILFLIGALRCARKNAHQIWDNSRGSGVEACYLAMSEKRFRFLVRCIRFDDINNRNERREIDKLAPIREVFEYFIANFQNNFIASEYLTVDEQLLSFRGRCSFKVYIPSKPAKYGLKIFALVDAKTAYTFNLEAYVGTQPEGPYKCKNSGEDIVLRIVQPVEGSNRNITADNWFTSFPLIKKLKHNKKLTYTGTIRKNKRELPSEFLPNKNRELHSSIFGFQEDYTLVSYCPKKNKAVLVVSSMHNDDTIDEENHAKKPEMITFYNKTKIGVDLVDQLCEKYNVARNTRRWPMVIFYDLLNISAINAICIYKANNLNSKISRRQFIENIAWELLKPQIKYRSTITKLPVELRRRARALLGIEEPSISVIPENLPNYVGRCYVCPRNKNKSTRRFCGQCRKYACKEHMKDICVNCLNEKRPAATKKAGQAKKKK

SEQ ID NO: 37 - protein, 618 aa, synthetic construct
MAPKKKRKVGIHGVPAAMSSQKKRRVDSTLDPSIVENWLNELSSCDEMSEADDESDNEEIDTTQKSDHESESEQDISENETNENVSDVQNNNFYLGKDKTTKWYKNKPNMQIRVRSHNIITHLPGPKQNARNAKFEIDCLKLFINENVIRLITISTNIYIESIKANFQRESDARLTDEREINAFIGILFLIGALRCARKNAHQIWDNSRGSGVEACYLAMSEKRFRFLVRCIRFDDINNRNERREIDKLAPIREVFEYFIANFQNNFIASEYLTVDEQLLSFRGRCSFKQYIPSKPAKYGLKIFALVDAKTAYTFNLEAYVGTQPEGPYKCKNSGEDIVLRIVQPVEGSNRNITADNWFTSFPLIKKLKHNKKLTYTGTIRKNKRELPSEFLPNKNRELHSSIFGFQEDYTLVSYCPKKNKAVLVVSSMHNDDTIDEENHAKKPEMITFYNKTKIGVDLVDQLCEKYNVARNTRRWPMVIFYDLLNISAINAICIYKANNLNSKISRRQFIENIAWELLKPQIKYRSTITKLPVELRRRARALLGIEEPSISVIPENLPNYVGRCYVCPRNKNKSTRRFCGQCRKYACKEHMKDICVNCLNEKRPAATKKAGQAKKKK

SEQ ID NO: 38 - DNA, 16 bp, synthetic construct
  source 1..16: mol_type = other DNA
ttaacccttcgttagg

SEQ ID NO: 39 - DNA, 16 bp, synthetic construct
  source 1..16: mol_type = other DNA
cctaacgaagggttaa

SEQ ID NO: 40 - DNA, 20 bp, synthetic construct
  source 1..20: mol_type = other DNA
ttaacccttcgttaggcgcg

SEQ ID NO: 41 - DNA, 20 bp, synthetic construct
  source 1..20: mol_type = other DNA
cgcgcctaacgaagggttaa

SEQ ID NO: 42 - skipped

SEQ ID NO: 43 - DNA, 20 bp, synthetic construct
  source 1..20: mol_type = other DNA
tggtctacannngtagacca

SEQ ID NO: 44 - DNA, 20 bp, synthetic construct
  source 1..20: mol_type = other DNA
tggtctacarbygtagacca

SEQ ID NO: 45 - DNA, 40 bp, synthetic construct
  source 1..40: mol_type = other DNA
ttaacccttcgttaggcgcgtggtctacannngtagacca

SEQ ID NO: 46 - DNA, 40 bp, synthetic construct
  source 1..40: mol_type = other DNA
ttaacccttcgttaggcgcgtggtctacaattgtagacca

SEQ ID NO: 47 - DNA, 67 bp, synthetic construct
  source 1..67: mol_type = other DNA
tggtctacannngtagaccamgcgcctgaataagtaacaatatacctcgcgcctaacgaagggttaa

SEQ ID NO: 48 - DNA, 67 bp, synthetic construct
  source 1..67: mol_type = other DNA
tggtctacannngtagaccacgcgcctgaataagtaacaatatacctcgcgcctaacgaagggttaa

SEQ ID NO: 49 - DNA, 67 bp, synthetic construct
  source 1..67: mol_type = other DNA
tggtctacaggcgtagaccacgcgcctgaataagtaacaatatacctcgcgcctaacgaagggttaa

SEQ ID NO: 50-51 - skipped (2 entries)

SEQ ID NO: 52 - protein, 8 aa, synthetic construct
GIHGVPAA

SEQ ID NO: 53 - DNA, 95 bp, synthetic construct
  source 1..95: mol_type = other DNA
ttaacccttcgttaggcgcgtggtctacaattgtagaccaaggcgcgaggtatattgttacttattcaggcgcgtggtctacgcctgtagaccag

SEQ ID NO: 54 - DNA, 54 bp, synthetic construct
  source 1..54: mol_type = other DNA
tggtctacaggcgtagaccacgcgcctgaataagtaacaatatacctcgcgcct

SEQ ID NO: 55 - DNA, 394 bp, synthetic construct
  source 1..394: mol_type = other DNA
ttaacccttcgttaggcgcgtggtctacaattgtagaccaaggcgcgaggtatattgttacttattcaggcgcgtggtctacgcctgtagaccagcaataaatattgactattttagtatttataaatagcttgtaataccttgtactttatagtatctttactacagtgtaatcagtatgatcgtgaccgttgacgagtgttgtgtaataaaacatatcagtaatattatgagtggtctacaactgtagaccacgtgacttactaagttatttttataaattttgcaaaattcttatttttttttttattaggtacttctgtttttaatttcaacgaaacataaacagtaagtaaaaatatatgtatacgtaagctttttaatacattttgaa

SEQ ID NO: 56 - DNA, 469 bp, synthetic construct
  source 1..469: mol_type = other DNA
gcaccaaaacagttataatacctatatttgttcgacgataactggtaagaaaatataaaaatacattttaattttgtttcgggacttttgtaaacatgtaaatttaagatgtctatagttttcagactgattggaaggtaattgggtaagtagttctaaaaataaaaatgtcatgaaagaaattatgataatttcactataaacaagttaactgtattttcttcaaatattcatggattattttggatatttcaatgctcatactattctttatatgcatacaaataatttacattattaatataaacaaaaataattaaaattaattgaattggtcataaatgaaaatggtctacaggcgtagaccacgcgcctgaataagtaacaatatacctcgcgccttggtctacaggcgtagaccacgcgcctgaataagtaacaatatacctcgcgcctaacgaagggttaa

SEQ ID NO: 57 - DNA, 401 bp, synthetic construct
  source 1..401: mol_type = other DNA
ttaacccttcgttaggcgcgtggtctacaattgtagaccaaggcgcgaggtatattgttacttattcaggcgcgtggtctacgcctgtagaccaaggcgcggcaataaatattgactattttagtatttataaatagcttgtaataccttgtactttatagtatctttactacagtgtaatcagtatgatcgtgaccgttgacgagtgttgtgtaataaaacatatcagtaatattatgagtggtctacaactgtagaccacgtgacttactaagttatttttataaattttgcaaaattcttatttttttttttattaggtacttctgtttttaatttcaacgaaacataaacagtaagtaaaaatatatgtatacgtaagctttttaatacattttgaa

SEQ ID NO: 58 - DNA, 200 bp, synthetic construct
  source 1..200: mol_type = other DNA
ttaacccttcgttaggcgcgtggtctacaattgtagaccagcaataaatattgactattttagtatttataaatagcttgtaataccttgtactttatagtatctttactacagtgtaatcagtatgatcgtgaccgttgacgagtgttgtgtaataaaacatatcagtaatattatgagtggtctacaactgtagacca

SEQ ID NO: 59 - DNA, 77 bp, synthetic construct
  source 1..77: mol_type = other DNA
taaatgaaaatggtctacaggcgtagaccacgcgcctgaataagtaacaatatacctcgcgcctaacgaagggttaa

SEQ ID NO: 60 - DNA, 67 bp, synthetic construct
  source 1..67: mol_type = other DNA
tggtctacannngtagaccaagcgcctgaataagtaacaatatacctcgcgcctaacgaagggttaa

SEQ ID NO: 61 - DNA, 67 bp, synthetic construct
  source 1..67: mol_type = other DNA
tggtctacaagtgtagaccaagcgcctgaataagtaacaatatacctcgcgcctaacgaagggttaa

SEQ ID NO: 62 - DNA, 320 bp, synthetic construct
  source 1..320: mol_type = other DNA
ttaacccttcgttaggcgcgtggtctacaattgtagaccagctataaatattgacaattttagtatttataaaaagcttgtagtatcttgtactttatagtagctgttgttcatctttactacagtgcaatcagtatgaccgtgaccgctgacgagcgttgtgttacgaaacatatcaacaatattaggagtggtctacaatcgtagaccacgcgacttgctaagttatatttttttataaatttgcaaaattcttattttttttttaattaagtacatttgtttttaatatttttaatttcaacgaaacataaacagta

SEQ ID NO: 63 - DNA, 225 bp, synthetic construct
  source 1..225: mol_type = other DNA
ttaacccttcgttaggcgcgtggtctacaattgtagaccagctataaatattgacaattttagtatttataaaaagcttgtagtatcttgtactttatagtagctgttgttcatctttactacagtgcaatcagtatgaccgtgaccgctgacgagcgttgtgttacgaaacatatcaacaatattaggagtggtctacaatcgtagaccacgcgacttgctaag

SEQ ID NO: 64 - DNA, 211 bp, synthetic construct
  source 1..211: mol_type = other DNA
ttaacccttcgttaggcgcgtggtctacaattgtagaccagctataaatattgacaattttagtatttataaaaagcttgtagtatcttgtactttatagtagctgttgttcatctttactacagtgcaatcagtatgaccgtgaccgctgacgagcgttgtgttacgaaacatatcaacaatattaggagtggtctacaatcgtagacca

SEQ ID NO: 65 - DNA, 260 bp, synthetic construct
  source 1..260: mol_type = other DNA
ctaaaaataaaaatgtcatgaaagaaattatgataatttcactataaataagttaactgtattttcttcaaatgtacatggattattttggatatttcaatgttcatacgattctttatatgcatacaagtaatttacattattaatataaacaaaaataattaaaattaattgaattggtcataaattaaaatggtctacaagtgtagaccaagcgcctgaataagtaacaatatacctcgcgcctaacgaagggttaa

SEQ ID NO: 66 - DNA, 154 bp, synthetic construct
  source 1..154: mol_type = other DNA
tacgattctttatatgcatacaagtaatttacattattaatataaacaaaaataattaaaattaattgaattggtcataaattaaaatggtctacaagtgtagaccaagcgcctgaataagtaacaatatacctcgcgcctaacgaagggttaa

SEQ ID NO: 67 - DNA, 77 bp, synthetic construct
  source 1..77: mol_type = other DNA
taaattaaaatggtctacaagtgtagaccaagcgcctgaataagtaacaatatacctcgcgcctaacgaagggttaa

SEQ ID NO: 68 - DNA, 27 bp, synthetic construct
  source 1..27: mol_type = other DNA
tggtctacannngtagaccamgcgcct

SEQ ID NO: 69 - DNA, 27 bp, synthetic construct
  source 1..27: mol_type = other DNA
tggtctacannngtagaccacgcgcct

SEQ ID NO: 70 - DNA, 27 bp, synthetic construct
  source 1..27: mol_type = other DNA
tggtctacannngtagaccaagcgcct

SEQ ID NO: 71 - protein, 605 aa, Aplysia californica
MANRPRRMLTTLQAVQEVYRDLDSGDEDLDDDASPNWNGHDSDDVDSSSDHDQEQEEEQADEREETRFEDVPGPSTSASRGRGRGRGRGSGRGRGRGAGGTRRTTTNTREWTRDVGNFRPVIADFTAAPGIKVDVSNFTPSQFFSLFVDDDLLRSLVIETKRYAEQYLAATPLKQHSRANQWHNTDVTEMKCFIGVFLLMGMIKKPEINSYWCTDPLFTTPVFNAALSRDRFLLLLKFLHFSDNTNMPNVQDRNRDRLYKIRPVLNHLFERFQHVYVPDQNVAIDESLMLWKGRLLFKQYIPLKRARFGIKSFLLCDGSGYTYRFRIYAGKDESTEAIDEMLPDECATFGRPAKEVVYLMLPLLNKGYKLFVDNWYTSVDLFSFLHSQQTSACGTMRKNRTAPLLRALHVEKGATESCANGHLLAQKYHDKREVYMLTTCHEPQGTTEKPPSVLSYNVNMGSVDRHDQLLQPYEATRKILKWYKKLFVRMVQVAMLNAHILYEKAGNKSTFLDFQKDVVSDWIFGDDGAPSVTDDHAVRLYDRHFIEVLPPTEKKVHPQKRCRVCTKKSLRKDTRYYCLDCPSKPGLCLSGCFKKYHTERFYWRN

SEQ ID NO: 72 - DNA, 311 bp, Aplysia californica
  source 1..311: mol_type = genomic DNA
ttaaccccttcactgctacgacgacgtagtacctacgtctccctgaacgcgcctctgtgtgctacagcgacgtgactactacgtcctgcattatccctgttttgtttcgttccctgctaaagtctaatttgcttttataatatttagagtaggttcccttgaaagctcatttattctacgagttagaaatccactgttcgtgggaaaatttctgctactggcctagttctagcgggaaatagaagcgtcctttccgagttttcgtctgctctagtacgcaccgtatcgaacgctcagctcagctaacgaag

SEQ ID NO: 73 - DNA, 423 bp, Aplysia californica
  source 1..423: mol_type = genomic DNA
gagtgaagtccagtttcgctagtgattgtaaatagttgtgtacatatctctgtgctttattttttctgtggaattgtgtgtgcatataagtgtagtgtgttattatttctgtatggattaccctacaagttgaataccagttccagtcttccctactagagcaaggtaagaaaacattttttcccctatcttatgcaatcttctactcctctcaaaatctttttccttccaataatccccctgtacatagtctacttggaaagaccattcatttctctacaagaaaatatatactttgttatacttttcacttcattcaagagcaccaaagaccaataagggaagtcacctatggtttcatgcggtacctggaaatttttccagcatatgggaagtcaaagtggacagcagtgaaggggttaa

SEQ ID NO: 74 - protein, 628 aa, Onthophagus taurus
MSSKRPLSELELQMEIERIMNGEESNSELYSDSGSDWEEDNLEVDESISDDDDDTNSDEDVRTARHDSPVFSTQETRNVPIDVADVRSSTVVPQNVEQETDQNLPLSKIPRKEKSIILPNTTNLKGKNGHKWSAKLPLRSKRTASRNIVHFLVGSKGKAKECLTVEDHFLHFISNDILNIILQHTNEEITRQSAKYAKAEAWISHVSKEEIMAFFAVLLLSAVKKDNHLHVKRMFDSAISGSFYKSCMCYERFNFLVNCLRFDDKESREARKQDDPFTHIRKIWEIFIDTCRTSYTPSSYLTIDEQLLGFRGRCPFKMYIPTKPAKYGIKIVMLCDSSSKYLVDACPYLGKGTQTGGLPVANYFVKELTKSVHGSNRNITMDNWFTSVDLADELLQKPYKLTMLGTIKKNKREIPPQLLETKGRNPKTSMFCFDRNKTLVSYEPKKNKIVLLLSTMHDGAEISEENGKPMMILNYNETKGGVDTFDQLSSNMSCSRKTRRWPLCVFYGMVNSASINSYIVYCYNTFKRGEKPLSREGFMIELAYRLAKPWMEHRISVPSLRRNLRQNICEILKVDVVDENTVPQDKKRTICFYCPSKLRRMTTTYCVQCRKAFCGTHRGNYCTECASH

SEQ ID NO: 75 - DNA, 324 bp, Onthophagus taurus
  source 1..324: mol_type = genomic DNA
ttaaccctagaatactattccttcgtaaaatttaccgcggtagggtattcccgctagttttccggttctttctagtgattgcccgtttcaacgttctcagtagcttcatagctagtcgtgagctatacgtcagtgtaggtgaccgtgcgtaattgttttccagtattggttatttcgaaaggttgtcgtcaattttacgacggaatagtgttgacgtatgttcgcgaaatttgtacaattttcgagagattcaatttttctaacaagtaagagaattttatcctaatttattatgttcctaatactaatcgatcttttagtaaa

SEQ ID NO: 76 - DNA, 179 bp, Onthophagus taurus
  source 1..179: mol_type = genomic DNA
gataatttaggtttactatatattattgtgttactgaatacgtaagcaagatatcttcatagattgtacatattttgtaataaaaatgcgctttctatataatgttttttaatgatcgtcaagtttacgacagtatagtattcacgtatatcaaaaccagtatagtattctagggttaa

SEQ ID NO: 77 - protein, 628 aa, Onthophagus taurus
MSSKRPLSELELQMEIERIMNGEESNSELYSDSGSDWEEDNLEVDESISDDDDDTNSDEDVRTARHDSPVFSTQETRNVPIDVADVRSSTVVPQNVEQETDQNLPLSKIPRKEKSIILPNTTNLKGKNGHKWSAKLPLRSKRTASRNIVHFLVGSKGKAKECLTVEDHFLHFISNDILNIILQHTNEEITRQSAKYAKAEAWISHVSKEEIMAFFAVLLLSAVKKDNHLHVKRMFDSAISGSFYKSCMCYERFNFLVNCLRFDDKESREARKQDDPFTHIRKIWEIFIDTCRTSYTPSSYLTIDEQLLGFRGRCPFKMYIPTKPAKYGIKIVMLCDSSSKYLVDACPHLGKGTQTGGLSVANYFVKELTKSVHGSNRNITMDNWFTSVDLADELLQKPYKLTMLGTIKKNKREIPPQLLETKGRNPKTSMFCFDRNKTLVSYEPKKNKIVLLLSTMHDGAEISEENGKPMMILNYNETKGGVDTFDQLSSNMSCSRKTRRWPLCVFYGMVNSASINSYIVYCYNTFKRGEKPLSREGFMIELAYRLAKPWMEHRISVPSLRRNLRQNICEILKVDVVDENTVPQDKKRTICFYCPSKLRRMTTTYCVQCRKAFCGTHRGNYCTECASH

SEQ ID NO: 78 - DNA, 324 bp, Onthophagus taurus; identical to SEQ ID NO: 75
  source 1..324: mol_type = genomic DNA

SEQ ID NO: 79 - DNA, 179 bp, Onthophagus taurus; identical to SEQ ID NO: 76
  source 1..179: mol_type = genomic DNA

SEQ ID NO: 80 - protein, 535 aa, Onthophagus taurus
MFKQPLTQTEIEEEIRRIMCGEDSGSDWEEDNLEPEPEDSDDESDDDNIVIAPETPVCIPQRNLFQDPPTAAREQTPTEGEESETNILEVCSSLHQYLIKPNKTSLTGKNGHKWSATVPAKCKRTAARNIIHFIPGPKGNTNNYSMFDEYFLHFFSHDILEIILQHTNAEIARRAQKYQSHANISEITKNELKALFAILIMSAANKDNHLSMKHMFNTNISGKFYRACMSGERFSFLLNCLRFDNKETRQERVLKDPLAYIREIWDIFITTCRTSYTPSSYLTIDEQLVGFRGRCPFKIYIPNKPSKYGIKIVMLCDSSSKYMIDADPYLGKLTKRNGLPLASYYVKELTKSVHGSNRNVTMDNWFTSVPLADELLKDPYKLTILGTLRKNKKEIPTELLNVKGRNPNTSMFCYVRQKTLLSYMPKRQKIVLLLSTLHEGAEITATGKPAIIQNYNQTKGGVDTFDQMCSNMNSCRWLSFIFYLLGYDKYWKHKFIRHLFYKHTKYEQKTLIQVPIYVRIKSSSCRTVDEGQVEY

SEQ ID NO: 81 - DNA, 338 bp, Onthophagus taurus
  source 1..338: mol_type = genomic DNA
ttaaccctagaatactggacattcgtaaattttacgacgctcatcaaacttcgttaagatgcggatacttccggttcacacaccgtcgcgcgcttctaggtaccttttcatttagttgtgaactggtcagtgctggctgcagttgctgtatagtttgtagctaaatagggtattttgtaatcgcgtcgtaaattttacgaaactccagtatctacgtatagtttcttaataacgttctgcattcaaatacagggttagtgcgtacagtttgagaggtaaattactttatttgacacgttaaaatgtttcagtaaggtttgtgctttgtttttagaaaa

SEQ ID NO: 82 - DNA, 399 bp, Onthophagus taurus
  source 1..399: mol_type = genomic DNA
tactttgcgtagggatattcgccaagatattagtgccattctcaacatttctattgaaaatgacacacaagcatgtcaagataagaaacgcaaaatttgtttttattgtcctagtaaaaagaggcgtatgactacgacgtattgtaaccaatgcaagcatgcgatatgtggggaacaccgaggagaaatatgcactatgtgcgcaaagtaataatatatatatactatattgcttcctattttttatgttcttaaaagtacattatatgtaatactatttttgtcaactttctaaaatatatgtttttttttcaaatgtggagttttgtaaaaagtcgtaaaaattacgatagtccagtattaatgtatgcttactacagttcagtattctagggttaa

SEQ ID NO: 83 - protein, 590 aa, Vanessa tameamea
MSLSNHPDRILTLLEESDSEDENNDTASDVDNEDHLSERSNNSDSEEDASSIDSDSSENLDLLSLQNRLRQQVFRGKDGTLWQKEPGRSNVRTRSENIITELPGVKLIARDAKTVFECWNLFITQDMLYTIKTCTNSHIQERRALCADVSKQRFMSEVEISELKAFIGLLYLAGFYRSNRQNLKDLWQKDGTGIEIFRLTMSIQRFYFIQSCLRFDDKNTRAERQNLDNLAPIRELFKEFTEQCLSMYSPGENCTIDEMLVAFRGRCKFRQYIPSKPAKYGIKIFALVDSKTFYVKNLEIYAGKQPSGPYSVSNKPFDVVNRLVLPISKTHRNVTFDNWFTSYEVVSHLLNEHRLTTVGTVRKNKKQIPPQFLNIRGKELNSSTFGFQKDISLVSYIPKKNKIVLLMSSLHHDANIDQSTGDQRKPEIITYYNATKSGVDVADELSATYDVSRNSKRWPMTSFYAMLNISGINANIIYRANNTDTRMTRRHFLKKLGLDLIHDHLEVRKTQMNLPRLLRKRILDFVGGPAEEPPRKITGTRKRCQICPAKKDKKTNHTCHVCHIYICPDHIIPYCNNCSTSMAISDDVED

SEQ ID NO: 84 - DNA, 315 bp, Vanessa tameamea
  source 1..315: mol_type = genomic DNA
ttaacccggcgtgattgacgctgggtcacagtgacccagcgcgttttaaaacggccatattttttttatgagttgatcatttgttttaaccgtttaagtagctggagttacgtcccccctctccttagtcggccggtaagtttagtgctacgtcggctgcgcagcgagatacacgtgcgccaagtcaacgcgggtaagtttgacccagcgtcatcgcgggcgtcgtatttttgtggcattccttttttaattaattttatgttttattcaatgtcggtggttaatttacttattttttacagtattttgccgaca

SEQ ID NO: 85 - DNA, 362 bp, Vanessa tameamea
  source 1..362: mol_type = genomic DNA
tttcttactatttttaatataaaagtatttttgatctcttatctataacctaagattgttagttcctattattcgtaagaaaagtctattaaaattgttaaaagtagaacaaactaatgaatgttaaattttgaaccttgattttgcatgtgttactttggaaaccaataaaagtatgattgatttaatataatggcataattttattttataacaagcgcagacgaccctttttaacaaataactaaaaaaaataagataacgtcttttatttcgcaaaaaatttactttataatgaggggtaattgtgacccagtgtcattgaccacgtaatttttttcacgtcaatcacgccgggttaa